(19) United States
(12) Patent Application Publication
(10) Pub. No.: US 2024/0306956 A1
(43) Pub. Date: Sep. 19, 2024

(54) CONTINUOUS MULTI-ANALYTE SENSOR SYSTEMS

(71) Applicant: Dexcom, Inc., San Diego, CA (US)

(72) Inventors: Stacy Hunt DUVALL, San Diego, CA (US); Renaud BONZOM, San Diego, CA (US); Jose Ferney RIVERA MIRANDA, San Diego, CA (US); Ted LEE, San Diego, CA (US); Morgan ROBINSON, San Diego, CA (US); Sanaz PILEHVAR, San Diego, CA (US); Pradeep Ramiah RAJASEKARAN, San Diego, CA (US); Wenjie LAN, San Diego, CA (US); Nicolas Robert MEDJO, San Diego, CA (US); Didier SAGAN, San Diego, CA (US); Chris W. DRING, San Diego, CA (US)

(21) Appl. No.: 18/604,461

(22) Filed: Mar. 13, 2024

Related U.S. Application Data
(60) Provisional application No. 63/490,589, filed on Mar. 16, 2023.

(51) Int. Cl. A61B 5/145; A61B 5/1473

(52) U.S. Cl. CPC A61B 5/14546; A61B 5/1473; A61B 5/14532; A61B 2560/0219; A61B 2562/12; A61B 2562/225

(57) ABSTRACT

Various embodiments disclosed relate to analyte sensor configurations. The present disclosure can include planar analyte sensors or coaxial analyte sensors. The planar analyte sensors can include one or more insulating and conductive layers and a substrate layered on each other. The coaxial analyte sensors can include one or more co-extruded wire electrodes with a substrate. The continuous analyte monitoring systems discussed herein can be configured to monitor one or more analytes to provide predictive and real-time health data and benefits.

### CROSS-REFERENCE TO RELATED APPLICATIONS

This application claims priority to U.S. Application Ser. No. 63/490,589, filed Mar. 16, 2023, entitled “CONTINUOUS MULTI-ANALYTE SENSOR SYSTEMS,” which is hereby incorporated by reference in its entirety.

### TECHNICAL FIELD

The present development relates generally to medical devices such as analyte sensors, and more particularly, but not by way of limitation, to architectures and manufacturing techniques for analyte sensors.

### BACKGROUND

Various systems may be used to monitor one or more analyte levels in a subject or subjects. These levels may be monitored and analyzed to determine various aspects of a subject's current health, as well as to predict future states of health, including the development and monitoring of conditions such as metabolic conditions. Accordingly, there is a need for improved analyte monitoring systems that can safely, reliably, and effectively provide an accurate monitoring of analyte levels in a subject.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY OF THE DISCLOSURE

The present disclosure provides a variety of architectures and methods of manufacturing and use of analyte sensors, such as continuous glucose sensors useful for diabetes patients. The analyte sensors can, for example, be used for monitoring blood glucose levels in diabetes patients. Discussed herein, such analyte sensor architectures can include planar and coaxial structures.

In an example, an analyte sensor can include a substrate extending between a proximal portion and a distal portion, a first electrode, a second electrode, and an insulating layer between the first electrode and the second electrode. The substrate can be planar. The first electrode can be a working electrode. The first electrode and the second electrode can each be substantially parallel to the substrate, and the first electrode and the second electrode can each be planar. The insulating layer can be planar. The first electrode can extend further towards the distal portion of the substrate than the second electrode.

In an example, an analyte sensor can include a substrate extending between a proximal portion and a distal portion, a first electrode extending along the substrate between the proximal portion and the distal portion, a second electrode extending along the substrate between the proximal portion and the distal portion, and an insulating portion electrically separating the first electrode and the second electrode. The substrate can include a cylindrical member having a central axis ringed by a plurality of concentric circles of increasing radii. The concentric circles can be visible by a cross section of the substrate. The first electrode can be a working electrode. The first and second electrodes can each be aligned with a one of the pluralities of concentric circles, and the first electrode can extend further towards the distal portion of the substrate than the second electrode.

In an example, a method of making an analyte sensor can include aligning one or more insulating layers and one or more conducting layers in an alternating fashion, laminating the one or more insulating layers and the one or more conductive layers together, and exposing at least two electrodes by selectively removing portions of the one or more insulating layers.

In an example, a method of making an analyte sensor can include co-extruding a substrate and at least two electrode wires in a cylindrical shape to produce an analyte sensor, wherein each of the at least two electrodes aligns with a different concentric circle within the cylindrical shape, singularizing the produced analyte sensor into a plurality of individual analyte sensors, and selectively removing predetermined portions of the substrate material on each of the individual analyte sensors to expose sensing areas of the at least two electrodes.

In an example, a continuous analyte sensor can include: a substrate having a first side and a second side opposite the first side, wherein the substrate is planar; a first working electrode on the substrate; a second working electrode on the substrate; a reference electrode on the substrate, wherein the first working electrode, the second working electrode, and the reference electrode are each planar electrodes, wherein at least two of the first working electrode, the second working electrode, or the reference electrode are on the first side of the substrate, and any remaining planar electrodes are on the second side of the substrate, wherein the at least two of first working electrode, the second working electrode, and the reference electrode are co-planar with each other; and an interconnect extending through the substrate between the first side and the second side, the interconnect in electrical communication with one of the reference electrode, the first working electrode, or the second working electrode.

In an example, a sensor can include: a substrate having a first side and a second side opposite the first side, wherein the substrate is planar; a first sensor system on the substrate, the first sensor system being a continuous analyte sensor configured to collect a first type of measurement, wherein the first sensor system comprises: a working electrode; a reference electrode on the substrate, wherein the working electrode and the reference electrode are each planar electrodes; and at least one analyte-sensing membrane extending over the at least one working electrode; and a second sensor system on the substrate, wherein the second sensor system is configured to collect a second type of measurement different than the first type of measurement.

In an example, a continuous analyte sensor can include: a planar substrate having a distal portion and a proximal portion connected by a junction portion, wherein the distal portion and the proximal portion are connected through the junction portion at an angle between 70 and 110 degrees; a first electrode on the distal portion of the substrate and a first connection pad on the proximal portion, the first electrode and the first connection pad electrically coupled through a first trace routed through the junction portion; a second electrode on the distal portion of the substrate and a second connection pad on the proximal portion, the second electrode and the second connection pad electrically coupled through a second trace routed through the junction portion; and at least one analyte-sensing membrane extending over one or more of the first electrode and the second electrode.

In an example, a method of making a planar analyte sensor can include layering a first insulating material, a first conductive material, and a second conductive material on a first side of a substrate; exposing a portion of first conductive material to form a first electrode by selectively removing portions of the first insulating material; exposing a portion of the second conductive material to form a second electrode by selectively removing portions of the first insulating material; and layering a second insulating material and a third conductive material on a second side of the substrate opposite the first side; exposing a portion of the third conductive material to form a third electrode by selectively removing portions of the third insulating material; and depositing an analyte-sensing membrane onto two of the first electrode, the second electrode, or the third electrode.

In an example, a method of making a plurality of analyte sensors can include: producing a plurality of sensor substrates from a substrate material sheet, wherein each of the plurality of sensor substrates is aligned on the substrate material sheet; forming a working electrode and a reference electrode on each of the plurality of sensor substrates; applying an analyte-sensitive membrane on each of the working electrodes on each of the plurality of sensor substrates.

In an example, a method of making an analyte sensor can include: aligning a plurality of insulating layers and a plurality of conducting layers in an alternating fashion; laminating the plurality of insulating layers and the plurality of conductive layers together; and exposing at least two electrodes by selectively removing portions of the plurality of insulating layers.

In an example, an analyte sensor can include: a sensor substrate; a first electrode mechanically coupled to the sensor substrate; a first electrode trace mechanically coupled to the sensor substrate and electrically coupled to the first electrode; a second electrode mechanically coupled to the sensor substrate; a second electrode trace mechanically coupled to the sensor substrate and electrically coupled to the first electrode; and an analog front end (AFE) circuit mechanically coupled to the sensor substrate, electrically coupled to the first electrode trace, and electrically coupled to the second electrode trace.

In an example, an analyte sensor system can include: an analyte sensor, the analyte sensor comprising: a sensor substrate; a first electrode mechanically coupled to the sensor substrate; a first electrode trace mechanically coupled to the sensor substrate and electrically coupled to the first electrode; a second electrode mechanically coupled to the sensor substrate; a second electrode trace mechanically coupled to the sensor substrate and electrically coupled to the first electrode; and an analog front end (AFE) circuit mechanically coupled to the sensor substrate, electrically coupled to the first electrode trace, and electrically coupled to the second electrode trace; and a sensor electronics assembly; and a connector electrically coupling the analog front end circuit to the sensor electronics assembly.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a diagram showing one example of an environment including an analyte sensor system.

FIG. 2 is a diagram showing one example of a medical device system including the analyte sensor system of FIG. 1.

FIG. 3A is an illustration of an example analyte sensor.

FIG. 3B is a cross-sectional view through the sensor of FIG. 3A on line B-B.

FIG. 3C is a cross-sectional view through the sensor of FIG. 3A on line C-C.

FIG. 3D is a cross-sectional view through the sensor of FIG. 3A on line D-D.

FIGS. 4A-4G illustrate a single sided, co-planar analyte sensor assembly, in accordance with an example.

FIGS. 5A-5K illustrate a single sided, stacked analyte sensor assembly, in accordance with an example.

FIGS. 6A-6E illustrate a double-sided, co-planar un-connected analyte sensor, in accordance with an example.

FIGS. 7A-7I illustrate a double-sided, co-planar connected analyte sensor, in accordance with an example.

FIGS. 8A-8F illustrate a double-sided, co-planar connected analyte sensor, in accordance with an example.

FIG. 9 illustrates a method of making a co-planar analyte sensor assembly, in accordance with an example.

FIGS. 10A-10B illustrate a method of making a multi-layer, co-planar analyte sensor assembly, in accordance with an example.

FIGS. 11A-11C illustrate a method of making a co-planar analyte sensor assembly, in accordance with an example.

FIG. 12 illustrates a method of making a co-planar analyte sensor assembly, in accordance with an example.

FIG. 13 illustrates a method of making a co-planar analyte sensor assembly, in accordance with an example.

FIGS. 14A-14D illustrate a method of skiving co-planar analyte sensor assemblies, in accordance with an example.

FIGS. 15A-15D illustrate a coaxial analyte sensor assembly having radial wires in an insulating polymer, in accordance with an example.

FIGS. 16A-16D illustrate a coaxial analyte sensor assembly having radial wires in an insulating polymer, in accordance with an example.

FIGS. 17A-17D illustrate a coaxial analyte sensor assembly having radial wires in an insulating polymer, in accordance with an example.

FIG. 18 illustrates a coaxial analyte sensor assembly including wires embedded in a photoresist insulating polymer, in accordance with an example.

FIGS. 19A-19B illustrate a coaxial analyte sensor assembly including a polymer core, in accordance with an example.

FIGS. 20A-20B illustrate a coaxial analyte sensor assembly including a wire core, in accordance with an example.

FIGS. 21A-21B illustrate a double-sided, stacked planar analyte sensor, in accordance with an example.

FIGS. 22A-22B illustrate a double-sided, co-planar analyte sensor, in accordance with an example.

FIGS. 23A-23D illustrate a double-sided, co-planar analyte sensor, in accordance with an example.

FIGS. 24A-24B illustrate a double-sided, co-planar analyte sensor, in accordance with an example.

FIGS. 25A-25E illustrate a double-sided, co-planar analyte sensor, in accordance with an example.

FIGS. 26A-26C illustrate a double-sided, co-planar analyte sensor, in accordance with an example.

FIGS. 27A-27D illustrate a double-sided, co-planar analyte sensor, in accordance with an example.

FIGS. 28A-28C illustrate a double-sided, co-planar analyte sensor, in accordance with an example.

FIGS. 29-32 illustrate sensor tip designs, in accordance with an example.

FIG. 33 illustrates a sensor tip design, in accordance with an example.

FIG. 34 illustrates a sensor tip design, in accordance with an example.

FIGS. 35A-35B illustrate an L-shaped sensor, in accordance with an example.

FIG. 36 illustrates an L-shaped sensor, in accordance with an example.

FIGS. 37A-37D illustrate a cuboidal sensor, in accordance with an example.

FIG. 38 illustrates a cuboidal sensor on a circuit board, in accordance with an example.

FIGS. 39A-39D depict a method of making a cuboidal sensor, in accordance with an example.

FIG. 40 depicts a flow diagram depicting a method of making planar analyte sensor membranes, in accordance with an example.

FIG. 41 is a diagram showing one example of an analyte sensor system including an analyte sensor electrically coupled to sensor electronics via a connector.

FIG. 42 is a diagram showing one example arrangement of the analog front end circuit of FIG. 41.

FIG. 43 is a diagram showing one example arrangement of an analyte sensor comprising an analog front end circuit.

FIG. 44 is a diagram showing another example arrangement of the analyte sensor of FIG. 43.

FIG. 45 is a diagram showing another example arrangement of an analyte sensor comprising an analog front end circuit.

FIG. 46 is a diagram showing an example of the analyte sensor of FIG. 45 comprising an enclosure that is molded onto the sensor substrate.

FIG. 47 is a diagram showing another example of the analyte sensor.

FIG. 48 is a diagram showing one example of an analyte sensor system including an analyte sensor electrically coupled to sensor electronics via a contactless connector.

FIG. 49 is a diagram showing one arrangement of an analyte sensor system comprising a sensor substrate with a coil positioned thereon.

FIG. 50 is a diagram showing another arrangement of the analyte sensor system including sensor electronics via a contactless connector.

### DETAILED DESCRIPTION

The present disclosure describes, among other things, analyte sensors, including architectures and methods of making, both planar and cylindrical for more accurate, reproducible analyte monitoring results. The designs can include multiple sensing surfaces to allow the sensing of more than one analyte on a single sensor. In some cases, multiple coating layers can be used to ensure proper function of the analyte sensors. The analyte sensors can be amenable to high speed and high through-put processes and allow for efficient and flexible electronics manufacturing methods.

The example analyte sensors described herein can be placed in contact with bodily fluid of a host to measure a concentration of an analyte, such as glucose, in the bodily fluid. In other examples, two or more analytes as discussed herein may be monitored instead of or in addition to glucose. In some examples, the analyte sensor is inserted subcutaneously, under the skin of the host, and thereby placed in contact with interstitial fluid below the skin to measure the concentration of the analyte in the interstitial fluid.

When the analyte sensor is exposed to one or more analytes, a signal is generated and measured. In one example, the signal is generated via an electrochemical reaction between the analyte sensor and the analyte. This electrochemical reaction can cause the analyte sensor to generate a raw sensor signal indicating the analyte concentration. In some examples, the analyte sensor can include a working electrode (WE) and a reference electrode (RE). In other examples, the analyte sensor can also include a counter electrode (CE). In the presence of one or more analytes, the electrochemical reaction can cause a current to flow between the working electrode and the counter electrode, where the raw sensor signal can be based on the current. If present, the reference electrode can provide a stable reference potential and conducts very little current. In some cases, potentiometric measurements can be used in addition to such measurements obtained via amperometry.

In example two-electrode configurations, the counter electrode can be omitted. In this case, the electrochemical reaction between the analyte sensor and the analyte can cause a current formed between the working electrode and the reference electrode. Accordingly, the reference electrode can both conduct a current, like the counter electrode in a three-electrode configuration and provide a stable reference potential. The reference electrode in a two-electrode configuration can sometimes be referred to as a counter-reference electrode. Herein, the term reference electrode can be used to refer to the reference electrode of a three-electrode configuration, the counter-reference electrode of a two-electrode configuration, or similar electrodes in other configurations, depending upon the example electrode configuration.

In use, sensor electronics can apply a bias potential between the working electrode and the reference (e.g., counter-reference) electrode. In a two-electrode configuration, the applied bias can promote the electrochemical reaction between the analyte and the analyte sensor, resulting in a current between a working electrode and a reference (e.g., counter-reference) electrode. The current can make up all or part of the raw sensor signal. The bias potential may be positive or negative, and may be toggled between positive and negative values, or otherwise among and between values, depending upon the analyte or analytes being monitored.

Many analyte sensors are currently manufactured for detection of a single analyte, thus potentially requiring multiple sensors or even multiple systems to detect and monitor multiple analytes simultaneously. In some examples, the architecture of the sensors is associated with the types or number of analytes that can be monitored. Accordingly, in some examples as discussed herein, the sensor architecture can be configured to detect and monitor two or more analytes simultaneously, or in an overlapping or toggling fashion. Improved functionality and extended use of continuous monitoring sensors to detect multiple analytes on a single sensor could thus be useful. Similarly, it may be desirable in some examples to have a sensor architecture that can be rapidly and cost-effectively produced, for example, to be able to meet the needs of the healthcare community, including underserved communities.

Discussed herein, two different classes of analyte sensor architectures are discussed: planar configurations and coaxial configurations. Each of these architectures can potentially provide multiple benefits compared to previous wire-based architectures.

For example, planar based analyte sensors can be scaled up for production, while still providing flexible geometries. Particularly, planar configurations can be amenable to high speed, high throughout processes, such as sheet-to-sheet or reel-to-reel manufacturing methods. Such flexible electronic manufacturing methods can include photolithography, plating, or high-definition printing methods, such as inkjet printing or screen printing. Planar architectures can additionally allow for multi-analyte sensing.

Coaxial based analyte sensors can incorporate multiple sensing surfaces, allowing for efficient sensing of more than one analyte on a single sensor. Production of coaxial or cylindrical sensors can also be easily scaled, as production can be accomplished by extrusion and coating, such as discrete dispense, spray coating, or dip coating. These methods can be cost and time effective, enabling the rapid production of safe, reliable, and effective continuous analyte and continuous multi-analyte monitoring devices. Continuous analyte monitoring systems which employ the sensor architectures discussed herein may enable increased wear time of sensors, shortened break-in time improved accuracy, decreased drift, as well as increased patient comfort and potentially increased compliance with pharmaceutical regimens or other health or nutrition plans including corrective or preventive measures.

### Definitions

The terms and phrases “analyte measuring device,” “biosensor,” “sensor,” “sensing region,” and “sensing mechanism” as used herein are broad terms and phrases, and are to be given their ordinary and customary meaning to a person of ordinary skill in the art (and are not to be limited to a special or customized meaning), and refer without limitation to the area of an analyte-monitoring device responsible for the detection of, or transduction of a signal associated with, a particular analyte or combination of analytes. In one example, such devices are capable of providing specific quantitative, semi-quantitative, qualitative, semi qualitative analytical information using a biological recognition element combined with a transducing (detecting) element.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 weight (wt) % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The terms “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, sweat, cerebral spinal fluid, lymph fluid or urine) that can be analyzed. Analytes can include naturally occurring substances, artificial substances, metabolites, and/or reaction products. In some examples, the analyte for measurement by the sensing regions, devices, and methods is glucose. However, other analytes are contemplated as well, including but not limited to a carboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatinine, creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free ß-human chorionic gonadotropin; free erythrocyte porphyrin; frec thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol, glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; phenobarbitone; phenytoin; phytanic/pristanic acid; potassium (or other blood electrolytes), progesterone; prolactin; prolidase; purine nucleoside phosphorylase; quinine; reverse tri-iodothyronine (rT3); selenium; serum pancreatic lipase; sissomicin; sodium, somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalis, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniac, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Treponema pallidum, Trypanosoma cruzi/rangeli, vesicular stomatitis virus, Wuchereria bancrofti, yellow fever virus); specific antigens (hepatitis B virus, HIV-1); succinyl acetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and histamine.

The term “bioactive agent” as used herein is a broad term, and is to be given its ordinary and customary meaning to a person of ordinary skill in the art (and is not to be limited to a special or customized meaning), and refers without limitation to any substance that has an effect on or elicits a response from living tissue.

The phrases “biointerface membrane” and “biointerface layer” and “biointerface/drug releasing membrane” and “biointerface/drug releasing layer” as used interchangeably herein is a broad phrase, and is to be given its ordinary and customary meaning to a person of ordinary skill in the art (and is not to be limited to a special or customized meaning), and refers without limitation to a permeable membrane or layer that functions as an interface between host tissue and an implantable device.

The phrase “barrier cell layer” as used herein is a broad term, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The terms “bioresorbable” or “bioabsorbable” as used herein are broad terms, and are to be given their ordinary and customary meaning to a person of ordinary skill in the art (and are not to be limited to a special or customized meaning), and refer without limitation to materials that can be absorbed, or lose substance, in a biological system.

The phrase “cell processes” as used herein is a broad term, and is to be given its ordinary and customary meaning to a person of ordinary skill in the art (and is not to be limited to a special or customized meaning), and refers without limitation to pseudopodia of a cell.

The phrase “cellular attachment” as used herein is a broad term, and is to be given its ordinary and customary meaning to a person of ordinary skill in the art (and is not to be limited to a special or customized meaning), and refers without limitation to adhesion of cells and/or cell processes to a material at the molecular level, and/or attachment of cells and/or cell processes to microporous material surfaces or macroporous material surfaces. One example of a material used in the prior art that encourages cellular attachment to its porous surfaces is the BIOPORETM cell culture support marketed by Millipore (Bedford, Mass.), and as described in Brauker et al., U.S. Pat. No. 5,741,330.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The term “co-continuous” as used herein is a broad term, and is to be given its ordinary and customary meaning to a person of ordinary skill in the art (and is not to be limited to a special or customized meaning), and refers without limitation to a solid portion or cavity wherein an unbroken curved line in three dimensions can be drawn between two sides of a membrane. The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

Further examples of continuous analyte sensors can be found in, for example, U.S. Pat. Nos. 8,828,201, Simpson, et al.; 9,131,885 Simpson, et al.; 9,237,864, Simpson, et al.; and 9,763, 608, Simpson, et al., each of which is incorporated by reference in its entirety herein.

The phrase “continuous analyte sensing” as used herein is a broad term, and is to be given its ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every 5 seconds or less to about 10 minutes or more, preferably from about 10, 15, 20, 25, 30, 35, 40, 45, 50, 55, or 60 second to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes.

The term “co-planar” or “coplanar” as used herein may refer two or more electrodes or other components on a substrate, where those two or more electrodes or other components reside substantially in the same plane.

The term “coupled” as used herein may refer to two or more system elements or components that are configured to be at least one of electrically, mechanically, thermally, operably, chemically or otherwise attached.

The term “removably coupled” as used herein may refer to two or more system elements or components that are configured to be or have been electrically, mechanically, thermally, operably, chemically, or otherwise attached and detached without damaging any of the coupled elements or components.

The term “permanently coupled” as used herein may refer to two or more system elements or components that are configured to be or have been electrically, mechanically, thermally, operably, chemically, or otherwise attached but cannot be uncoupled without damaging at least one of the coupled elements or components.

The phrase “defined edges” as used herein is a broad phrase, and is to be given its ordinary and customary meaning to a person of ordinary skill in the art (and is not to be limited to a special or customized meaning), and refers without limitation to abrupt, distinct edges or borders among layers, domains, coatings, or portions. “Defined edges” are in contrast to a gradual transition between layers, domains, coatings, or portions.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane. The domains discussed herein can be formed as a single layer, as two or more layers, as pairs of bi-layers, or as combinations thereof. The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations. For example, such as host postprandial glucose concentrations. While not wishing to be bound by theory, it is believed that drift may be the result of a local decrease in glucose transport to the sensor, due to cellular invasion, which surrounds the sensor and forms an FBC, for example. It is also believed that an insufficient amount of interstitial fluid is surrounding the sensor, which results in reduced oxygen and/or glucose transport to the sensor, for example. An increase in local interstitial fluid may slow or reduce drift and thus improve sensor performance. Drift may also be the result of sensor electronics, or algorithmic models used to compensate for noise or other anomalies that can occur with electrical signals in the picoamp range.

The phrases “drug releasing membrane” and “drug releasing layer” as used interchangeably herein are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In one example, the “drug releasing membrane” and “drug releasing layer” can be comprised of two or more domains and is typically of a few microns thickness or more. In one example the drug releasing layer and/or drug releasing membrane are substantially the same as the biointerface layer and/or biointerface membrane. Examples of drug releasing layers and membranes may be found in pending U.S. Patent Publication Number: 2022-0296867, titled “DRUG RELEASING MEMBRANE FOR ANALYTE SENSOR,” filed Mar. 17, 2022, incorporated by reference in its entirety herein; as well as in pending U.S. application Ser. No. 17/945,585, titled “DRUG RELEASING MEMBRANE FOR ANALYTE SENSOR,” filed Mar. 17, 2022, incorporated by reference in its entirety herein.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In a working electrode, a redox species is produced by an enzyme-catalyzed reaction of an analyte and can create a measurable electronic current used to determine a concentration of the analyte. In some examples of a working electrode, an oxidizable species produced by a reaction of an analyte being detected can create a measurable electronic current. For example, in the detection of glucose, glucose oxidase produces hydrogen peroxide (H2O2) as a byproduct. The H2O2 reacts with the surface of the working electrode to produce two protons (2H+), two electrons (2e−) and one molecule of oxygen (O2), which produces the electronic current being detected. In a counter electrode, a reducible species, for example, O2 is reduced at the electrode surface so as to balance the current generated by the working electrode.

The term “host” as used herein is a broad term, and is to be given its ordinary and customary meaning to a person of ordinary skill in the art (and is not to be limited to a special or customized meaning), and refers without limitation to mammals, preferably humans.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds with an oxidation potential that overlaps with the analyte to be measured.

The term “in vivo” without limitation refers to the portion of a device (for example, a sensor) adapted for insertion into and/or existence within a living body of a host.

The term “ex vivo” refers to a portion of a device (for example, a sensor) adapted to remain and/or exist outside of a living body of a host.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains and is typically constructed of materials of a few microns thickness or more, which is permeable to oxygen and is optionally permeable to, e.g., glucose or another analyte. In one example, the membrane system comprises an immobilized glucose oxidase enzyme, which enables a reaction to occur between glucose and oxygen whereby a concentration of glucose can be measured. The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a drug releasing layer with at least one bioactive agent. For example, noise of one or more exemplary biosensors as presently disclosed can be determined and then compared qualitatively or quantitatively. By way of example, obtaining a raw signal timeseries with a fixed sampling interval (in units of pA), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL, (the unit of “noise”), using a glucose sensitivity timeseries, in units of pA/mg/dL, where the glucose sensitivity timeseries is derived using a mathematical model between the raw signal and reference blood glucose measurements (e.g., obtained from Blood Glucose Meter). Optionally, the timeseries can be aggregated as desired, e.g., by hour or day. Comparison of corresponding timeseries between different exemplary biosensors with the presently disclosed drug releasing layer and one or more bioactive agents provides for qualitative or quantitative determination of improvement of noise.

The terms “operably connected,” “operably coupled,” and “operably linked” as used herein are broad terms, and are to be given their ordinary and customary meaning to a person of ordinary skill in the art (and are not to be limited to a special or customized meaning), and refer without limitation to one or more components linked to another component(s) in a manner that facilitates transmission of signals between the components. For example, one or more electrodes can be used to detect an analyte in a sample and convert that information into a signal; the signal can then be transmitted to an electronic circuit. In this example, the electrode is “operably linked” to the electronic circuit.

The term “optional” or “optionally” means that the subsequently described event or circumstance can or cannot occur, and that the description includes instances where the event or circumstance occurs and instances where it does not.

The term “planar” as used herein is to be interpreted broadly to describe sensor architecture having a substrate including a first side and a second side, and a plurality of elements arranged on one or more sides of the substrate, the elements may or may not be electrically or otherwise coupled, where the elements can include conductive or insulating layers or elements configured to operate as a circuit. A planar electrode, for example, has a generally flat or level surface.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme layer. If the sensor is deemed to be the point of reference and the enzyme layer is positioned nearer to the sensor than the biointerface layer, then the enzyme layer is more proximal to the sensor than the biointerface layer.

The phrases and terms “processor module” and “microprocessor” as used herein are broad terms, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer. The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The term “sensing membrane” as used herein is a broad term, and is to be given its ordinary and customary meaning to a person of ordinary skill in the art (and is not to be limited to a special or customized meaning), and refers without limitation to a permeable or semi-permeable membrane that can comprise one or more domains in a membrane system and that is constructed of materials having a thickness of a few microns or more, and that are permeable to reactants and/or co-reactants employed in determining the analyte of interest. As an example, a sensing membrane can comprise an immobilized glucose oxidase enzyme, which catalyzes an electrochemical reaction with glucose and oxygen to permit measurement of a concentration of glucose.

### Analyte Sensor Systems

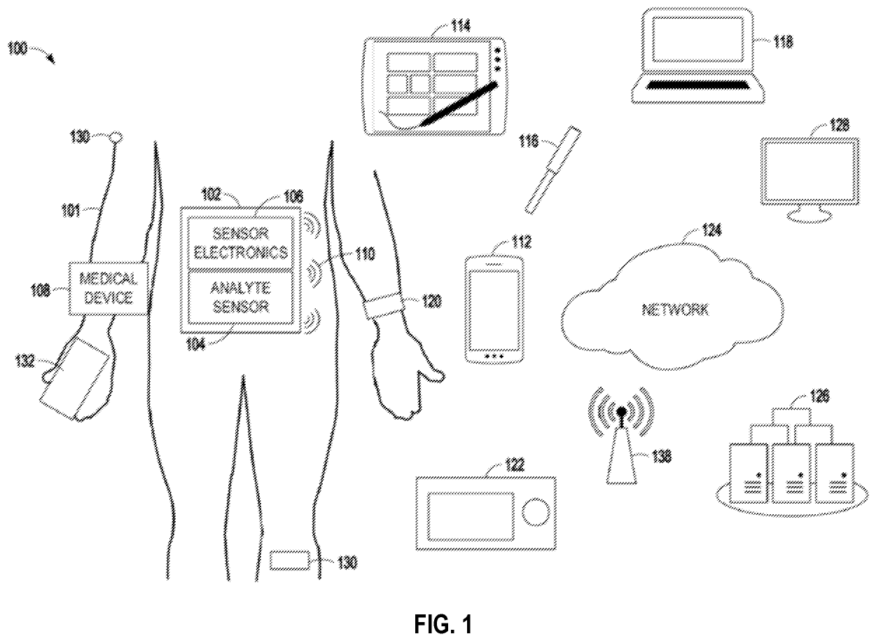

FIG. 1 is a diagram showing one example of an environment 100 including an analyte sensor system 102 configured to continuously monitor two or more analytes. The analyte sensor system 102 can be coupled to a host 101, which can be a human patient. In some cases, the host can be subject to a temporary or permanent diabetes condition or other health condition that makes analyte monitoring useful. The analyte sensor system 102 can include an analyte sensor 104 configured to detect one or more analytes. In some examples, the analyte sensor 104 can include a glucose sensor configured to measure a glucose concentration in the host 101. The analyte sensor 104 can be exposed to analyte at the host 101 in any suitable way. In some cases, the analyte sensor 104 can be fully implantable under the skin of the host 101. In other examples, the analyte sensor 104 can be wearable on the body of the host 101. Also, in some examples, the analyte sensor 104 can be a transcutaneous device (e.g., a device including a sensor such as analyte sensor 104 residing at least partially under or in the skin of a host).

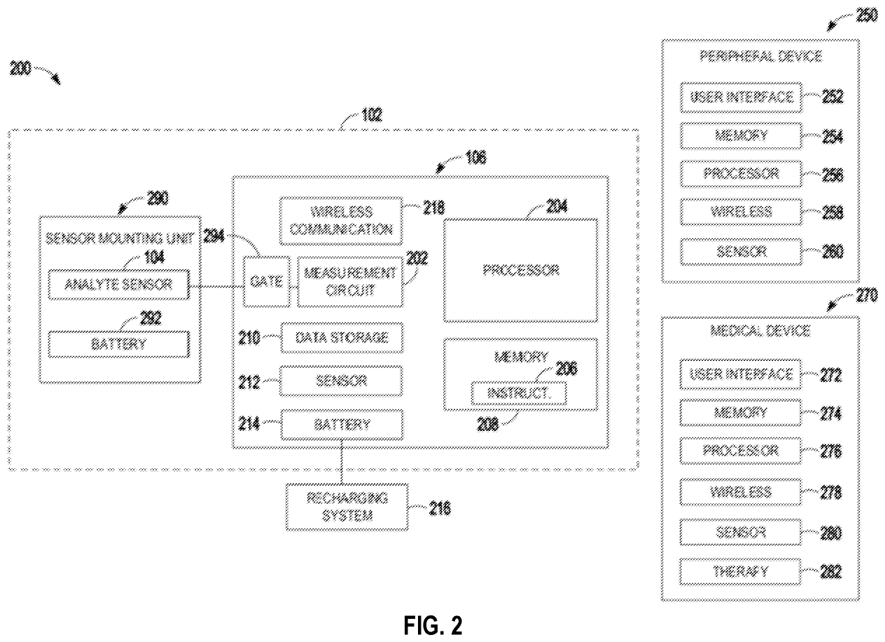

In an example, the glucose detected can be D-glucose. However, it is possible to detect any stereoisomer or blend of stereoisomers of glucose as well as any glucose in an open-chain form, cyclic form, or a mixture thereof. In the example of FIG. 1, the analyte sensor system 102 can also include sensor electronics 106. In some examples, the sensor electronics 106 and analyte sensor 104 can be provided in a single integrated package. In other examples, the analyte sensor 104 and sensor electronics 106 can be provided as separate components or modules. For example, the analyte sensor system 102 can include a disposable (e.g., single use) sensor mounting unit that can include the analyte sensor 104, a component for attaching the analyte sensor 104 to a host (e.g., an adhesive pad), and/or a mounting structure configured to receive a sensor electronics unit including some or all of the sensor electronics 106 shown in FIG. 2. The sensor electronics unit can be reusable.

The analyte sensor 104 can use a variety of methods, such as including invasive, minimally invasive, or non-invasive sensing techniques (e.g., optically excited fluorescence, microneedle, transdermal monitoring of glucose), to provide a raw sensor signal indicative of the concentration of the analyte in the host 101. The raw sensor signal can be converted into calibrated and/or filtered analyte concentration data used to provide a useful value of the analyte concentration (e.g., estimated blood glucose concentration level) to a user, such as the host or a caretaker (e.g., a parent, a relative, a guardian, a teacher, a doctor, a nurse, or any other individual that has an interest in the wellbeing of the host 101).

In some examples, the analyte sensor 104 can include a continuous glucose sensor. A continuous glucose sensor can be or include a subcutaneous, a transdermal (e.g., transcutaneous), or an intravascular device. In some cases, such a sensor or device can recurrently (e.g., periodically or intermittently) analyze sensor data. The glucose sensor can use any method of glucose measurement, including enzymatic, chemical, physical, electrochemical, spectrophotometric, polarimetric, calorimetric, iontophoretic, radiometric, immunochemical, and the like.

The environment 100 can also include a second medical device 108. The second medical device 108 can be or include a drug delivery device such as an insulin pump or an insulin pen. In some examples, the medical device 108 can include one or more sensors, such as another analyte sensor, a heart rate sensor, a respiration sensor, a motion sensor (e.g., accelerometer), posture sensor (e.g., 3-axis accelerometer), acoustic sensor (e.g., to capture ambient sound or sounds inside the body), an oxygen sensor, a temperature sensor, or others. The medical device 108 can be wearable, e.g., on a watch, glasses, contact lens, patch, wristband, ankle band, or another wearable item, or can be incorporated into a handheld device (e.g., a smartphone). In some examples, the medical device 108 can include a multi-sensor patch that can, for example, detect one or more of an analyte levels (e.g., glucose, lactate, insulin or other substance), heart rate, respiration (e.g., using impedance), activity (e.g., using an accelerometer), posture (e.g., using an accelerometer), galvanic skin response, tissue fluid levels (e.g., using impedance or pressure).

In some examples, the analyte sensor system 102 and the second medical device 108 communicate with one another. Communication between the analyte sensor system 102 and medical device 108 can occur over any suitable wired connection and/or via a wireless communication signal 110. For example, the analyte sensor system 102 can be configured to communicate using via radio frequency (e.g., Bluetooth, Medical Implant Communication System (MICS), Wi-Fi, near field communication (NFC), radio frequency identification (RFID), Zigbee, Z-Wave or other communication protocols), optically (e.g., infrared), sonically (e.g., ultrasonic), or a cellular protocol (e.g., Code Division Multiple Access (CDMA) or Global System for Mobiles (GSM)), or via a wired connection (e.g., serial, parallel, etc.).

In some examples, the environment 100 also can include a wearable sensor 130. The wearable sensor 130 can include a sensor circuit (e.g., a sensor circuit configured to detect a glucose concentration or other analyte concentration) and a communication circuit, which can, for example, be an NFC circuit. In some examples, information from the wearable sensor 130 can be retrieved from the wearable sensor 130 using a user device 132, such as a smart phone, that is configured to communicate with the wearable sensor 130 via the wearable sensor's communication circuit, for example, when the user device 132 is placed near the wearable sensor 130. For example, swiping the user device 132 over the wearable sensor 130 can retrieve sensor data from the wearable sensor 130 using NFC or other suitable wireless communication. The use of NFC communication can reduce power consumption by the wearable sensor 130, which can reduce the size of a power source (e.g., battery or capacitor) in the wearable sensor 130 or extend the usable life of the power source. In some examples, the wearable sensor 130 can be wearable on an upper arm as shown. In some examples, a wearable sensor 130 can additionally or alternatively be on the upper torso of the patient (e.g., over the heart or over a lung), which can, for example, facilitate detecting heart rate, respiration, or posture. A wearable sensor 136 can also be on the lower body (e.g., on a leg).

In some examples, an array or network of sensors can be associated with the patient. For example, one or more of the analyte sensor system 102, medical device 108, wearable device 120 such as a watch, and an additional wearable sensor 130 can communicate with one another via wired or wireless (e.g., Bluetooth, MICS, NFC or any of the other options described above,) communication. The additional wearable sensor 130 can be any of the examples described above with respect to medical device 108. The analyte sensor system 102, medical device 108, and additional wearable sensor 130 on the host 101 are provided for illustration and description and are not necessarily drawn to scale.

The environment 100 can also include one or more computing devices, such as a hand-held smart device (e.g., smart device) 112, tablet 114, smart pen 116 (e.g., insulin delivery pen with processing and communication capability), computer 118, a wearable device 120 such as a watch, or peripheral medical device 122 (which can be a proprietary device such as a proprietary user device available from DexCom), any of which can communicate with the analyte sensor system 102 via a wireless communication signal 110, and can also communicate over a network 124 with a server system (e.g., remote data center) or with a remote terminal 128 to facilitate communication with a remote user (not shown) such as a technical support staff member or a clinician.

The wearable device 120 can include an activity sensor, a heart rate monitor (e.g., light-based sensor or electrode-based sensor), a respiration sensor (e.g., acoustic- or electrode-based), a location sensor (e.g., GPS), or other sensors.

In some examples, the environment 100 can include a server system 126. The server system 126 can include one or more computing devices, such as one or more server computing devices. In some examples, the server system 126 is used to collect analyte data from the analyte sensor system 102 and/or analyte or other data from the plurality of other devices, and to perform analytics on collected data, generate or apply universal or individualized models for glucose levels, and communicate such analytics, models, or information based thereon back to one or more of the devices in the environment 100. In some examples, the server system 126 gathers inter-host and/or intra-host break-in data to generate one or more break-in characteristics, as described herein.

The environment 100 can also include a wireless access point (WAP) 138 used to communicatively couple one or more of analyte sensor system 102, network 124, server system 126, medical device 108 or any of the peripheral devices described above. For example, WAP 138 can provide Wi-Fi and/or cellular connectivity within environment 100. Other communication protocols, such as NFC or Bluetooth, can also be used among devices of the environment 100.

FIG. 2 is a diagram showing one example of a medical device system 200 including the analyte sensor system 102 of FIG. 1. In the example of FIG. 2, the analyte sensor system 102 can include sensor electronics 106 and a sensor mounting unit 290. In the example shown in FIG. 2, the sensor mounting unit 290 can include the analyte sensor 104 and a battery 292. In some examples, the sensor mounting unit 290 can be replaceable, and the sensor electronics 106 can include a debouncing circuit (e.g., gate with hysteresis or delay) to avoid, for example, recurrent execution of a power-up or power down process when a battery is repeatedly connected and disconnected or avoid processing of noise signal associated with removal or replacement of a battery. The sensor electronics 106 can be configured to continuously monitor two or more analytes. The two or more analytes can be monitored continuously or in an alternating fashion or other pattern in order to, for example, predict and prevent adverse health events such as hypoglycemia, hyperglycemia, or early stages of organ failure.

The sensor electronics 106 can include electronics components that are configured to process sensor information, such as raw sensor signals, and generate corresponding analyte concentration values. The sensor electronics 106 can, for example, include electronic circuitry associated with measuring, processing, storing, or communicating continuous analyte sensor data, including prospective algorithms associated with processing and calibration of the raw sensor signal. The sensor electronics 106 can include hardware, firmware, and/or software that enables measurement of levels of the analyte via a glucose sensor. Electronic components can be affixed to a printed circuit board (PCB), or the like, and can take a variety of forms. For example, the electronic components can take the form of an integrated circuit (IC), such as an Application-Specific Integrated Circuit (ASIC), a microcontroller, and/or a processor.

In the example of FIG. 2, the sensor electronics 106 can include a measurement circuit 202 (e.g., potentiostat) coupled to the analyte sensor 104 and configured to recurrently obtain analyte sensor readings using the analyte sensor 104. For example, the measurement circuit 202 can continuously or recurrently measure a raw sensor signal indicating a current flow at the analyte sensor 104 between a working electrode and a counter or reference (e.g., counter-reference) electrode. The sensor electronics 106 can include a gate circuit 294, which can be used to gate the connection between the measurement circuit 202 and the analyte sensor 104. For example, the analyte sensor 104 can accumulate charge over an accumulation period. After the accumulation period, the gate circuit 294 is opened so that the measurement circuit 202 can measure the accumulated charge. Gating the analyte sensor 104 can improve the performance of the analyte sensor system 102 by creating a larger signal to noise or interference ratio (e.g., because charge accumulates from an analyte reaction, but sources of interference, such as the presence of acetaminophen near a glucose sensor, do not accumulate, or accumulate less than the charge from the analyte reaction).

The sensor electronics 106 can also include a processor 204. The processor 204 is configured to retrieve instructions 206 from memory 208 and execute the instructions 206 to control various operations in the analyte sensor system 102. For example, the processor 204 can be programmed to control application of bias potentials to the analyte sensor 104 via a potentiostat at the measurement circuit 202, interpret raw sensor signals from the analyte sensor 104, and/or compensate for environmental factors. The processor 204 can also save information in data storage memory 210 or retrieve information from data storage memory 210. In various examples, data storage memory 210 can be integrated with memory 208, or can be a separate memory circuit, such as a non-volatile memory circuit (e.g., flash RAM).

The sensor electronics 106 can also include a sensor 212, which can be coupled to the processor 204. The sensor 212 can be a temperature sensor, accelerometer, or another suitable sensor. The sensor electronics 106 can also include a power source such as a capacitor or battery 214, which can be integrated into the sensor electronics 106, or can be removable, or part of a separate electronics unit. The battery 214 (or other power storage component, e.g., capacitor) can optionally be rechargeable via a wired or wireless (e.g., inductive or ultrasound) recharging system 216. The recharging system 216 can harvest energy, can receive energy from an external source or on-board source, or can harvest energy from body heat (e.g., thermoelectric effect). In various examples, the recharge circuit can include a triboelectric charging circuit, a piezoelectric charging circuit, an RF charging circuit, a light charging circuit, an ultrasonic charging circuit, a heat charging circuit, a heat harvesting circuit, or a circuit that harvests energy from the communication circuit. In some examples, the recharging circuit can recharge the rechargeable battery using power supplied from a replaceable battery (e.g., a battery supplied with a base component).

The sensor electronics 106 can also include one or more supercapacitors in the sensor electronics unit (as shown), or in the sensor mounting unit 290. For example, the supercapacitor can allow energy to be drawn from the battery 214 in a highly consistent manner to extend the life of the battery 214. The battery 214 can recharge the supercapacitor after the supercapacitor delivers energy to the communication circuit or to the processor 204, so that the supercapacitor is prepared for delivery of energy during a subsequent high-load period. In some examples, the supercapacitor can be configured in parallel with the battery 214. A device can be configured to preferentially draw energy from the supercapacitor, as opposed to the battery 214. In some examples, a supercapacitor can be configured to receive energy from a rechargeable battery for short-term storage and transfer energy to the rechargeable battery for long-term storage.

The supercapacitor can extend an operational life of the battery 214 by reducing the strain on the battery 214 during the high-load period. In some examples, a supercapacitor removes at least 10% of the strain off the battery during high-load events. In some examples, a supercapacitor removes at least 20% of the strain off the battery during high-load events. In some examples, a supercapacitor removes at least 30% of the strain off the battery during high-load events. In some examples, a supercapacitor removes at least 50% of the strain off the battery during high-load events.

The sensor electronics 106 can also include a wireless communication circuit 218, which can for example include a wireless transceiver operatively coupled to an antenna. The wireless communication circuit 218 can be operatively coupled to the processor 204 and can be configured to wirelessly communicate with one or more peripheral devices or other medical devices, such as an insulin pump or smart insulin pen.

In the example of FIG. 2, the medical device system 200 also can include an optional peripheral device 250. The peripheral device 250 can be any suitable user computing device such as, for example, a wearable device (e.g., activity monitor), such as a wearable device 120. In other examples, the peripheral device 250 can be a hand-held smart device (e.g., smartphone or other device such as a proprietary handheld device available from Dexcom), a tablet 114, a smart pen 116, or special-purpose computer 118 shown in FIG. 1. In some cases, the medical device system 200 can be incorporated into an internet of things, such as including wearable smart technology. Such wearable smart technology can include, for example, watches, belts, necklaces, earrings, bracelets, headphones, earbuds, or other wearable items.

The peripheral device 250 can include a UI 252, a memory circuit 254, a processor 256, a wireless communication circuit 258, a sensor 260, or any combination thereof. The peripheral device 250 can not necessarily include all the components shown in FIG. 2. The peripheral device 250 can also include a power source, such as a battery.

The UI 252 can, for example, be provided using any suitable input/output device or devices of the peripheral device 250 such as, for example, a touch-screen interface, a microphone (e.g., to receive voice commands), or a speaker, a vibration circuit, or any combination thereof. The UI 252 can receive information from the host or another user (e.g., instructions, glucose values). The UI 252 can also deliver information to the host or other user, for example, by displaying UI elements at the UI 252. For example, UI elements can indicate glucose or other analyte concentration values, glucose or other analyte trends, glucose or other analyte alerts, etc. Trends can be indicated by UI elements such as arrows, graphs, charts, etc.

The processor 256 can be configured to present information to a user, or receive input from a user, via the UI 252. The processor 256 can also be configured to store and retrieve information, such as communication information (e.g., pairing information or data center access information), user information, sensor data or trends, or other information in the memory circuit 254. The wireless communication circuit 258 can include a transceiver and antenna configured to communicate via a wireless protocol, such as any of the wireless protocols described herein. The sensor 260 can, for example, include an accelerometer, a temperature sensor, a location sensor, biometric sensor, or blood glucose sensor, blood pressure sensor, heart rate sensor, respiration sensor, or another physiologic sensor.

The peripheral device 250 can be configured to receive and display sensor information that can be transmitted by sensor electronics 106 (e.g., in a customized data package that is transmitted to the display devices based on their respective preferences). Sensor information (e.g., blood glucose concentration level) or an alert or notification (e.g., “high glucose level”, “low glucose level” or “fall rate alert” can be communicated via the UI 252 (e.g., via visual display, sound, or vibration). In some examples, the peripheral device 250 can be configured to display or otherwise communicate the sensor information as it is communicated from the sensor electronics 106 (e.g., in a data package that is transmitted to respective display devices). For example, the peripheral device 250 can transmit data that has been processed (e.g., an estimated analyte concentration level that can be determined by processing raw sensor data), so that a device that receives the data cannot be required to further process the data to determine usable information (such as the estimated analyte concentration level). In other examples, the peripheral device 250 can process or interpret the received information (e.g., to declare an alert based on glucose values or a glucose trend). In various examples, the peripheral device 250 can receive information directly from sensor electronics 106, or over a network (e.g., via a cellular or Wi-Fi network that receives information from the sensor electronics 106 or from a device that is communicatively coupled to the sensor electronics 106).

In the example of FIG. 2, the medical device system 200 can include an optional medical device 270. For example, the medical device 270 can be used in addition to or instead of the peripheral device 250. The medical device 270 can be or include any suitable type of medical or other computing device including, for example, the medical device 108, peripheral medical device 122, wearable device 120, wearable sensor 130, or wearable sensor 136 shown in FIG. 1. The medical device 270 can include a UI 272, a memory circuit 274, a processor 276, a wireless communication circuit 278, a sensor 280, a therapy circuit 282, or any combination thereof.

Similar to the UI 252, the UI 272 can be provided using any suitable input/output device or devices of the medical device 270 such as, for example, a touch-screen interface, a microphone, or a speaker, a vibration circuit, or any combination thereof. The UI 272 can receive information from the host or another user (e.g., glucose values, alert preferences, calibration coding). The UI 272 can also deliver information to the host or other user, for example, by displaying UI elements at the UI 252. For example, UI elements can indicate glucose or other analyte concentration values, glucose or other analyte trends, glucose or other analyte alerts, etc. Trends can be indicated by UI elements such as arrows, graphs, charts, etc.

The processor 276 can be configured to present information to a user, or receive input from a user, via the UI 272. The processor 276 can also be configured to store and retrieve information, such as communication information (e.g., pairing information or data center access information), user information, sensor data or trends, or other information in the memory circuit 274. The wireless communication circuit 278 can include a transceiver and antenna configured communicate via a wireless protocol, such as any of the wireless protocols described herein.

The sensor 280 can, for example, include an accelerometer, a temperature sensor, a location sensor, biometric sensor, or blood glucose sensor, blood pressure sensor, heart rate sensor, respiration sensor, or another physiologic sensor. The medical device 270 can include two or more sensors (or memories or other components), even though only one sensor 280 is shown in the example in FIG. 2. In various examples, the medical device 270 can be a smart handheld glucose sensor (e.g., blood glucose meter), drug pump (e.g., insulin pump), or other physiologic sensor device, therapy device, or combination thereof.

In examples where medical device 270 can include an insulin pump, the pump and analyte sensor system 102 can be in two-way communication (e.g., so the pump can request a change to an analyte transmission protocol, e.g., request a data point or request data on a more frequent schedule), or the pump and analyte sensor system 102 can communicate using one-way communication (e.g., the pump can receive analyte concentration level information from the analyte sensor system). In one-way communication, a glucose value can be incorporated in an advertisement message, which can be encrypted with a previously shared key. In a two-way communication, a pump can request a value, which the analyte sensor system 102 can share, or obtain and share, in response to the request from the pump, and any or all of these communications can be encrypted using one or more previously shared keys. An insulin pump can receive and track analyte (e.g., glucose) values transmitted from analyte sensor system 102 using one-way communication to the pump for one or more of a variety of reasons. For example, an insulin pump can suspend or activate insulin administration based on a glucose value being below or above a threshold value.

In some examples, the medical device system 200 can include two or more peripheral devices and/or medical devices that each receive information directly or indirectly from the analyte sensor system 102. Because different display devices provide many different user interfaces, the content of the data packages (e.g., amount, format, and/or type of data to be displayed, alarms, and the like) can be customized (e.g., programmed differently by the manufacturer and/or by an end user) for each particular device. For example, referring now to the example of FIG. 1, a plurality of different peripheral devices can be in direct wireless communication with sensor electronics 106 (e.g., such as an on-skin sensor electronics 106 that are physically connected to the continuous analyte sensor 104) during a sensor session to enable a plurality of different types and/or levels of display and/or functionality associated with the displayable sensor information, or, to save battery power in the analyte sensor system 102, one or more specified devices can communicate with the analyte sensor system 102 and relay (i.e., share) information to other devices directly or through a server system 126 (e.g., a network-connected data center).

### Analyte Sensor Membranes

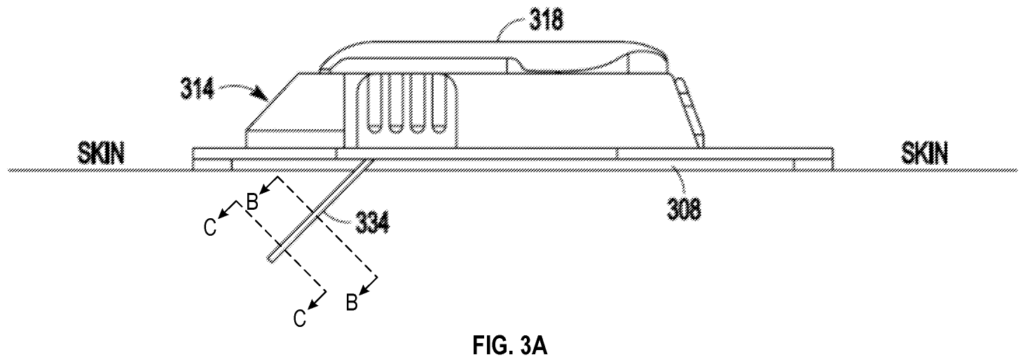

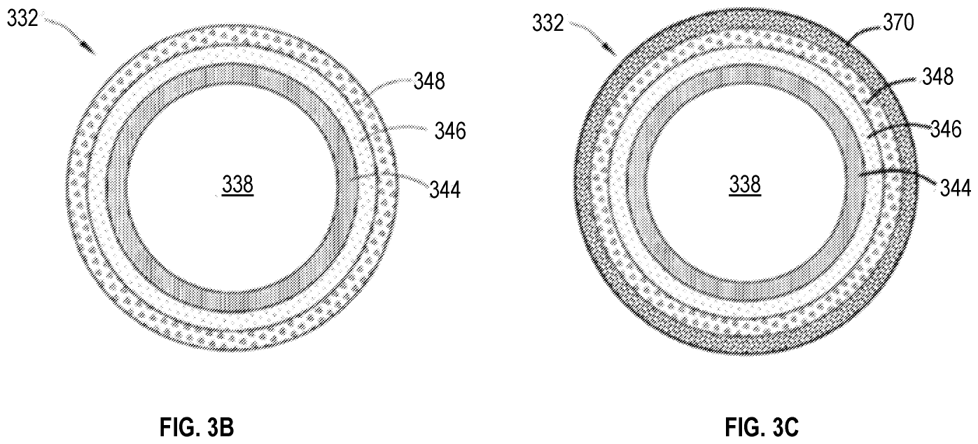

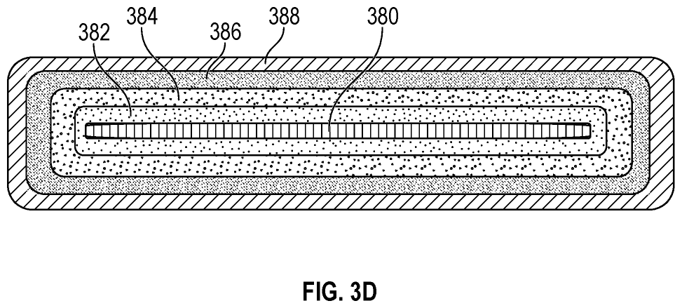

FIG. 3A is a side view of an example analyte sensor 334 that can be implanted into a host. A mounting unit 314 can be adhered to the host's skin using an adhesive pad 308. The adhesive pad 308 can be formed from an extensible material, which can be removably attached to the skin using an adhesive. Electronics unit 318 can mechanically couple to the mounting unit 314. In some examples, the electronics unit 318 and mounting unit 314 are arranged in a manner similar to the sensor electronics 106 and sensor mounting unit 290 shown in FIGS. 1 and 2. In some cases, the analyte sensor 334 does not include a separate mounting unit. In some cases, the analyte sensor 334 can be an integrated package. The analyte sensor 334 may be a coaxial sensor configuration. In other examples, the analyte sensor 334 may be a planar configuration.

FIG. 3B is a cross-sectional view through the sensor of FIG. 3A on line B-B when the sensor is configured as a coaxial configuration. FIG. 3B shows an exposed electroactive surface of at least a working electrode 338 surrounded by a sensing membrane. In general, the sensing membranes of the present disclosure include a plurality of domains or layers, for example, an interference domain 344, an enzyme domain 346, and a resistance domain 348, and can include additional domains, such as an electrode domain, a cell impermeable domain (not shown), an oxygen domain (not shown), a drug releasing membrane 370, and/or a bio interface membrane (not shown), such as described in more detail below and/or in the co-pending U.S. patent applications cited herein. However, it is understood that a sensing membrane modified for other sensors, for example, by including fewer or additional domains is within the scope of the present disclosure.

Shown in FIG. 3C is a cross-sectional view of a planar version of a sensor. FIG. 3C shows an exposed electroactive surface of at least a working electrode 380 surrounded by a sensing membrane. Similar to the circular sensor shown in FIG. 3B, the planar version can include a sensing membrane with multiple layers or domains. For example, the planar version can include an interference domain 382, an enzyme domain 384, and resistance domain 386, in addition to other variations of domains, such as drug releasing membrane 388 as discussed above.

In some examples, one or more domains of the sensing membranes are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. Co-pending U.S. patent application Ser. No. 10/838,912, which is incorporated herein by reference in its entirety, describes biointerface and sensing membrane configurations and materials that can be applied to the presently disclosed sensor.

The sensing membrane can be deposited on the electroactive surfaces of the electrode material using known thin or thick film techniques (for example, spraying, electro-depositing, dipping, or the like). It is noted that the sensing membrane that surrounds the working electrode does not have to be the same structure as the sensing membrane that surrounds a reference electrode, etc. For example, the enzyme domain deposited over the working electrode does not necessarily need to be deposited over the reference and/or counter electrodes. Depending upon the example, the sensor core, including the electrodes, and/or the membrane system may be formed in various manners, Examples of methods of forming the sensors, membrane systems, and sensor systems discussed herein may be found in currently pending U.S. patent application Ser. No. 16/452,364. Boock et al., incorporated by reference in its entirety herein.

In the illustrated example, the sensor is an enzyme-based electrochemical sensor, wherein the working electrode 338 measures a product (including but not limited to hydrogen peroxide) produced by the enzyme catalyzed reaction of glucose. In some cases, non-enzyme-based sensors can be used, such as oxygen sensors. The product detected creates a measurable electronic current (for example, detection of glucose utilizing glucose oxidase produces hydrogen peroxide as a by-product, H2O2 reacts with the surface of the working electrode producing two protons (2H+), two electrons (2e−) and one molecule of oxygen (O2) which produces the electronic current being detected), such as described in more detail above and as is appreciated by one skilled in the art. Preferably, one or more potentiostat is employed to monitor the electrochemical reaction at the electroactive surface of the working electrode(s). The potentiostat applies a constant potential to the working electrode and its associated reference electrode to determine the current produced at the working electrode. The current that is produced at the working electrode 338 (and flows through the circuitry to the counter electrode) is substantially proportional to the amount of H2O2 that diffuses to the working electrode. The output signal is typically a raw data stream that is used to provide a useful value of the measured analyte concentration in a host to the host or doctor, for example.

Some alternative analyte sensors that can benefit from the systems and methods of the present disclosure include U.S. Pat. No. 5,711,861 to Ward et al., U.S. Pat. No. 6,642,015 to Vachon et al., U.S. Pat. No. 6,654,625 to Say et al., U.S. Pat. No. 6,565,509 to Say et al., U.S. Pat. No. 6,514,718 to Heller, U.S. Pat. No. 6,465,066 to Essenpreis et al., U.S. Pat. No. 6,214,185 to Offenbacher et al., U.S. Pat. No. 5,310,469 to Cunningham et al., and U.S. Pat. No. 5,683,562 to Shaffer et al., U.S. Pat. No. 6,579,690 to Bonnecaze et al., U.S. Pat. No. 6,484,046 to Say et al., U.S. Pat. No. 6,512,939 to Colvin et al., U.S. Pat. No. 6,424,847 to Mastrototaro et al., U.S. Pat. No. 6,424,847 to Mastrototaro et al., for example. All of the above patents are incorporated in their entirety herein by reference and are not inclusive of all applicable analyte sensors; in general, it should be understood that the disclosed examples are applicable to a variety of analyte sensor configurations.

FIG. 3C is a cross-sectional view through the sensor of FIG. 3A on line C-C, showing a non-exposed electroactive surface of at least a working electrode 338 surrounded by a sensing membrane including a plurality of domains or layers, for example, the interference domain 344, the enzyme domain 346, and the resistance domain 348, and can include additional domains/membranes, such as an electrode domain, a cell impermeable domain (not shown), an oxygen domain (not shown), a drug releasing membrane 370, and/or a biointerface membrane 368 (not shown), such as described in more detail below. As shown in FIG. 3C, the drug releasing membrane 370 is positioned adjacent to working electrode 338 surface and does not cover working electrode 338 or the plurality of domains or layers, for example, the interference domain 344, the enzyme domain 346, and the resistance domain 348, of the sensing membrane 332. In one example, the drug releasing membrane 370 is positioned at the distal end 337 of the analyte sensor 334. In another example, the drug releasing membrane 370 straddles the electroactive portion of the working electrode 338 and does not cover the sensing membrane 332 associated with the working electrode 338.

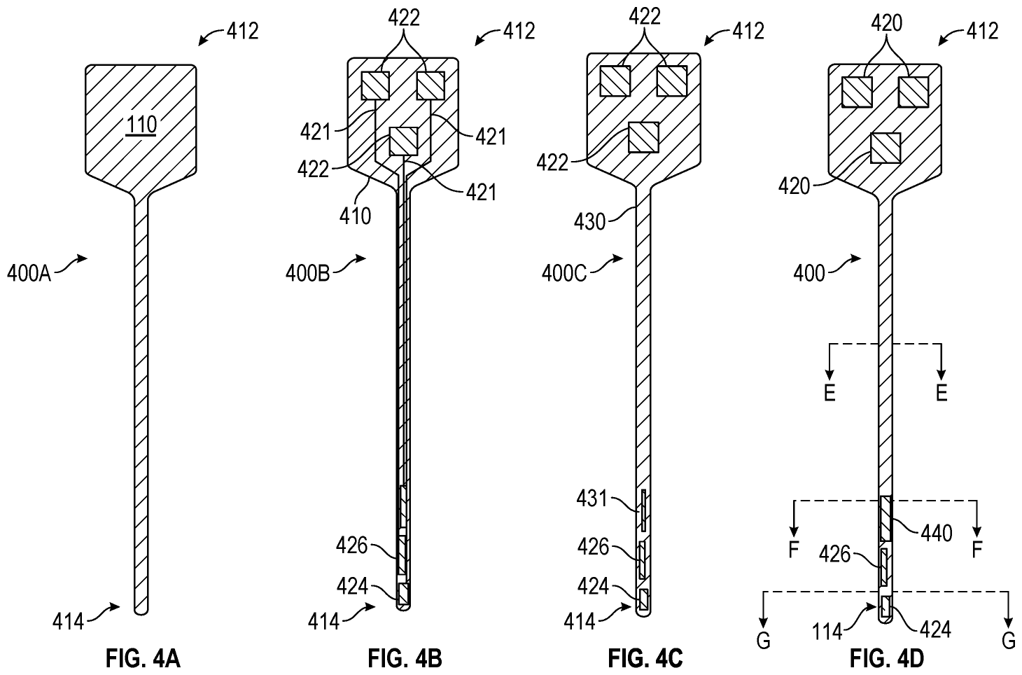

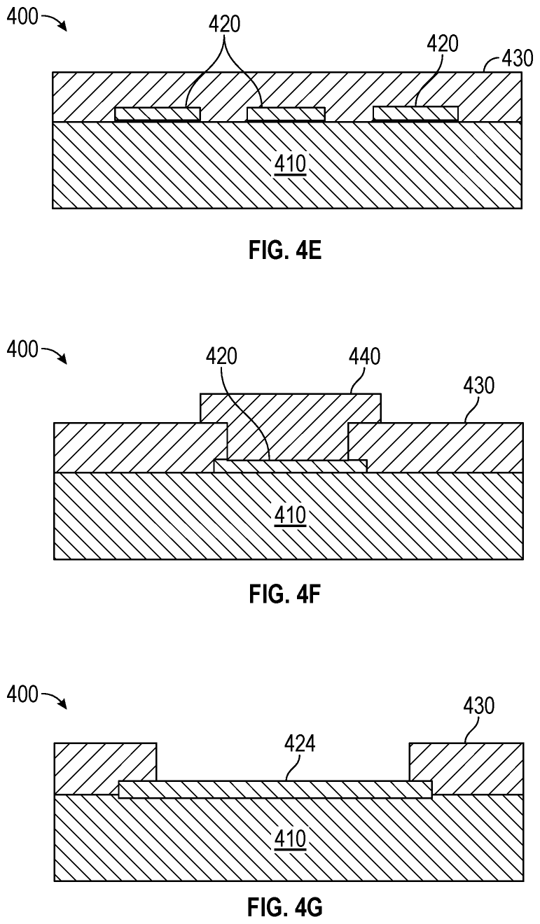

FIGS. 3A to 3C depict cross-sections of circular or coaxial sensors in examples. Similar membranes can apply to example planar analyte sensors, such as those discussed below with reference to FIGS. 4A to 8F. Sensing membranes in the example planar analyte sensors can contain the same or similar components to those membranes discussed with reference to FIGS. 3A to 3C.

### Planar Analyte Sensors

FIGS. 4A to 8F depicts schematic diagrams of planar analyte sensors. Each of the planar analyte sensors discussed herein can be configured to measure concentrations of one or more analytes. Planar analyte sensors can be readily manufactured and create reproducible results. Planar analyte sensors can be configured to monitor, including to continuously monitor, at least one analyte, and, in some examples, two or more analytes. The planar analyte sensors can be configured differently and may be described based on the geometry of their electrode layouts. The sensor types can include single-sided or double-sided layouts. In single-sided layouts, the electrodes can be conductive traces and can be in a co-planar arrangement, a stacked arrangement, or a staggered arrangement. In double-sided layouts the electrodes can be in a co-planar arrangement (aligned along a shared plane in a single layer along each substrate side), a stacked arrangement (aligned along a shared plane perpendicular to the substrate side(s)), or a staggered arrangement (offset along or more plane or axis), as well as arrangements where connector pads are on a single side of the sensor, or arrangements where connector pads are on both sides of the sensor.

FIGS. 4A-4G illustrate a single sided co-planar analyte sensor assembly 400, in accordance with an example. The sensor assembly can have a first end 412 and a second end 414. The sensor assembly 400 can include substrate 410, conductive traces 421, connector pads 422, working electrode 424, counter electrode 426, insulator 430, and reference electrode 440. In sensor assembly 400, a single-sided planar configuration is used. In the sensor assembly 400, a three-electrode sensor is shown, with a working electrode (WE) 424, a counter electrode (CE) 426 and a reference electrode (RE)440. In sensor assembly 400, the electrodes are co-planar In one or more implementations, an underlay configuration of single sided co-planar analyte sensor assembly 400 or other sensor assemblies as discussed herein may enable one or more of a reference electrode (e.g., 440) or a counter electrode (e.g., 426) to be moved off of the sensor assembly 400 or other sensor assembly as discussed herein to a position outside the body (e.g., as part of the wearable device 120 or other wearable devices as discussed herein)—rather than be deployed subcutaneously. This has the advantage of freeing up space in the host (e.g., in a wound pocket where the sensor assembly 400 is inserted), where the single sided co-planar analyte sensor assembly 400 or other sensor assemblies is deployed, for an additional working electrode and/or for detecting an additional analyte.

Moreover, by configuring the wearable device 120 or other wearable devices discussed herein to include the reference electrode 440 and counter electrode 426, such that those electrodes are externally deployed, an amount of material inserted into the body is limited. As a result, a foreign body response of a host, e.g., the response the host's immune system, may be reduced. In one or more configurations, for instance, reference electrodes (440) may include or otherwise be formed of silver chloride (AgCl). Some hosts may have sensitivity issues with silver chloride, however. Thus, configuring the wearable device 120 or other wearable devices discussed herein to include the reference electrode (440) rather than incorporating the reference electrode (440) as part of the in vivo portion of sensor assemblies discussed herein may reduce an immune response of such hosts, such as to reduce eye and/or skin irritation.

FIGS. 4A to 4D depict top-down schematic views of the sensor assembly 400 being produced. FIGS. 4E to 4G depict cross-sectional schematic views of the sensor assembly 400 at varying points along the length of the sensor assembly 400.

The sensor assembly 400 can extend between the first end 412 and the second end 414 and be substantially planar along its length, as measured from the first end 412 to the second end 414. The first end 412 can be, for example, a connection end, such as for allowing electrical connection of the sensor assembly 400 to a reader, computer, or other component for interpretation of signals detected with the sensor assembly 400. The first end 412 can host one or more connector pads 422.

The second end 414 can be, for example, a sensing end, for connection with or implantation in a patient, such as for detecting glucose or other analytes. The second end 414 can host the electrodes 424, 426, and 440. The second end 414 can be the implantable portion of the sensor assembly 400. The first end 412 of the sensor that has the connector pads 422 can be the proximal end of the sensor assembly 400. The second end 414 with the implantable portion of the sensor that contains the sensing electrodes can be the distal end of the sensor assembly 400.

Shown in FIG. 4A, the substrate 410 can extend between the first end 412 and the second end 414. The substrate 410 can be a relatively planar material, for example, the substrate 410 can be a thin flexible layer for hosting the other components. In some cases, the substrate 410 can be a polymeric film, such as liquid crystal polymer (LCP), polyimide (PI), polyethylene terephthalate (PET), combinations thereof, or similar polymeric films. The substrate 410 can have a thickness of about 25 to about 450 μm, such as a thickness of about 75 to 100 μm. In some examples, a substrate thickness of about 40 μm to about 80 μm may be used.

The conductive traces 421, connector pads 422, working electrode 424, and counter electrode 426 can be made from a conductive layer 420 built on the substrate. The connector pads 422 can be situated on or at the first end 412 of the sensor assembly 400 and allow for electrical connection of the sensor assembly 400. The working electrode 424 and the counter electrode 426 can be sensing electrodes exposed at the second end 414 of the sensor assembly 400 for implantation and sensing of an analyte in a patient environment. The conductive traces 421 can connected the electrodes 424, 426, to the connector pads 422.

Shown in FIG. 4B, the conductive layer 420 can be built up on the substrate 410 with the conductive traces 421, connector pads 422, working electrode 424, and counter electrode 426 in a single plane or layer. The conductive layer 420 can, for example, be made of a sputtered metal, such as titanium/gold/platinum or platinum/gold/platinum sputtered metal layers. In this case, relevant sensing surfaces such as at the working electrode 424 can have exposed platinum for electrical connection and sensing. The reference electrode 440 can be deposited on a base metal pad, and can be connected through additional conductive traces.

In some examples, the conductive layer 420 is formed from a single conductor, such as gold or platinum. In other examples, the conductive layer 420 or can be formed from more than one material, such as a thin palladium layer that is covered with gold and platinum. The composition, geometry, and exposed conductor surfaces can depend on the manufacturing method, desired mechanical properties, and requirements of the sensing chemistry. For example, the base conductive material can be formed by a less expensive material, such as silver, which is covered in strategic locations by platinum for the active sensing surfaces. In some cases, gold can be plated as the base conductor, which can be covered with platinum in order to provide both mechanical robustness and an active sensing surface for sensing hydrogen peroxide.

The conductive layer 420, including the working electrode 424, counter electrode 426, connector pads 422, and conductive traces 421, can be formed by a variety of techniques, such as plating, sputtering, or printing. To form the structure of patterning of the conductive layers, standard photolithographic techniques, laser ablation, or printing (e.g., inkjet or screen printing) can be used.

Although certain electrode designations are shown in the supporting document, it should be understood that the size, shape, and electrode identity can be changed depending on a specific use case, such as a particular analyte to be determined. The general size and shape of the sensor is 3 mm to 4 mm wide at the proximal end (connector end) and 300-500 μm wide in the narrow implantable distal end. The overall length of the sensor is dependent on the requirements of the wearable/inserter but are generally between 15 mm and 25 mm.

Shown in FIG. 4C, the insulator 430 can be layered on top of the conductive layer 420 as desired. Insulating materials can be referred to as “solder mask,” “dielectric,” or “insulator.” These materials can be used to protect the conductive traces from exposure to the sample matrix and environment, as well as improve the accuracy and reliability of measurements by defining the sensing electrode area. An opening 431 can be made for later deposition of the reference electrode 440.

Here, the insulator 430 can be made of an electrically insulating material deposited on top of the conductive layer to protect the conductive traces 421 and define the openings for the connector pads 422, and the electrodes 424, 426, in addition to an opening 431 for the reference electrode 440. The insulator 430 can be, for example, a thin layer of solder mask.

Shown in FIG. 4D, the reference electrode 440 material can be deposited over the designated reference electrode opening in the insulator 430. The reference electrode 440 material can be, for example, a silver/silver chloride formulation. It can be deposited on the designated sensing electrode pad. This reference electrode material can be deposited by a printing technique, such as screen printing, or by discrete dispense, such as a jet-valve dispenser.

FIGS. 4E to 4G depict cross-section of the sensor assembly 400 at varying points along the body of the assembly. FIG. 4E shows a cross-section at line E-E of FIG. 4D, in a central portion of the sensor assembly 400. At this part of the sensor assembly 400, the conductive traces 421 can be seen between the insulator 430 and the substrate 410. FIG. 4F shows a cross-section at line F-F of FIG. 4D, near the second end 414 of the sensor assembly 400. At this part of the sensor assembly 400, the reference electrode 440 can be seen on top of the conductive traces 421. FIG. 4G depicts a cross-section at line G-G of the assembly, near the second end 414. Here, the working electrode 424 can be seen. The sensor assembly 400 is a single sided, co-planar arrangement for the electrodes 424, 426, 440.

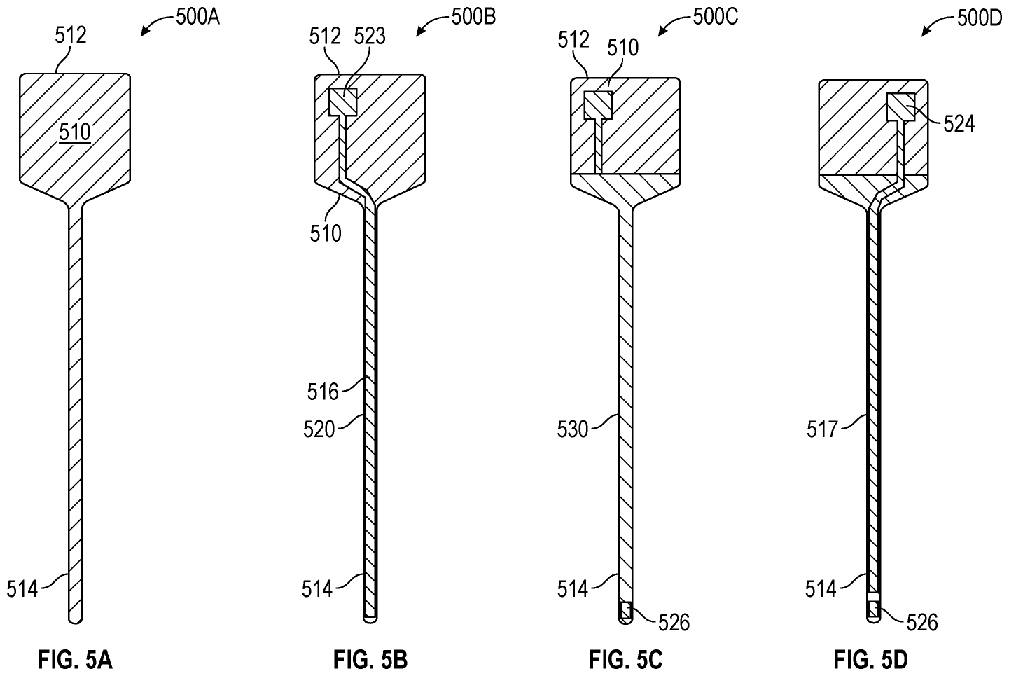

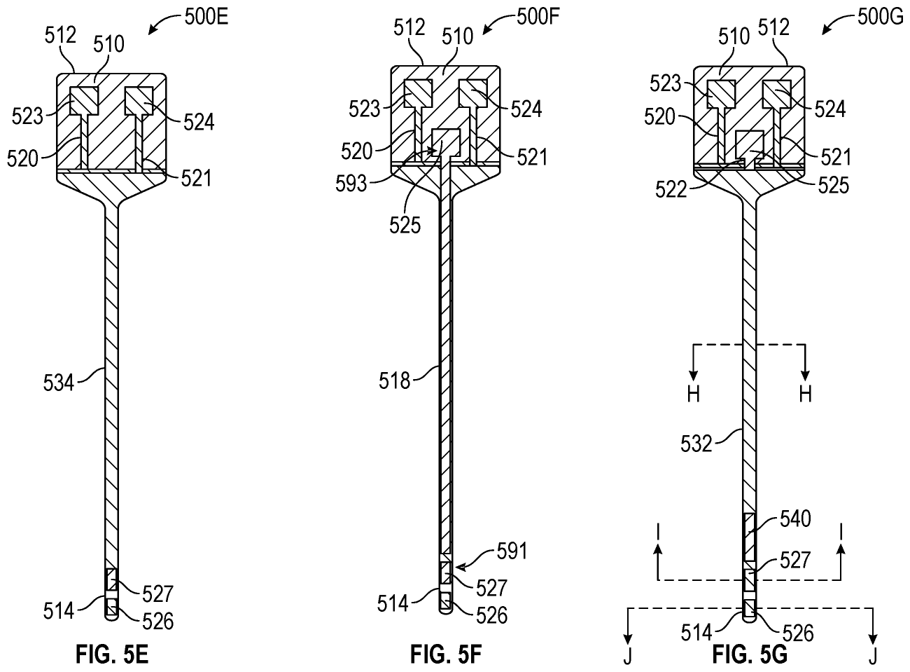

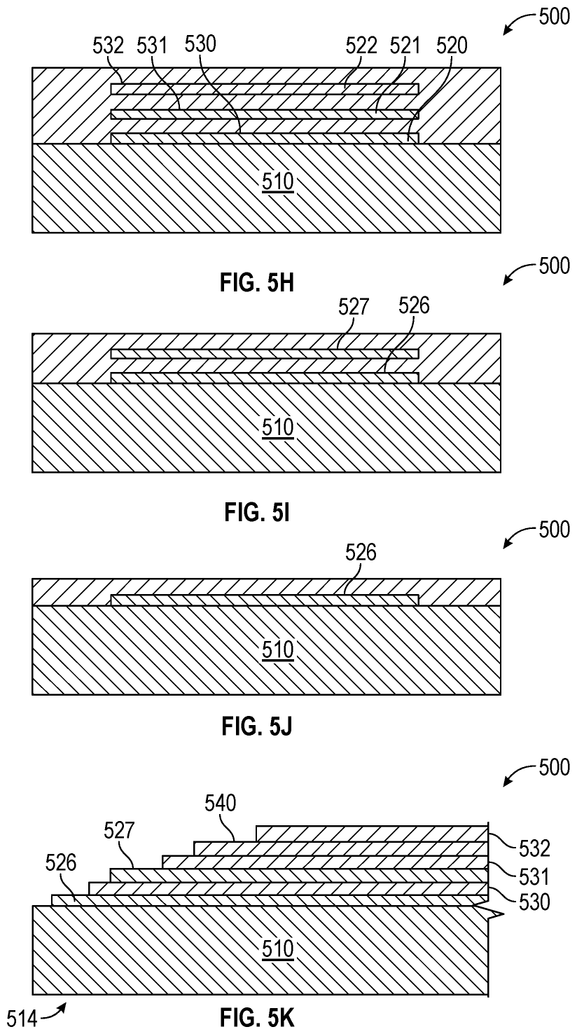

FIGS. 5A-5K illustrate a single sided stacked analyte sensor assembly 500, in accordance with an example. While the sensor assembly 500 is similar to sensor assembly 400, the stacked version in sensor assembly 500 includes multiple insulating and electrode layers on top of the substrate. The sensor assembly 500 can include similar components to those of sensor assembly 400 discussed above, except where otherwise noted.

FIGS. 5A to 5G depict schematic top-down drawings of various layers of the sensor assembly 500. FIGS. 5H to 5K depict schematic cross-section drawings of the full sensor assembly 500.

The sensor assembly 500 can have a first end 512 and a second end 514. The sensor assembly 500 can include polymeric substrate 510, conductive layers 516, 517, 518, conductive traces 520, 521, 522, connector pads 523, 524, 525, working electrode 526, counter electrode 527, insulating layers 530, 532, 534, and reference electrode 540. In sensor assembly 500, a single-sided stacked configuration is used. In the sensor assembly 500, a three-electrode sensor is shown, with a working electrode (WE) 526, a counter electrode (CE) 527 and a reference electrode (RE) 540. In sensor assembly 500, the electrodes are stacked or staggered, and geometrically offset from each other along a z-axis.

In the sensor assembly 500, instead of the co-planar arrangement discussed above, multiple conductive layers 516, 517, 518, and insulating layers 530, 532, 534, are stacked. The conductive layers 516, 517, 518, can be staggered for specified exposure of electrodes 526, 527, 540, and connector pads 523, 524, 525.

Shown in FIG. 5B, the first conductive layer 516 can be deposited onto the polymeric substrate 510 to form a first conductive trace 520, first sensing electrode 526, and first connector pad 524. Shown in FIG. 5C, the first insulating layer 530 can be deposited on top of the first conductive trace 520, leaving the first sensing electrode 526 and the first connector pad 524 exposed. In an example, the first conductive layer 516 on the polymeric substrate 510 can be printed from a conductive ink. In some examples, the first conductive layer 516, or other conductive layers as discussed herein, carbon conductive ink may be suitable for use in biosensor applications. In other examples, other inks including noble metals may be used to form one or more conductive layers discussed herein, including the layers discussed in FIGS. 5A-5K.

In FIG. 5D, the second conductive layer 517 can be deposited over the first insulating layer 530 to for the second conductive trace 521, the second sensing electrode 527, and the second connector pad 525. The second conductive layer 517 can be printed from the same or a different conductive ink used for the first conductive layer 516, for example, a platinum ink suitable for biosensor applications. The second insulating layer 532 can be deposited over the second conductive layer 517, leaving openings 591, 593, for the second sensing electrode 527 and the second connector pad 525.

In some cases, this process can be repeated to form additional sensing electrode and corresponding layers of conductive and insulating material. In sensor assembly 500, a third conductive layer 518 and a third insulating layer 534 are added in FIGS. 5F and 5G. In an example, the third conductive layer 518 can be made of a silver/silver chloride ink, deposited on top of the second insulation layer 532 to form the reference electrode 540. However, additional layers can be added as desired.

FIGS. 5H, 5I, and 5J depict cross-sectional views of the sensor assembly 500 at varying points along its length. In FIG. 5H, a cross-sectional view closer between the first end 512 and the second end 514, the conductive traces 520, 521, 522, and interspersed insulating layers 530, 532, 534, can be seen. Further along the length of the sensor assembly 500, a cross-sectional view of the sensor assembly 500 can be seen in FIG. 5I, with the electrodes 526, 527. The cross-sectional view in FIG. 5J is further towards the second end 514.

FIG. 5K depicts a side view of the second end 514 of the sensor assembly 500. The view on FIG. 5K shows the sensing end of the sensor assembly 500, and helps illustrate the stair-like cross section of such a stacked arrangement. Like the above-mentioned views, the view of FIG. 5K depicts the polymeric substrate 510, with the electrodes 526, 527, 540 stacked and interspersed with the insulating layers 530, 531, 532, such that the electrodes 526, 527, 540, are exposed at various lengths away from the second end 514.

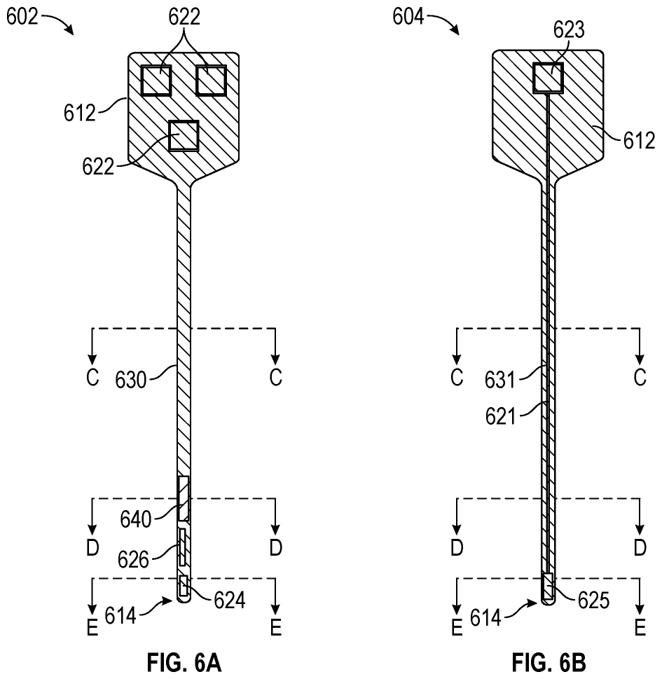

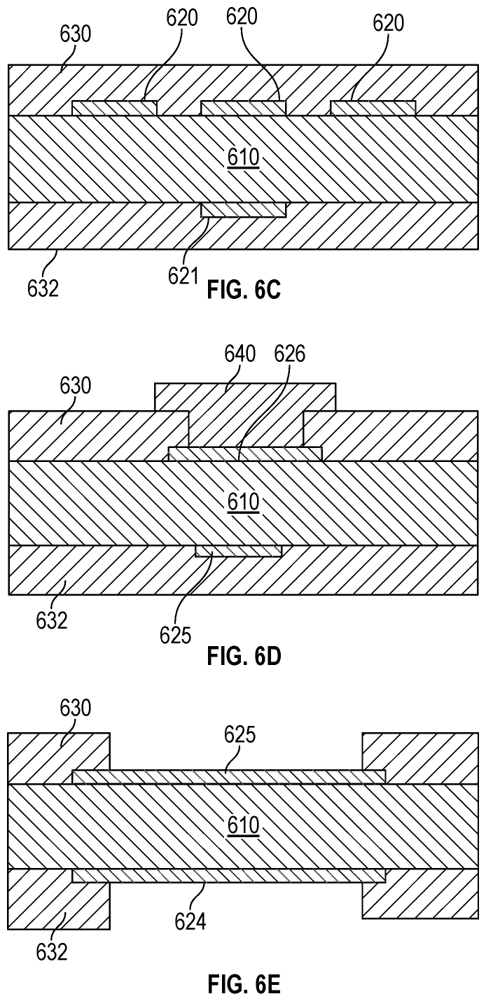

FIGS. 6A-6E illustrate a double-sided co-planar unconnected analyte sensor assembly 600, in accordance with an example. While the sensor assembly 600 is similar to sensor assembly 400, the double-sided version in sensor assembly 600 includes two sides on the substrate. The sensor assembly 600 can include similar components to those of sensor assembly 400 discussed above, except where otherwise noted.

FIGS. 6A to 6B depict schematic top-down drawings of opposing sides of the sensor assembly 600. FIGS. 6C to 6E depict schematic cross-section drawings of the full sensor assembly 600.

The sensor assembly 600 can have a first side 602 and a second side 604 opposite the first side, in addition to a first end 612 and a second end 614. The sensor assembly 600 can include substrate 610, conductive traces 620, 621, connector pads 622, 623 working electrodes 624, 625, counter electrode 626, insulating layers 630, 632, and reference electrode 640. In sensor assembly 600, a double-sided planar configuration is used. In the sensor assembly 600, a multiple-electrode sensor is shown, with two working electrodes (WE) 624, 625, a counter electrode (CE) 626 and a reference electrode (RE) 640. In sensor assembly 600, the electrodes are co-planar. The sensor assembly 600 is an unconnected variation.

In sensor assembly 600, structures can be formed on both sides 602, 604, of the substrate 610. For example, the connector pads 622, 623, can be formed, respectively, on opposing sides 602, 604. This can allow for connection to the sensing electronics from both sides of the sensor assembly 600. Similarly, the conductive traces 620, 621, can be formed on both sides 602, 604, of the sensor assembly 600. On each the first side 602 and the second side 604 the conductive traces 620, 621, can be co-planar with each other.

The insulating layers 630, 632, such as a solder mask or other insulating material, can be deposited over the conductive layers including the conductive traces 620, 621. Openings can be formed in the insulating layers 630, 632, to form the working electrodes 624, 625, and the counter electrode 626. An opening can be left for the reference electrode 640. A reference electrode material, such as silver/silver chloride, can be deposited on the designated sensing surface for the reference electrode 640. The insulating material can include epoxy, polyimide, polyurethane, polyethylene, or other materials or combinations of materials.

As illustrated in FIGS. 6A to 6E, the double-sided sensor assembly 600 can include a first working electrode 624, a second working electrode 625, a counter electrode 626, and a reference electrode 640. In some cases, such a double-sided sensor can contain more or less electrodes. For example, a double-sided sensor can include a single working electrode and a reference electrode.

FIGS. 6C to 6E depict cross-sections of the sensor assembly 600. Shown in FIG. 6C is a cross section along line C-C, where the substrate 610 is situated between the two insulating layers 630, 632. The substrate 610 can be, for example, about 50 microns thick. Conductive traces 620, 621, can be seen. On the first side 602, three conductive traces 620 extend along the length of the sensor assembly 600, each connecting to a connector pad 622. The conductive traces 621 on the second side 604 can connect to the connector pad 623.

In FIG. 6D, the cross-section is taken along line D-D. The reference electrode 640 can be seen at this point. In FIG. 6E, the cross-section is taken along line E-E, both working electrodes 624, 625, can be seen on opposing sides 602, 604, of the sensor assembly 600.

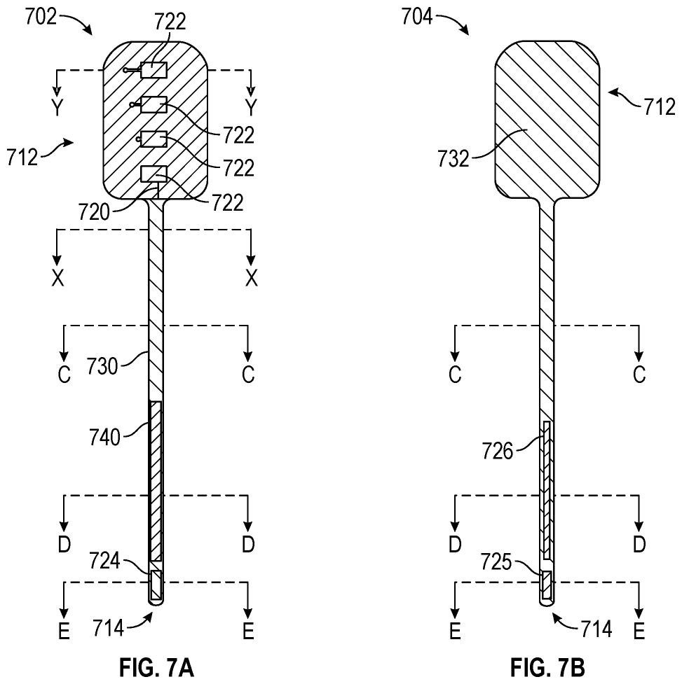

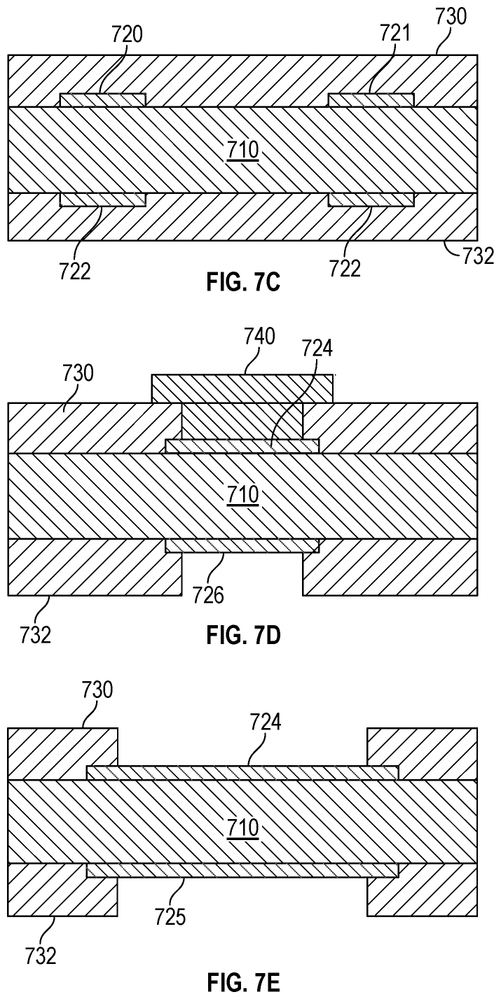

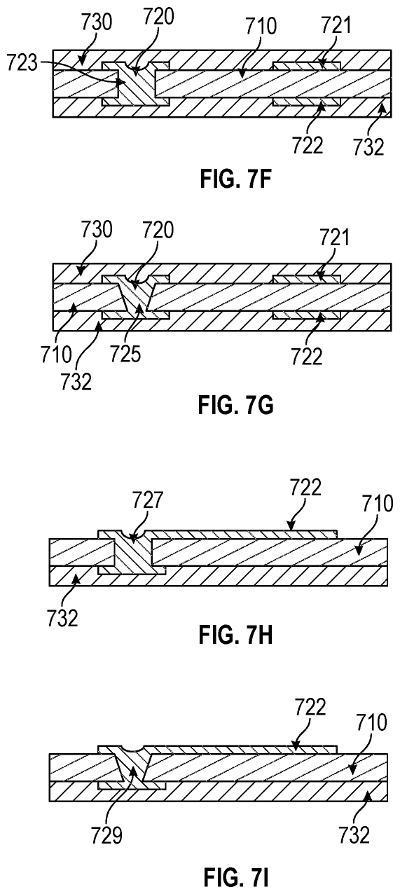

FIGS. 7A-7E illustrate a double-sided co-planar connected analyte sensor assembly 700, in accordance with an example. While the sensor assembly 700 is similar to sensor assembly 400, the double-sided version in sensor assembly 700 includes two sides on the substrate. The sensor assembly 700 can include similar components to those of sensor assembly 400 discussed above, except where otherwise noted.

FIGS. 7A to 7B depict schematic top-down drawings of opposing sides of the sensor assembly 700. FIGS. 7C to 7E depict schematic cross-section drawings of the full sensor assembly 700. In some cases, the sensor assembly 700 can include a chamfer end, a rounded end, a flat end, or other appropriate shape.

The sensor assembly 700 can have a first side 702 and a second side 704 opposite the first side, in addition to a first end 712 and a second end 714. The sensor assembly 700 can include substrate 710, conductive traces 720, 721, connector pads 722, working electrodes 724, 725, counter electrode 726, insulating layers 730, 732, and reference electrode 740. In sensor assembly 700, a double-sided planar configuration is used. In the sensor assembly 700, a multiple-electrode sensor is shown, with two working electrodes (WE) 724, 725, a counter electrode (CE) 726 and a reference electrode (RE) 740. In sensor assembly 700, the electrodes are co-planar. The sensor assembly 700 is a co-planar, connected variation.

In sensor assembly 700, the substrate 710 is situated between two sides 702, 704, which can each host several co-planar components. For example, co-planar conductive traces 720 can be on the first side 702, and second conductive traces 721 can be on the second side 704. Each side 702, 704, can be covered by an insulating layer 730, 732. The insulating layers 730, 732, can define electrodes 724, 725, 726, and an area for the reference electrode 740.

The sensor assembly 700 can also include via, which can provide for an electrical connection between both sides 702, 704 of the sensor assembly 700. Including vias can allow for connection to the sensing electronics through the connector pads 722 on a single side, such as first side 702, of the sensor, as well as routing traces to new locations, allowing flexible geometries to be used. The vias can be formed from various conductive materials discussed herein, including carbon, graphitic carbon, Pt, or combinations including Pt and C, Au and C. In some examples, the conductive material forming the vias between sides 702, 704 of the assembly or other assemblies as discussed herein may or may not further include conductive nanoparticles.

Shown in FIGS. 7A to 7E, the sensor assembly 700 can include four connector pads 722 can be on a first side 702, electrically coupled to the electrodes 725, 740, on the second side 704 by vias and traces. In some cases, a WE, RE, and CE can be placed on the opposite side of the sensor assembly 700 to the connector pads 722. In some cases, as shown in sensor assembly 700, a first working electrode 724 and counter electrode 726 can be located on the first side 702 of the sensor, while a second working electrode 725 and a reference electrode 740 can be located on the other side 704 of the sensor. Vias can be used to establish electrical contact between traces and pads on both sides 702, 704 of the sensor assembly 700, since the connector pads 722 for connecting to the sensing electronics are located only on one side.

While the sensor assembly 800 is similar to sensor assembly 700, the double-sided version in sensor assembly 800 includes two sides on the substrate. The sensor assembly 800 can include similar components to those of sensor assembly 500 discussed above, except where otherwise noted.

The sensor assembly 800 can have a first side 802 and a second side 804 opposite the first side, in addition to a first end 812 and a second end 814. The sensor assembly 800 can include substrate 810, conductive traces 820, 821, connector pads 822, working electrodes 824, 825, counter electrode 826, insulating layers 830, 832, and reference electrode 840. In sensor assembly 800, a double-sided stacked configuration is used. In the sensor assembly 800, a multiple-electrode sensor is shown, with two working electrodes (WE) 824, 825, a counter electrode (CE) 826 and a reference electrode (RE) 840. In sensor assembly 800, the electrodes are staggered.

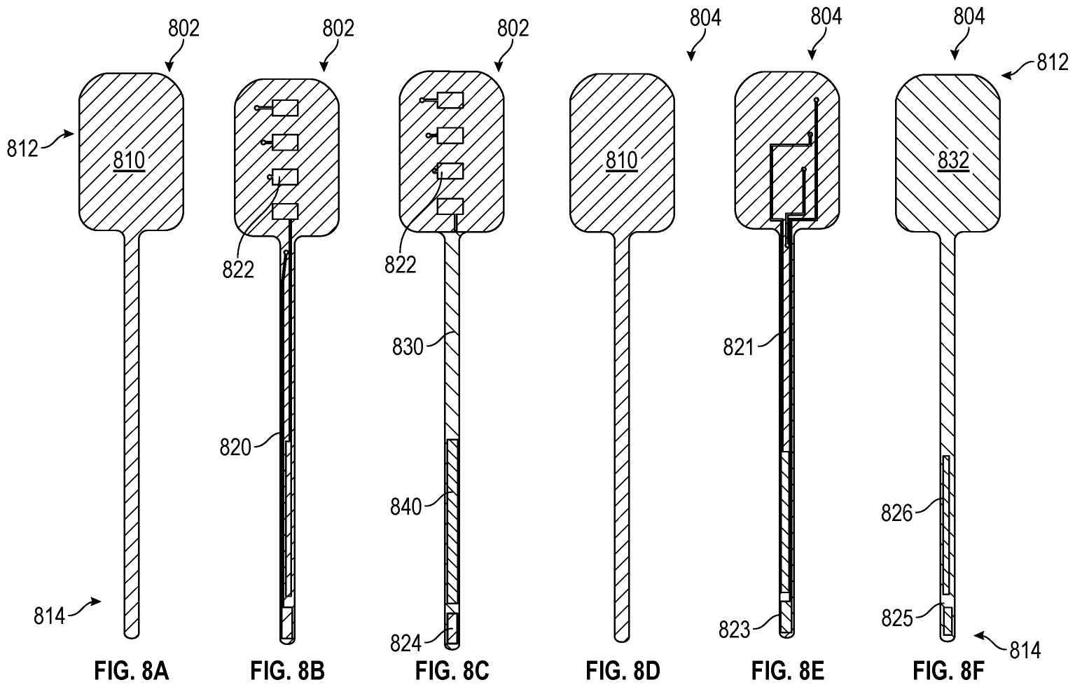

The sensor assembly 800 is similar to the sensor assembly 400 above, but with two sides. FIGS. 8A-8F illustrate a double-sided connected analyte sensor assembly 800, in accordance with an example. FIGS. 8A-8C depict various layers of a first side 802, while FIGS. 8D-8F depict various layers of a second side 804.

FIG. 8A depicts the substrate 810 on first side 802. FIG. 8B depicts deposition of a conductive layer on the substrate to form the connector pads 822, conductive traces 820, first working electrode 824, and the counter electrode 826. FIG. 8C depicts deposition of the insulating layer 830 on the first side 802.

FIG. 8D depicts the substrate 810 on second side 804. FIG. 8E depicts deposition of a conductive layer on the substrate to form the conductive traces 821, and the second working electrode 825. FIG. 8E depicts building up of the second insulating layer 832, and the reference electrode 840.

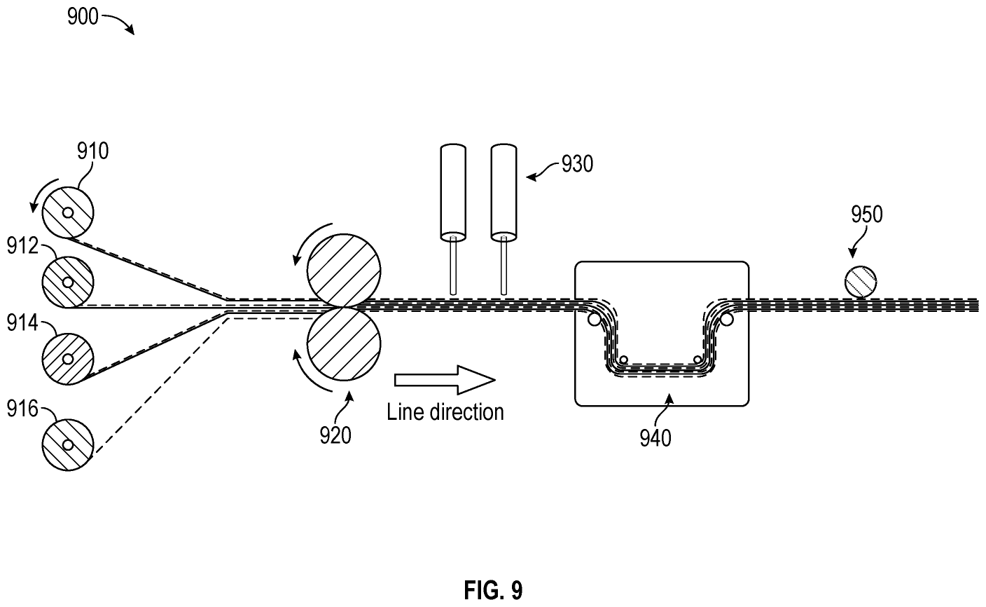

FIGS. 9 to 14 show various examples of methods of manufacturing planar sensor assemblies, such as the sensor assemblies 500 to 800 discussed above.

FIG. 9 illustrates a schematic of a system 900 for making a co-planar analyte sensor assembly, in accordance with an example. The system 900 can use a variety of components including layer spools 910, 912, 914, 916, lamination tool 920, laser skiving station 930, electroplating bath 940, and rotogravure 950. In other examples, the electrodes discussed herein may be formed via a micro-dispensing operation (not shown here) subsequent to laser skiving at the laser skiving station 930. In this example, the micro-dispensing operation may be followed by laser singulation as discussed herein.

In the example of FIG. 9, components may move from left to right. Production can be continuous and scalable.

First, layer spools 910, 912, 914, 916, are fed into the system 900. The first layer spool 910 can include the substrate, such as the thin polymeric substrates discussed above. The second layer spool 912 can, for example, include a first conductive layer, a first insulating layer, and an adhesive. The third layer spool 914 can include a second conductive layer, a second insulating layer, and an adhesive. The fourth layer spool 916 can include a third conductive layer, a third insulating layer, and an adhesive.

The layer spools 910, 912, 914, 916 can be aligned, such as to alternate conductive layers and insulating layers. The layer spools 910, 912, 914, 916 can be fed into the system 900 and into the lamination tool 920, where the layers can be laminated together to produce a planar sheet with multiple layers being fed through the production line. In some examples, the conductive layers formed via lamination operations may be formed using a thin metal foil. The thin metal foils discussed herein may vary in thickness depending upon the application and the equipment employed to perform the lamination operations. The thin metal foil may include nickel, titanium, gold, platinum, or alloys or combinations thereof. In other examples, the conductive layers formed via lamination operations as discussed herein may include a conductive material deposited on to a thin polymeric layer. Depending upon the example, the conductive material may include carbon nanotubes or nanoparticles, polyaniline, or poly(3,4-ethylenedioxythiophene) polystyrene sulfonate (PEDOT:PSS).

In other examples, the third layer spool 914 includes, in addition to or instead of a second conductive layer, a second insulating layer, and an adhesive, another substrate which may or may not be of the same composition and thickness as the substrate of the first layer spool 910.

The planar sheet can continue along the production line to the laser skiving station 930, where one or more lasers can be used to open contact pads and electrodes in the sensors. Additionally, the planar sheet can be cut up and singulated (e.g., a singulation operation is performed) to produce individual sensor assemblies.

Optionally, the individualized sensor assemblies can be put through an electroplating bath 940 to allow for electrode metallization. In some cases, the individualized sensor assemblies can be put through the rotogravure 950 to allow for reference electrode deposition. In some cases, discrete dispensing methods or slot die coatings can be used instead of or in addition to the above.

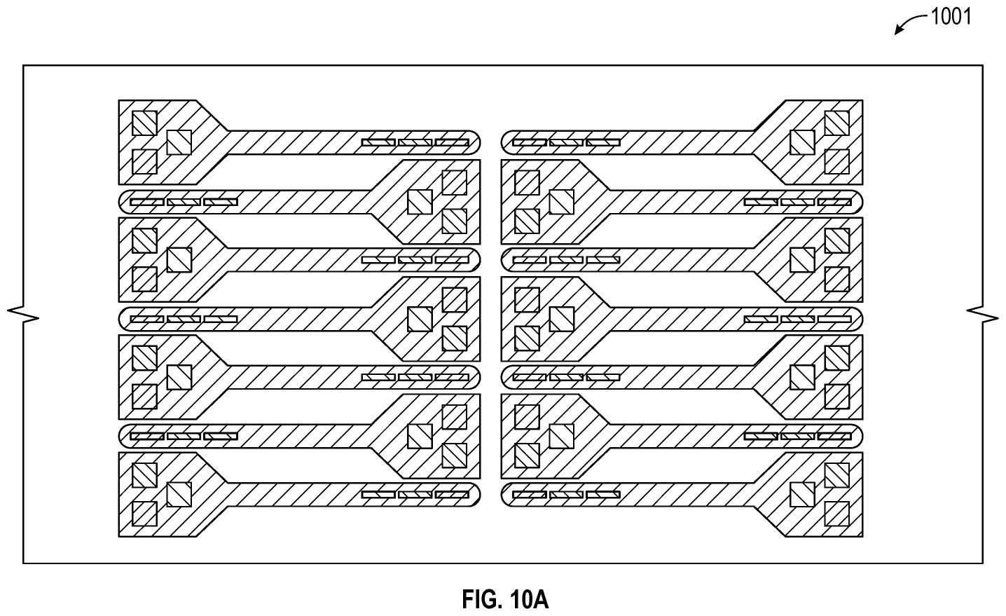

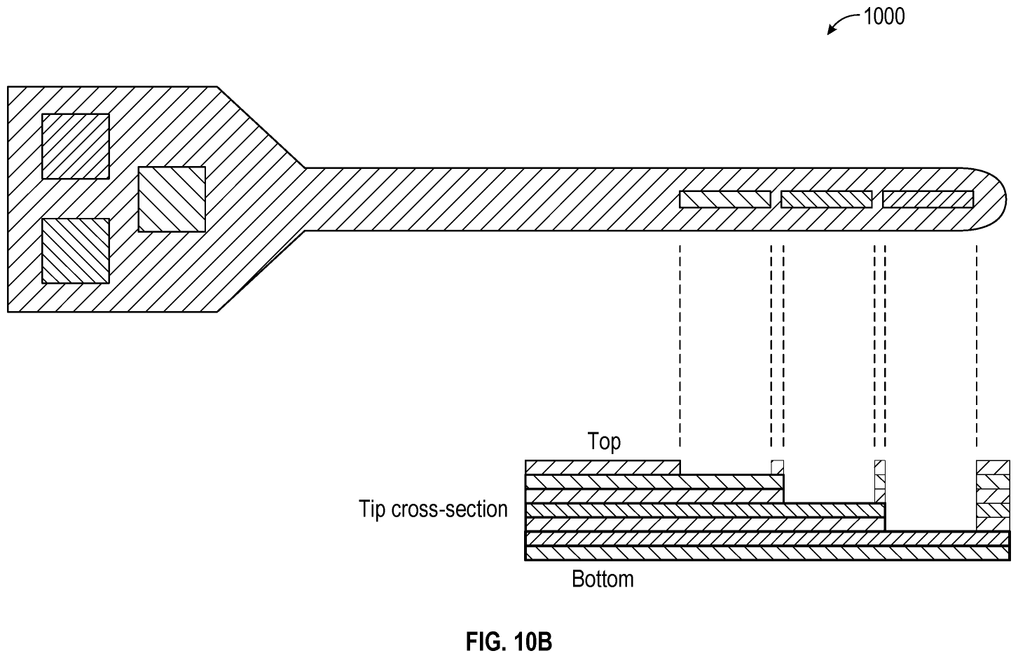

FIGS. 10A-10B illustrate a method of making multi-layer co-planar analyte sensor assemblies, in accordance with an example. Shown in FIG. 10A, a plurality of individual sensor assemblies 1000 are produced together on a production line in sheet 1001, such as by the methods discussed above with reference to FIG. 9. The sheet 1001 can be moved along the production line and the individual sensor assemblies 1000 can be separated, such as by skiving or other cutting methods.

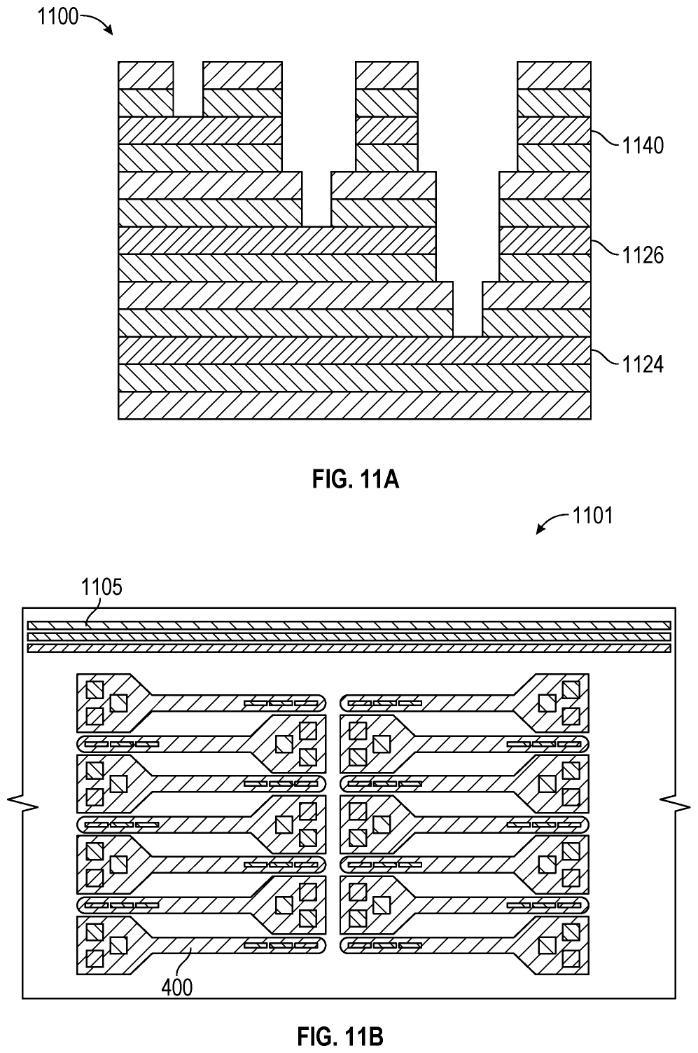

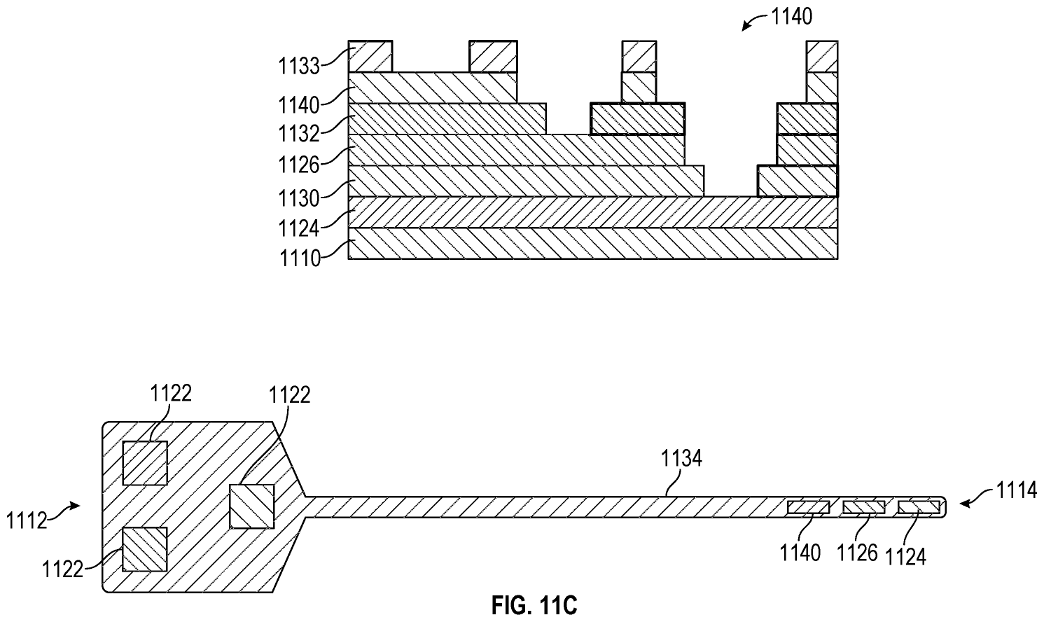

FIGS. 11A-11C illustrate a method of making a co-planar analyte sensor assembly 1100, in accordance with an example. Shown in FIG. 11A, on a substrate 1110, electrodes 1124, 1126, and 1140 can be layered in a stack, alternating with insulating layers 1130, 1132, 1133. The layers can, for example, be laminated together.

Shown in FIG. 11B, a plurality of sensor assemblies can be on a feed planar sheet 1101 moving along the assembly line. Here, the individual sensor assembly electrode can be opened, and electrodeposition can occur (1105) as desired. The individual assemblies can be separated out, such as by laser skiving.

Shown in FIG. 11C, the completed individual assembly 1100 can include first electrode 1124, second electrode 1126, and third electrode 1140, stacked on top of each other and separated by insulating layers 1130, 1132, 1134. The assembly 1100 can be hosted by a substrate 1110.

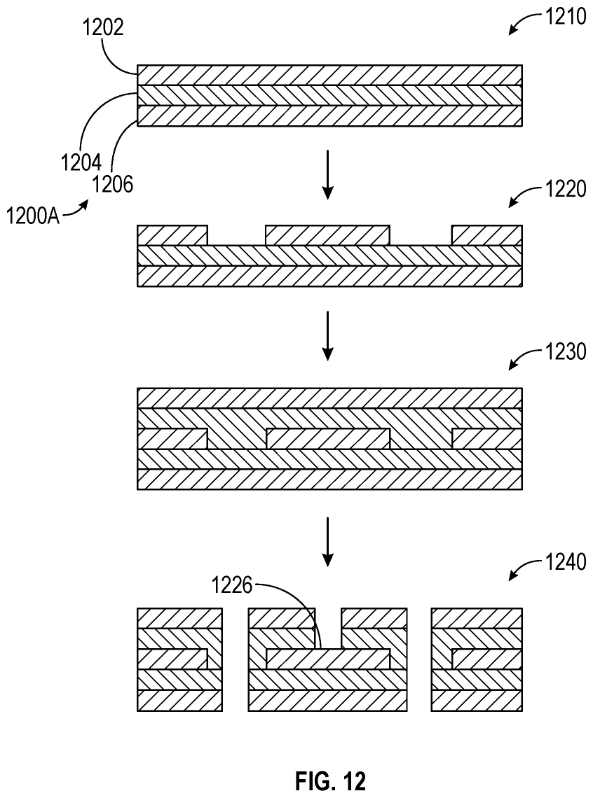

FIG. 12 illustrates a method 1200 of making a co-planar analyte sensor assembly, in accordance with an example. At operation 1210 a substrate 1206, first adhesive layer 1204, and first electrode 1202 can be laminated together. Next, at 1220, the electrode can be exposed, such as through laser skiving. At operation 1230, a second insulating layer can be applied and laminated onto the first electrode 1202, along with a top layer. The assembly can be further skived at operation 1240 to expose the electrode 1226.

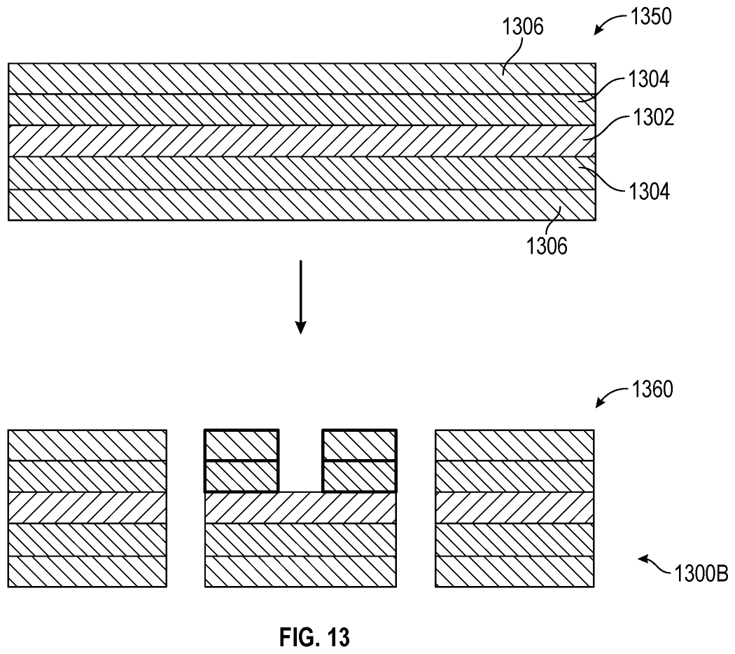

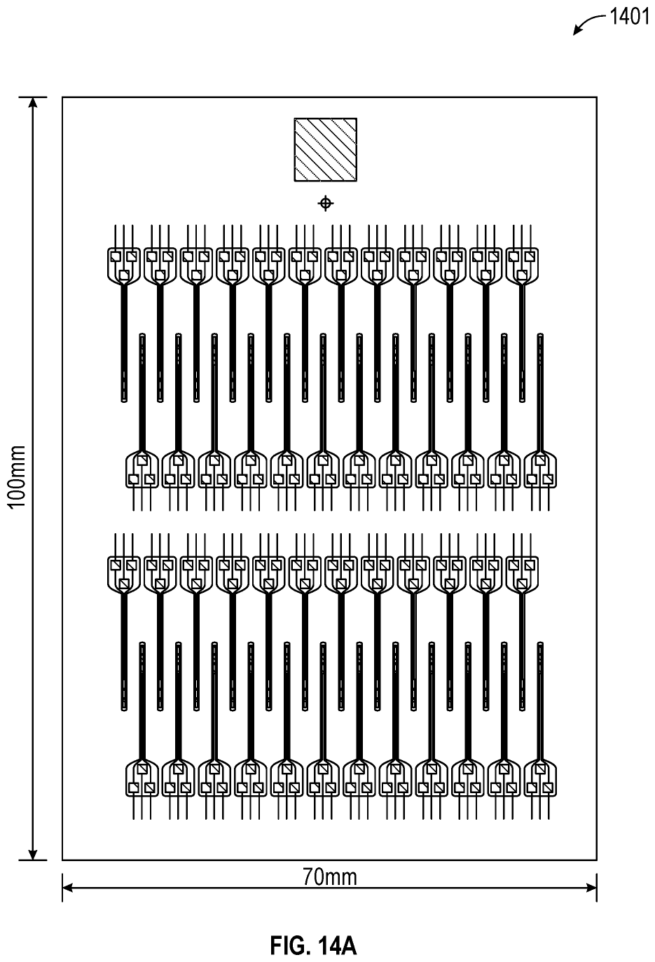

FIG. 13 illustrates a method 1300 of making a co-planar analyte sensor assembly, in accordance with an example. In method 1300, an electrode 1302 can be layered with adhesive layers 1304 and substrate layers 1306 and laminated (operation 1350). In contrast to method 1200, operation 1350 can be done all at once with more layers. Then, at step 1360, the electrode can be revealed, such as through skiving or other specific removal of material.

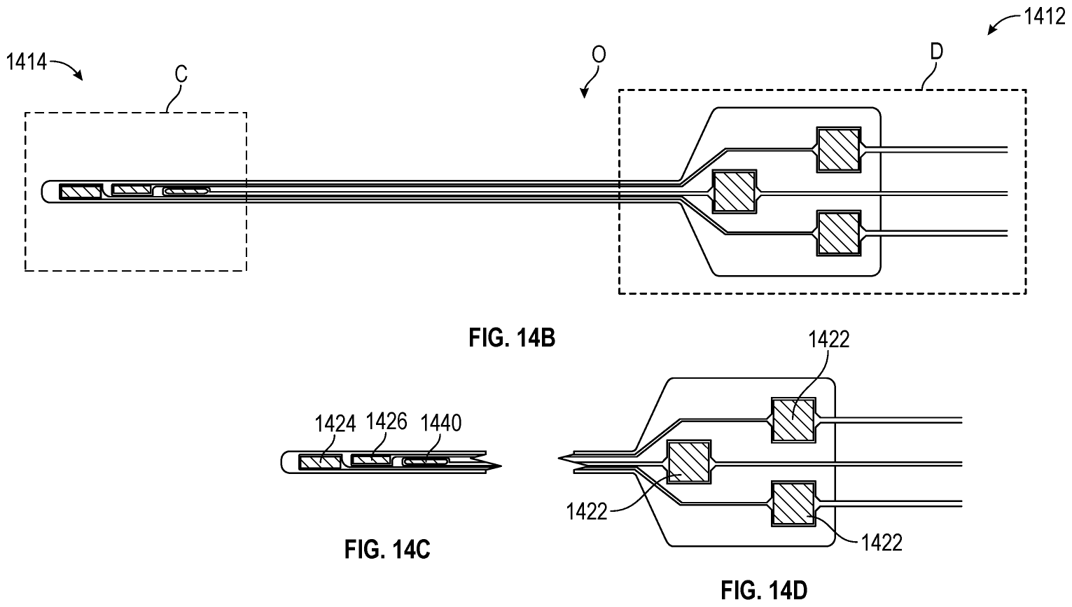

FIGS. 14A-14D illustrate a method of making a skiving co-planar analyte sensor assembly, in accordance with an example. The planar sheet 1401 shown in FIG. 14A can be produced by any of the methods discussed above. The planar sheet 1401 can host a plurality of sensor assemblies 1400. These can be separated, such as by skiving or other separation methods, to individual sensor assemblies 1400.

Shown in FIGS. 14B-14D, the individual sensor assemblies 1400 can each include a first end 1412 and a second end 1414. At the first end 1412, the various connector pads 1422 can be opened, such as by skiving, to allow for electrical connection. Similarly, at the second end, the electrodes 1424, 1426, and 1440, can be opened, such as by skiving, to allow for sensing.

### Coaxial Analyte Sensors

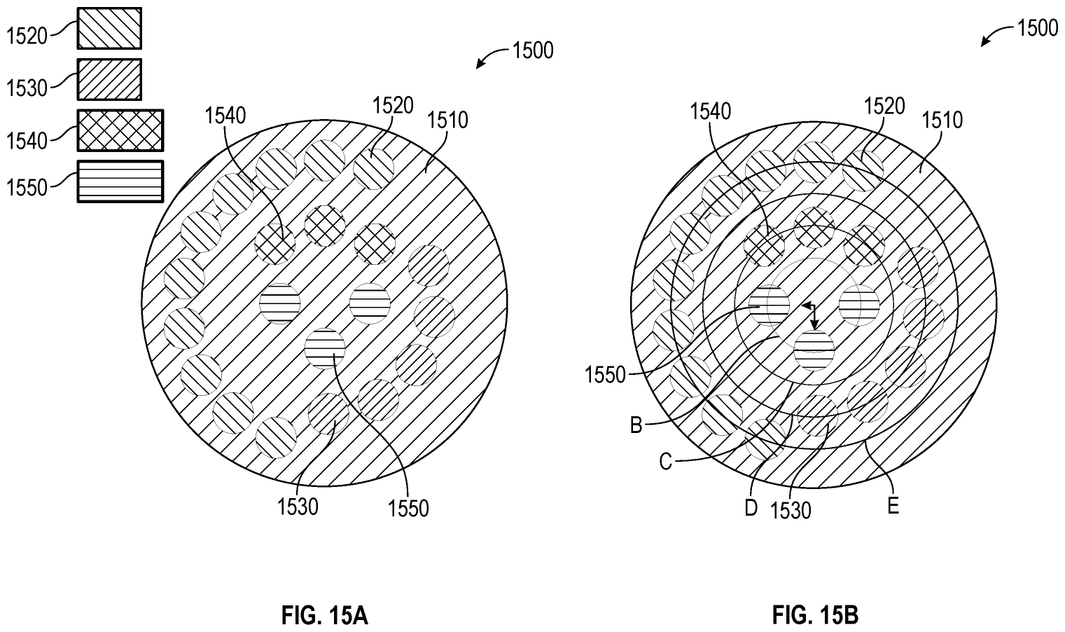

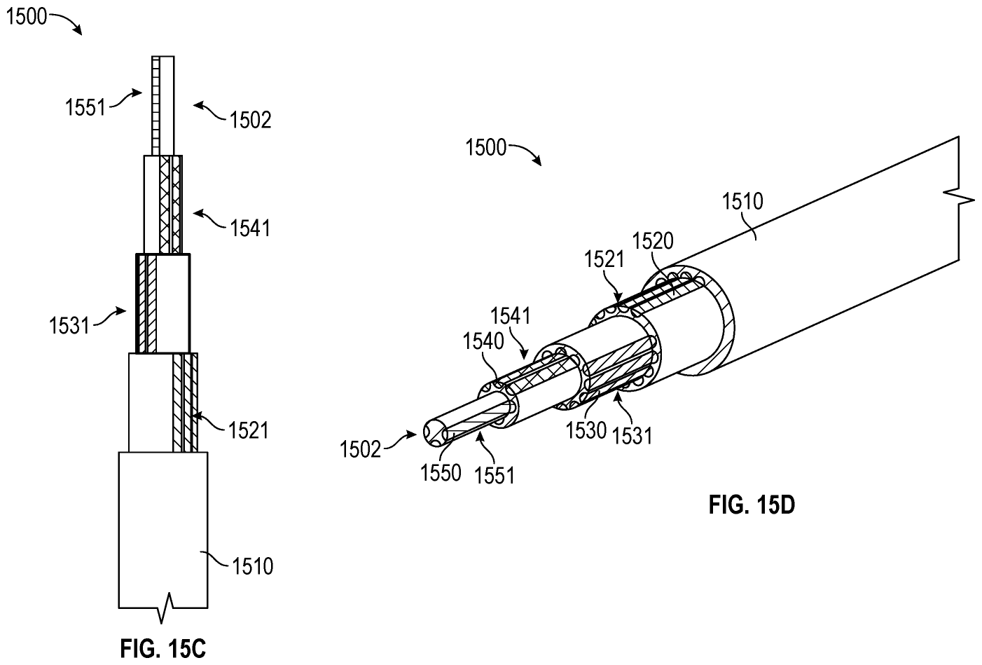

FIGS. 15A-20B depicts example coaxial sensor assemblies. For each of these examples, the sensor substrates can be cylindrical, and can be manufactured at least in part through one or more extrusion operations. Multiple coating layers can be used with these sensors in the sensing regions to allow proper function of the sensors themselves. In some cases, selective sensing chemistries can be used to coat the sensing regions. Those chemistries can include, for example, enzymes, mediators, or layers, such as to prevent interference from highly charged small molecules. Such layers can also limit the flux of analytes to the sensors. As discussed in more detail below, these layers can be applied using a variety of methods, such as discrete dispensing, spray coating, dip coating, or other methods. Various electrode configurations may be discussed herein.

Each of the coaxial sensor assemblies discussed below can, in some cases, have a diameter of about 100 micrometers (μm) to about 300 micrometers. In some cases, narrower diameters can be used. In some cases, wider diameters can be used. The sensor assemblies can be of varying lengths, depending on the desired implantation/insertion depth and particular sensor connections. The coaxial sensor assemblies can, for example, include two-electrode configurations, or three-electrode configurations. The sensor substrates can be singulated into individual sensors prior to application of coating layers. In an example, the coaxial sensor assemblies can include a substrate including a cylindrical member having a central axis ringed by a plurality of concentric circles of increasing radii, the concentric circles visible by a cross section of the substrate. In some cases, electrodes can include a cylindrical shell extending along the concentric circles. In some cases, electrodes can include wires extending along one of the plurality of concentric circles offset from each other by a non-zero angle. In some cases, the electrodes can be nested within each other.

FIGS. 15A-15D illustrate a coaxial analyte sensor assembly 1500, in accordance with an example. The sensor assembly 1500 can include a polymer substrate 1510, a first electrode 1520, a second electrode 1530, a third electrode 1540, and a fourth electrode 1550. In one example, the electrodes (1520, 1530, 1540, 1550) may be configured as a counter electrode 1520, reference electrode 1530, first working electrode 1540, and second working electrode 1550. In another example, the electrodes (1520, 1530, 1540, 1550) may be configured as a reference electrode 1520, counter electrode 1530, first working electrode 1540, and second working electrode 1550.

The sensor assembly 1500 can extend from a first end 1502 to a second end. The first end 1502 can be a sensing end, such as for implantation into a patient for sensing an analyte therein. The second end can be for electrical connection.

The polymer substrate 1510 can be an insulating polymer, such as polyurethane or another suitable insulating matrix. The polymer substrate 1510 can extend from the first end 1502 to the second end, and host a plurality of wires, each corresponding to one of the electrodes. Each of the counter electrode 1520, reference electrode 1530, first working electrode 1540, and second working electrode 1550, can be embedded within the insulating polymer substrate 1510.

The electrodes 1520, 1530, 1540, 1550, can be made of round wires of a suitable conductive material for electrochemical sensing, for example, platinum, gold, silver, silver, or chloride-coated silver. The wires can extend laterally along the polymer substrate 1510. The electrodes 1520, 1530, 1540, 1550, can respectively have sensing areas 1521, 1531, 1541, 1551 for electrical connection and sensing when the sensor assembly 1500 is connected to a patient for sensing analytes. In the sensor assembly 1500, the electrode 1520, 1530, 1540, 1550, can be changed or altered to be working electrodes, counter electrodes, and reference electrodes as desired for the specific use.

In the example of FIGS. 15A-15D, the various wires can be patterned around a central axis A in the polymer substrate 1510. In some cases, the pattern can be aligned about concentric circles, particular axes, a spiral pattern, or other patterns as desired. The electrode 1520, 1530, 1540, 1550 wires can pattern co-axially in such a way to allow for orientation-agnostic connection as the electrodes 1520, 1530, 1540, 1550 are exposed. Shown in FIG. 15B, the various types of electrodes can each be aligned with concentric circles B, C, D, and E, forming a faux spiral when viewed in a cross-section.

The sensor assembly 1500 can be made by co-extruding the suitable conductive materials with the insulating polymer of the substrate. To form the electrodes 1520, 1530, 1540, 1550 sensing areas, 1521, 1531, 1541, 1551, insulating material (and conductive material) can be removed in selective areas to expose particular electrodes and shown the sensing areas 1521, 1531, 1541, 1551. The exposed sensing areas 1521, 1531, 1541, 1551 can be seen in FIGS. 15C and 15D. Material can be removed to expose the sensing areas of the electrodes 1520, 1530, 1540, 1550, for example, by laser ablation. In sensor assembly 1500, a step-like structure can be used to expose sensing areas for the electrodes 1520, 1530, 1540, 1550. In some cases, the sensing tip could take on additional shapes, such as conical. In some cases, the arrangement of the electrodes 1520, 1530, 1540, 1550 can be varied so the locations of the electrodes are different.

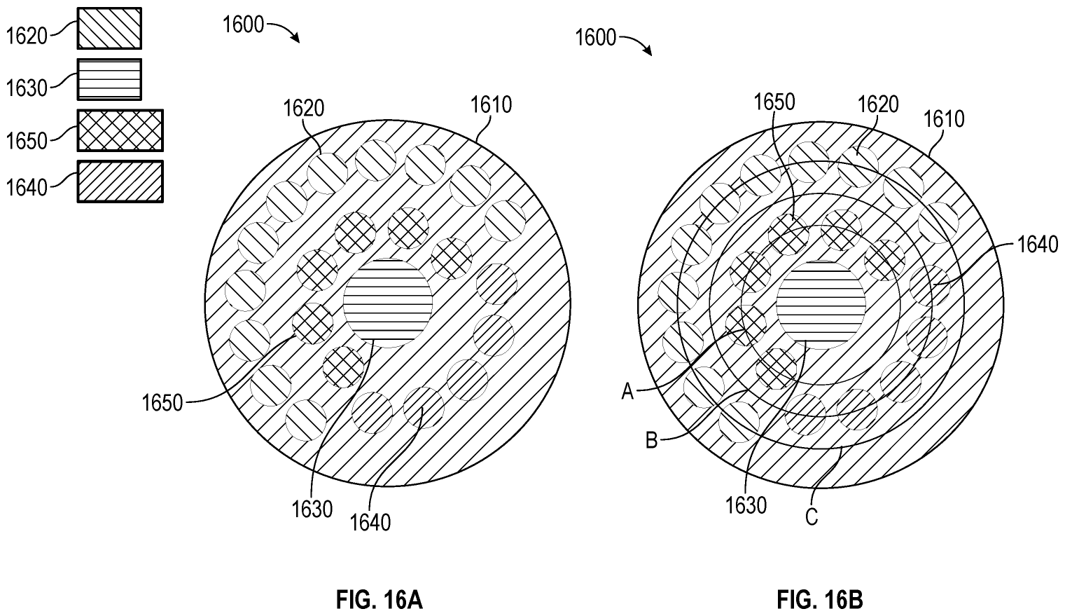

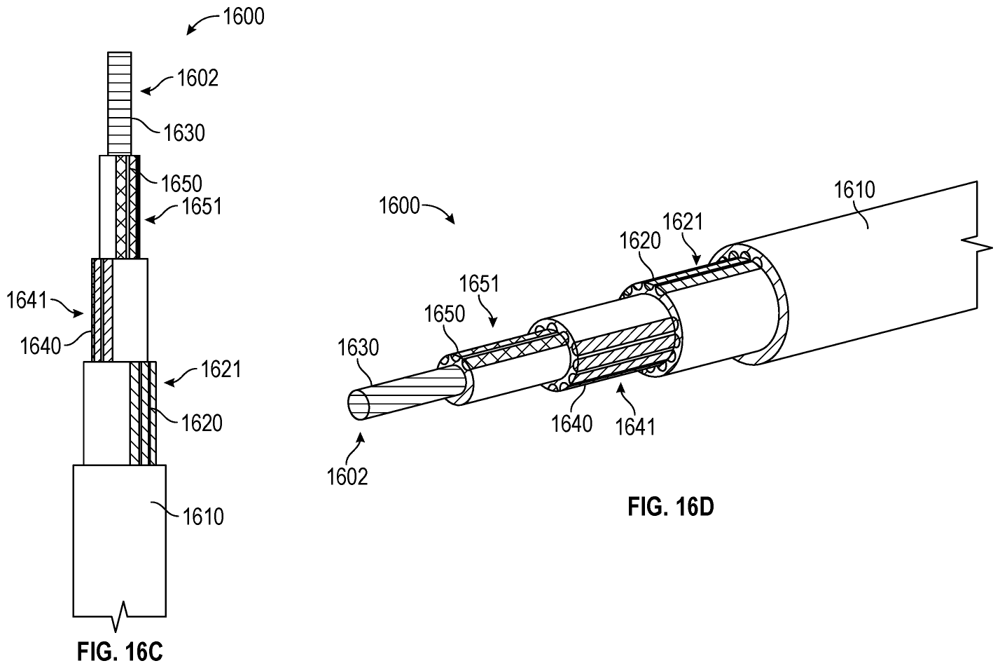

FIGS. 16A-16D illustrate a coaxial analyte sensor assembly 1600 having radial wires in an insulating polymer, in accordance with an example. The sensor assembly 1600 can include an insulating polymer substrate 1610, a first electrode 1620, a second electrode 1630, a third electrode 1640, and a fourth electrode 1650. In one example, the electrodes (1620, 1630, 1640, 1650) may be configured as a counter electrode 1620, reference electrode 1630, first working electrode 1640, and second working electrode 1650. In another example, the electrodes (1620, 1630, 1640, 1650) may be configured as a reference electrode 1620, counter electrode 1630, first working electrode 1640, and second working electrode 1650. The sensor assembly 1600 can extend from a first end 1602 to a second end. The first end 1602 can be a sensing end, such as for implantation into a patient for sensing an analyte therein. The second end can be for electrical connection. The components of sensor assembly 1600 can be similar to those in sensor assembly 1500 discussed above, except where otherwise noted.

In sensor assembly 1600, the reference electrode 1630 can extend along a central axis A. The reference electrode 1630 can serve as a central portion of the sensor assembly 1600. The reference electrode 1630 can include a single wire extruded as the core of the assembly. The working electrodes 1640, 1650, and the counter electrode 1620, can still contain multiple wires patterned around the core reference electrode 1630. The electrodes 1620, 1630, 1640, 1650, can respectively have sensing areas 1621, 1631, 1641, 1651 for electrical connection and sensing when the sensor assembly 1600 is connected to a patient for sensing analytes.

Here, the core reference electrode 1630 can be co-extruded with the working electrodes 1640, 1650, and the counter electrode 1620 in the insulating polymer substrate 1610. The core reference electrode 1630 can be, for example, a silver chloride-coated silver wire. The working electrodes 1640, 1650, and the counter electrode 1620 can, for example, be made of a platinum or other conductive material. In some examples, one or more materials used to form the conductive layers discussed herein may include one or more types of conductive nanoparticles. The working electrodes 1640, 1650, and the counter electrode 1620 can be arranged around the core reference electrode 1630 in arcs, such as along concentric circles B, C, and D. This can be seen, for example, in cross-section in FIGS. 16A and 16B.

Similar to sensor assembly 1500, sensing areas 1621, 1631, 1641, and 1651, can be exposed by removal of insulating polymer substrate 1610 in selected areas. Material can be removed, for example, by laser ablation. The sensing areas 1621, 1631, 1641, and 1651, can be seen in FIGS. 16C and 16D. Shown in sensor assembly 1600, the sensing areas 1621, 1631, 1641, and 1651 can be formed in step-like structure. In some cases, other patterning can be used, and/or the locations of various electrodes can be varied.

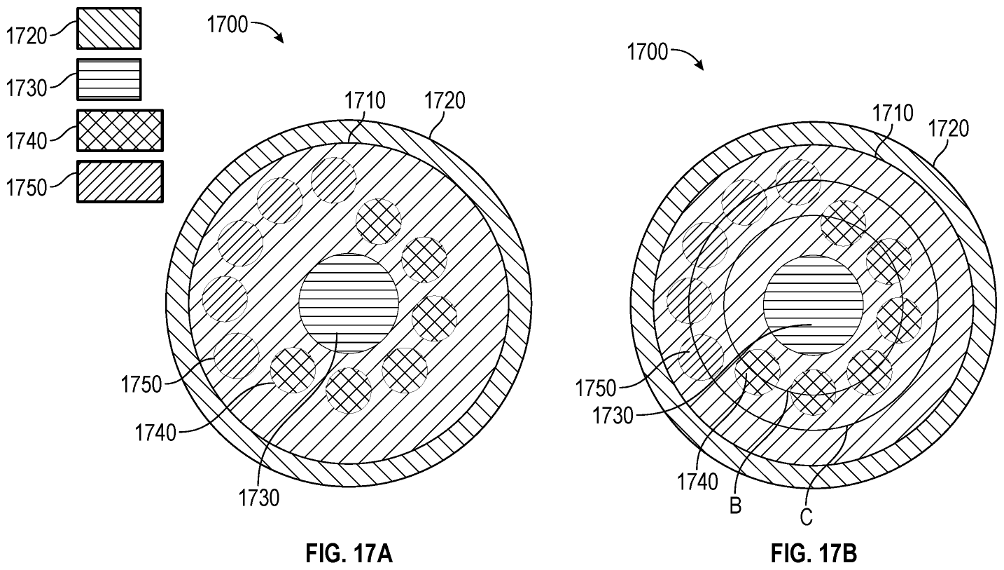

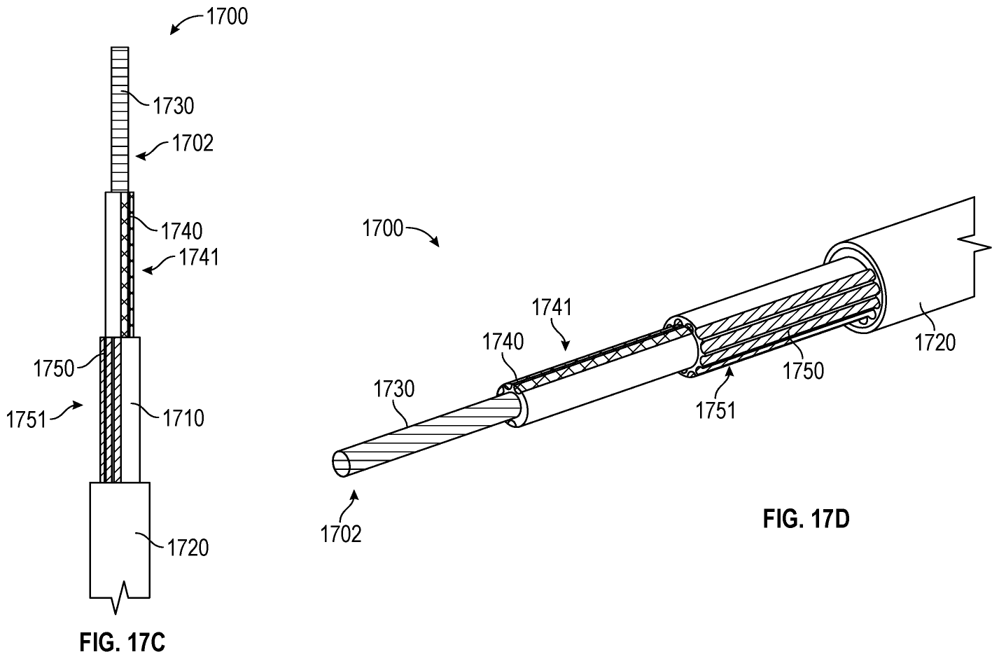

FIGS. 17A-17D illustrate a coaxial analyte sensor assembly 1700 having radial wire electrodes in an insulating polymer substrate 1710, in accordance with an example. The sensor assembly 1700 can include an insulating polymer substrate 1710, a first electrode 1720, a second electrode 1730, a third electrode 1740, and a fourth electrode 1750. In one example of the coaxial analyte sensor assembly 1700, the electrodes (1720, 1730, 1740, 1750) may be configured as a reference electrode 1720, counter electrode 1730, first working electrode 1740, and second working electrode 1750. In another example, the electrodes (1720, 1730, 1740, 1750) may be configured as counter electrode 1720, reference electrode 1730, first working electrode 1740, and second working electrode 1750. The electrodes 1720, 1730, 1740, 1750, can respectively have sensing areas 1721, 1731, 1741, 1751 for electrical connection and sensing when the sensor assembly 1700 is connected to a patient for sensing analytes. The sensor assembly 1700 can extend from a first end 1702 to a second end. The first end 1702 can be a sensing end, such as for implantation into a patient for sensing an analyte therein. The second end can be for electrical connection. The components of sensor assembly 1500 can be similar to those in sensor assembly 1500 discussed above, except where otherwise noted.

In sensor assembly 1700, the counter electrode 1720 can be formed differently from the extrusion of the other electrodes 1730, 1740, 1750. The core reference electrode 1720 and the working electrodes 1740, 1750, can be made by extrusion as discussed above with reference to sensor assemblies 1500 and 1600. However, counter electrode 1720 can be made by coating the insulating polymer substrate 1710, such as with a conductive formulation over the co-extruded electrodes 1730, 1740, 1750 and insulating polymer substrate 1710. Coating can be done by, for example, die coating, spray coating, pad printing, deposition, or other methods.

To form the sensing areas 1731, 1741, 1751, selective portions of the insulating polymer substrate 1710 can be removed, such as by laser ablation. This can be done in a step-like fashion, or in other shapes, such as in a conical shape. The arrangement of the electrodes can additionally be varied.

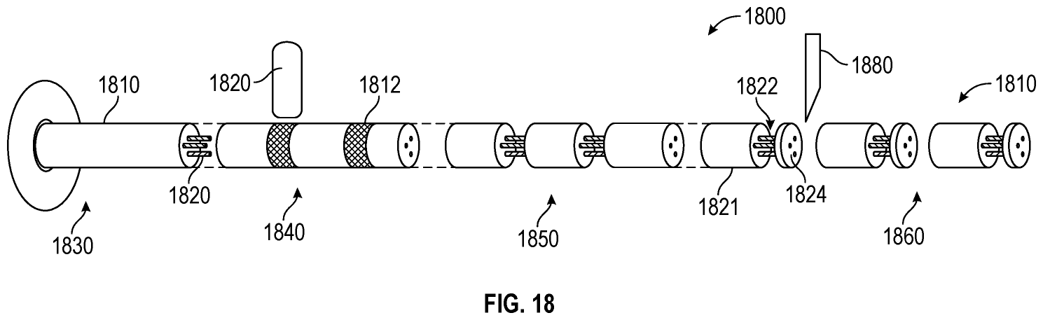

FIG. 18 illustrates a coaxial analyte sensor assembly 1800 including wires embedded in a photoresist insulating polymer, in accordance with an example. Sensor assembly 1800 can include a photoresist insulating polymer substrate 1810 with photoresist portions 1812, and wire electrodes 1820. Shown in FIG. 18 is a method of making individual sensor assemblies 1801 through operations including extrusion operation 1830, UV exposure operation 1840, resist development operation 1850, and singularization operation 1860. During production, a UV light 1870 and a slicing tool 1880 can be used. In this concept, wire electrodes 1820 suitable for electrochemical sensing can be co-extruded with a positive photoresist to form the insulation layer.

Starting at the left of FIG. 18, the photoresist insulating polymer substrate 1810 can be co-extruded with the wire electrodes 1820 at operation 1830. The photoresist insulating polymer substrate 1810 can contain one or more photoresist portions 1812. After extrusion, the UV light 1870 can be used to expose the photoresist portions 1812 and remove a predetermined portion of the material surrounding the wire electrodes 1820. In this case, the extruded photo resistive material with wire electrodes 1820 can be exposed to UV light at the specific locations to define the sensing electrode areas 1822 and a contact area 1824.

The exposed electrode areas 1822 can then be developed and cured, such as with a hard-bake to cure the resist (operation 1850). Individual assemblies 1821 can then be sliced with a mechanical slicing tool 1880 to produce individual sensors.

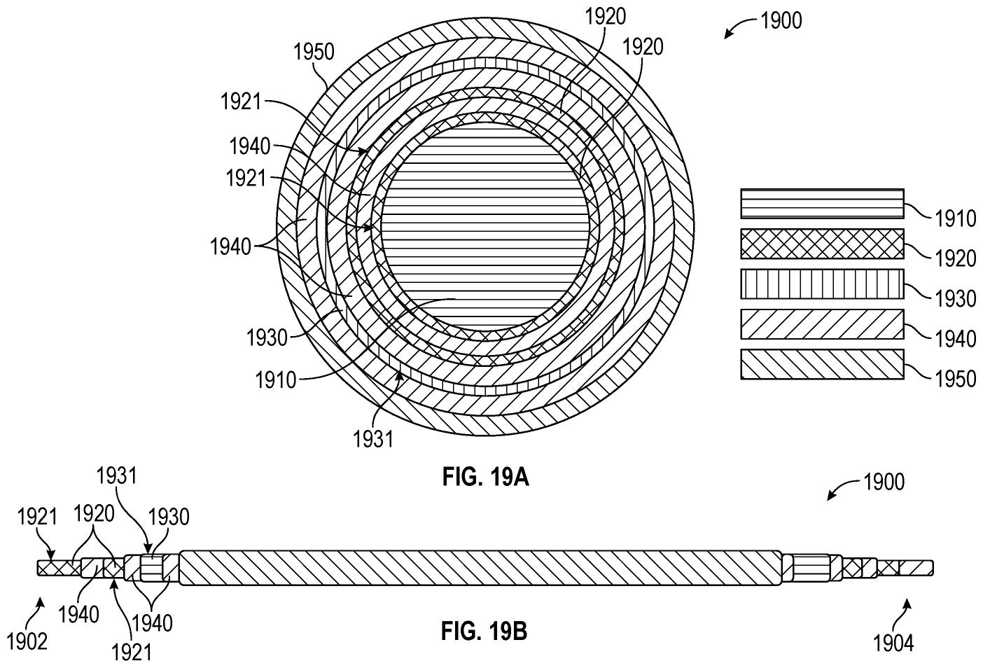

FIGS. 19A-19B illustrate a coaxial analyte sensor assembly 1900 including a polymer core, in accordance with an example. The sensor assembly 1900 can include a polymer core 1910, conductive layers 1920, 1930, insulating dielectric layers 1940, and conductive layer 1950. The conductive layers 1920, 1930, can respectively have sensing areas 1921, 1931, for electrical connection and sensing as electrodes. The sensor assembly 1900 can extend from a first end 1902 to a second end 1904. The first end 1902 can be a sensing end, such as for implantation into a patient for sensing an analyte therein. The second end 1904 can be for electrical connection.

In sensor assembly 1900, the polymer core 1910 can be, for example, a flexible polymer core extending along a central axis of the sensor assembly 1900, such as an LCP polymer. Here, the flexible polymer core 1910 can be coated in several conductive layers 1920, 1930, separated by an insulating dielectric layer 1940. The sensor assembly 1900 can be coated externally by the conductive layer 1950, such as a silver/silver chloride coating, forming a reference electrode. The polymer core 1910 can allow for flexibility of the analyte sensor assembly 1900 and overall tissue compliance.

For example, the flexible polymer core 1910 can be coated with a conductive ink suitable for electrochemical sensing, such as a screen-printing platinum ink. The conductor-coated polymer core 1901 can then coated with an insulating dielectric layer 1940, such as a polyurethane. A second conductive layer 1930 can be coated on top of the insulating dielectric layer 1940, followed by another layer of insulating dielectric layer 1940. This process can be repeated to build up alternating layers of conductor and insulating dielectric, ending with a final conductive layer 1950.

Conductive layers 1920, 1930, can form electrodes, and desired electrode contacts can be formed by selective ablation of material, such as by laser. The conductive layers 1920, 1930 can be a metal or other conductive material suitable for electrochemical sensing, such as graphitic carbon. The conductive layers 1920, 1930 can have the same or different composition for each layer, e.g., all Pt, Pt and C, Au and C, or combinations thereof. The conductive layer 1950 can be formed from Ag/AgCl. In an example, the sensor assembly 1900 stack can have a core 1910 of about 50 μm diameter, conductive layers of about 5 μm thick, insulating layers of about 25 μm thick, and a reference electrode of about 25 μm thick, resulting in a sensor that is about 280 μm in diameter. In some examples, the core 1910 is formed from one or more polymers.

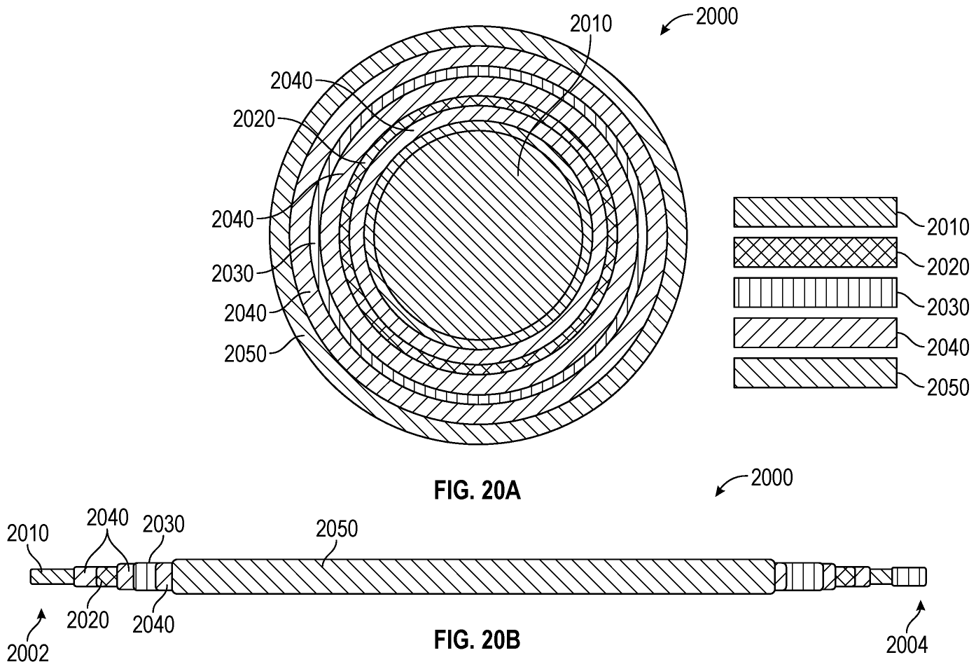

FIGS. 20A-20B illustrate a coaxial analyte sensor assembly 2000 including a wire core, in accordance with an example. The sensor assembly 2000 can include the wire core 2010, which may be formed from a polymer or other material, a first conductive layer 2020, a second conductive layer 2030, insulating layer 2040, and reference electrode layer 2050. The assembly can extend between a first end 2002 and a second end 2004.

In the sensor assembly 2000, the wire core 2010 can extend along the length of the assembly, and the wire core 2010 can be coated in an insulating dielectric layer 2011, such as polyurethane or other insulating material. The wire core 2010 can be made of an electrochemical sensing material. The first conductive layer 2020 can be coated on top of the insulating dielectric layer 2011, followed by another layer of the insulating dielectric layer 2011. Such coatings can be alternated as desired to build up alternating layers of insulation and conductor material. The final electrode can be the most external conductive layer 2050.

Similar to the above sensor assembly 1900, sensing electrodes and electrode contacts can be formed by selective ablation, such as shown in FIG. 20B. The wire core 2010 and conductive layers 2020, 2030 can be a metal or other conductive material suitable for electrochemical sensing, such as graphitic carbon. The wire core and conductive layers can have the same or different composition for each layer, for example, all Pt, Pt and C, Au and C, or combinations thereof. The reference electrode can be formed from Ag/AgCl. In an example, the wire core 2010 can be about 50 μm diameter, the insulating layers 2040 can be about 25 μm thick, and the conductive layers can be about 5 μm thick. In the example analyte sensors assemblies 1900 and 2000, a flexible combination of materials can be used when coating the core component.

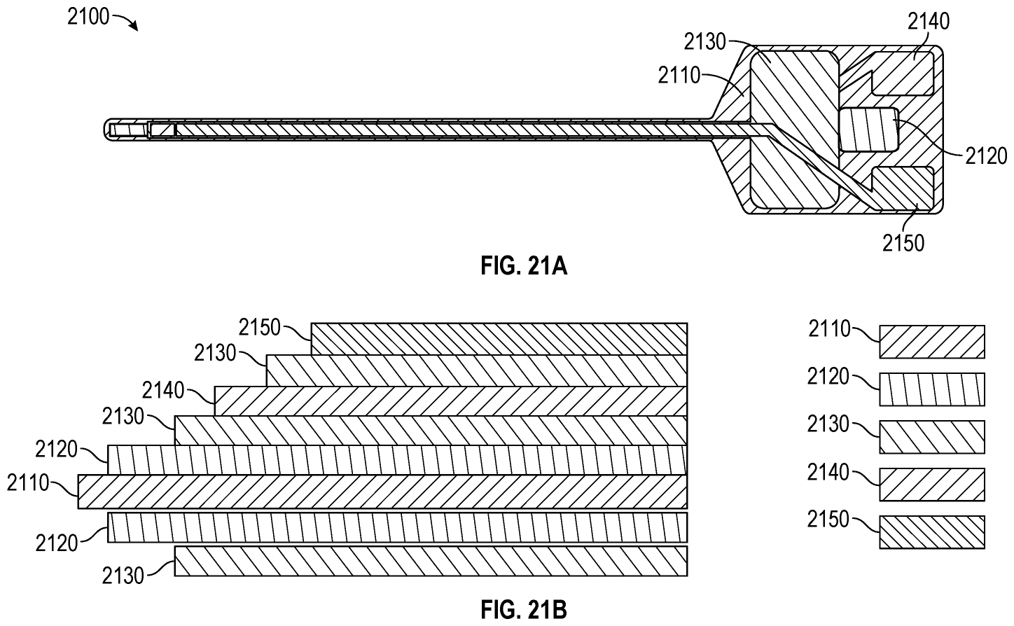

FIGS. 21A-21B illustrate a double-sided stacked planar analyte sensor, in accordance with an example. The sensor assembly 2100 can include a substrate 2110, a first electrode 2120, an insulating layer 2130, a second electrode 2140, and a third electrode 2150. Similar to the sensor assembly 500 discussed above, the sensor assembly 2100 can be stacked such that the sensing end of the assembly 2100 has a staircase-like cross-section.

In the sensor assembly 2100, a three-electrode sensor is shown, with a working electrode (WE) 2120, a counter electrode (CE) 2131 and a reference electrode (RE) 2140. In sensor assembly 2100, the electrodes are stacked or staggered, and geometrically offset from each other along a z-axis. In the sensor assembly 2100, instead of the co-planar arrangement discussed above, multiple conductive layers and insulating layers are stacked. The conductive layers can be staggered for specified exposure of electrodes 2120, 2131, and 2140. In the sensor assembly 2100, the working electrode 2120 can be stacked on both sides of the sensor assembly 2100, such as to allow for two working electrodes.

### Co-Planar Double-Sided Analyte Sensors

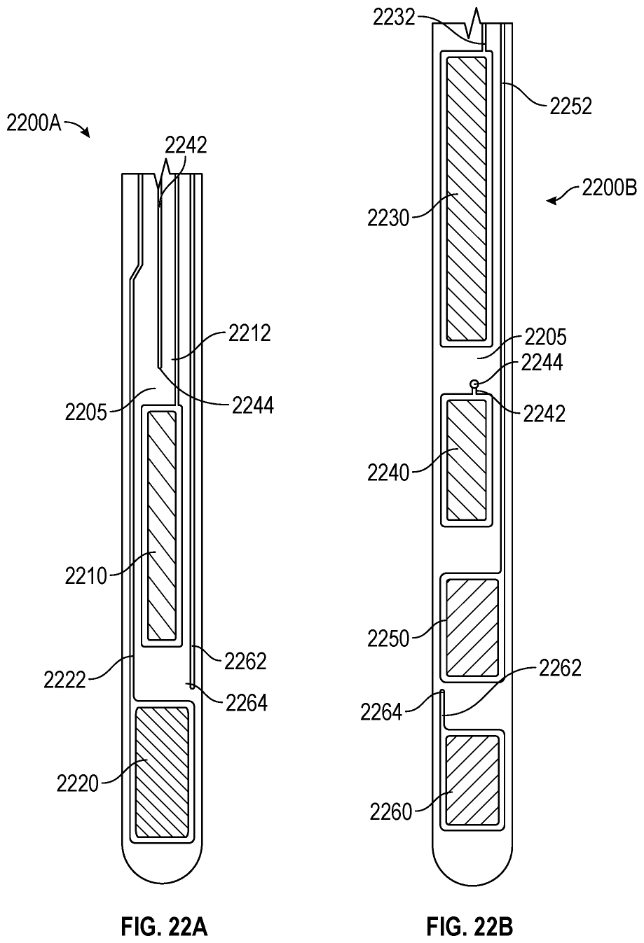

Discussed herein with reference to FIG. 22A to FIG. 28C are several examples of double-sided planar sensors that have multiple electrodes, situated co-planar relative each other. These examples are also often connected through the planar substrate through one or more interconnects. Shown here are the distal portions of the sensors with various electrodes.

For example, a continuous analyte sensor can include a substrate with a first side and a second opposing side. One or more electrodes can be situated on either side of the substrate. The electrodes can include, for example, working electrodes, reference electrode, or counter electrodes, in various amount and configurations. Where two or more electrodes are on a single side of the substrate, those electrodes are substantially co-planar to each other. Each of the electrodes generally has one or more electrical traces extending out from the electrode. The electrical traces can extend to electrical connection pads or points. In some cases, these traces can travel from a first side of the substrate to a second side of the substrate along various interconnects.

For example, FIGS. 22A-22B illustrate a double-sided co-planar analyte sensor 2200. The double-sided co-planar analyte sensor 2200 can have a first side 2200A and a second side 2200B, opposite the first side 2200A. The double-sided co-planar analyte sensor 2200 can include a substrate 2205 with a proximal portion 2202 and a distal portion 2204. Situated on the substrate 2205 on the first side 2200A can be a first reference electrode 2210 with associated reference electrode traces 2212 and a first working electrode 2220 with associated working electrode traces 2222. Situated on the substrate 2205 on the second side 2200B can be a counter electrode 2230 with associated counter electrode traces 2232, a second working electrode 2240 with associated working electrode traces 2242 and interconnects 2244, a second reference electrode 2250 with associated reference electrode traces 2252, and a third working electrode 2260 with associated working electrode traces 2262 and interconnects 2264.

FIG. 22A depicts the first side 2200A of the double-sided co-planar analyte sensor 2200. Here, the first working electrode 2220 and the first reference electrode 2210 are situated co-planar relative each other on the substrate 2205. The first working electrode 2220 is distal of the first reference electrode 2210. In some examples, the placement of the first reference electrode 2210 and the first working electrode 2220 can be switched so that the first reference electrode 2210 is more distal.

FIG. 22B depicts the second side 2200B of the double-sided co-planar analyte sensor 2200. Here, the counter electrode 2230, the second working electrode 2240, the second reference electrode 2250, and the third working electrode 2260 are located on the substrate 2205 co-planar with each other. The third working electrode 2260 can be distal of the other electrodes.

As discussed above, in the presence of one or more analytes, the electrochemical reaction can cause a current to flow between the working electrode(s) and any counter electrode(s), where the raw sensor signal can be based on the current. If present, the reference electrode(s) can provide a stable reference potential and conducts very little current. Any signal from the electrode(s) can be transferred through the traces and interconnects in the double-sided co-planar analyte sensor 2300.

The various electrodes can be co-planar with each other, as opposed to stacked. By positioning the electrodes on the same plane, the overall thickness of the sensor is reduced, as compared to a stacked electrode design. Thinner sensors may improve user comfort during deployment and during wear. Another advantage is that co-planar electrode architecture facilitates case of membrane deposition. The various electrodes can be aligned longitudinally along the substrate 2205. The various electrical traces 2212, 2222, 2232, 2242, 2252, and 2262, associated with each of the corresponding electrodes, can provide electrical connection for the electrodes. For example, the working electrode traces 2262 and 2242 can be run from the more distally located associated working electrodes (2260 and 2240, respectively) down the length of the substrate 2205 towards the proximal portion 2202, such that they can electrically couple the working electrodes 2260 and 2240 to connector pads or points. To avoid crossing these traces with other electrodes on this side of the substrate, the traces can be run through the substrate from the second side 2200B to the first side 2200A as desired.

For example, starting at the third working electrode 2260, the associated working electrode traces 2262 can run proximally from the third working electrode 2260 to an interconnect 2264. At the interconnects 2264, the associated working electrode traces 2262 can run through the substrate from the second side 2200B to the first side 2200A. This can allow for the associated working electrode traces 2262 to continue running proximally along the length of the substrate, towards a connector or connection pad, without running through other electrodes on the second side 2200B, such as the second reference electrode 2250, the second working electrode 2240, or the counter electrode 2230.

Similarly, starting at the second working electrode 2240, the associated working electrode traces 2242 can run from the second working electrode 2240 to an interconnect 2244, through to the first side 2200A of the substrate 2205. This can allow the traces to run proximally from the second working electrode 2240 without running afoul of the counter electrode 2230.

The use of such interconnect to route traces through and along both sides of the planar substrate 2205 can save space on the double-sided co-planar analyte sensor 2200 itself and allow for thinner margins around the electrodes thereon.

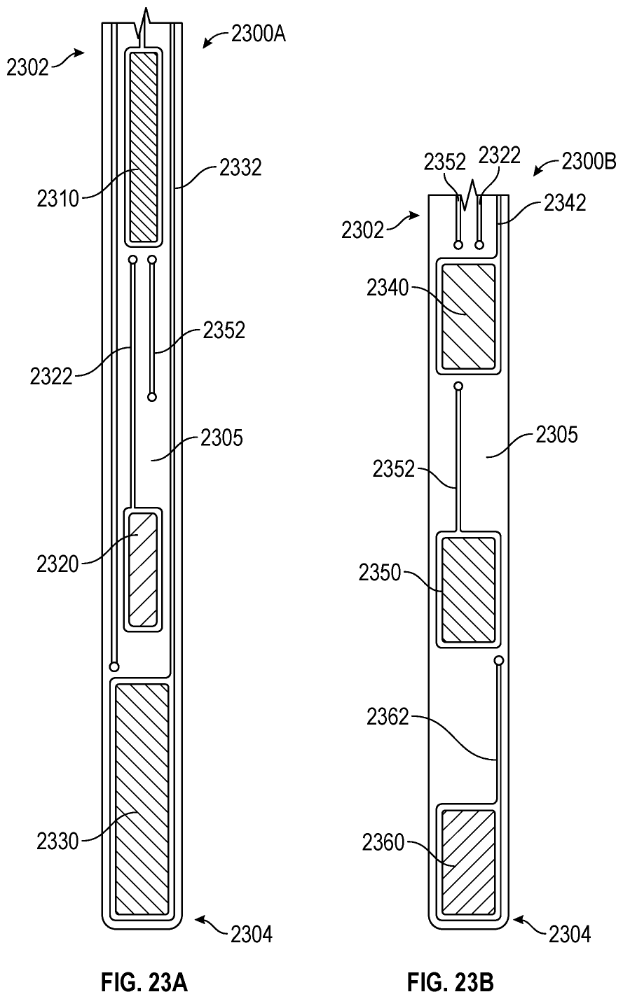

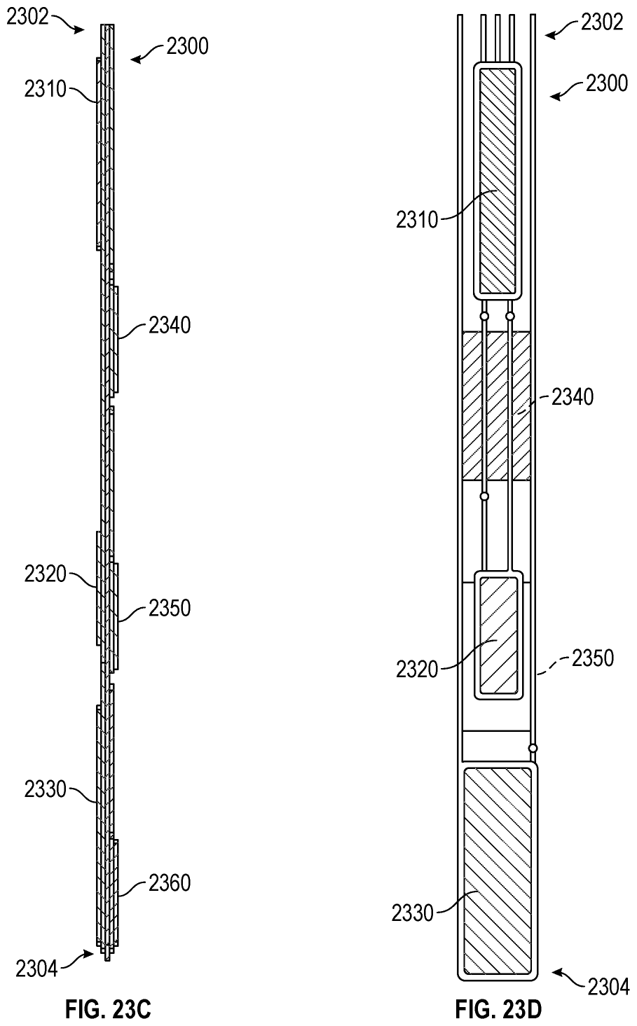

FIGS. 23A-23D illustrate a double-sided co-planar analyte sensor 2300, in accordance with an example. The double-sided co-planar analyte sensor 2300 can have a first side 2300A and a second side 2300B, opposite each other. The double-sided co-planar analyte sensor 2300 can include a substrate 2305 with a proximal portion 2302 and a distal portion 2304.

FIG. 23A depicts a top down view of the first side 2300A. FIG. 23B depicts a top down view of the second side 2300B. FIG. 23C depicts a side view of the double-sided co-planar analyte sensor 2300, showing the electrodes thereon are co-planar with each other. FIG. 23D depicts a view of the double-sided co-planar analyte sensor 2300 without the substrate 2305, so as to visualize the various electrodes, traces, and interconnects.

Situated on the substrate 2305 on the first side 2300A can be a first reference electrode 2310, a second reference electrode 2320 with associated reference electrode traces 2322 and interconnect 2324, and a counter electrode 2330 with associated counter electrode traces 2332. The first reference electrode 2310, the second reference electrode 2320, and the counter electrode 2330 can be situated co-planar relative each other.

The first reference electrode 2310 and the second reference electrode 2320 can be of similar or different sizes. The counter electrode 2330 can be a regular or irregular shape. In an example, the counter electrode 2330 can be distal of the reference electrodes 2320 and 2310.

Situated on the substrate 2305 on the second side 2300B can be a first working electrode 2340 with associated working electrode traces 2342, a second working electrode 2350 with associated working electrode traces 2352 and interconnects 2354, 2356, and a third working electrode 2360 with associated working electrode traces 2362 and interconnects 2364, 2366. Here, all three working electrode 2340, 2350, and 2360, can be on the same side of the substrate 2305, and all co-planar relative each other. The working electrodes 2340, 2350, and 2360 can be roughly the same size, and spaced equally apart from each other.

The electrode traces 2352 associated with the second working electrode 2350 can run proximally along the second side 2300B of the substrate 2305, until the first interconnect 2354, at which point the electrical connection can go through the substrate 2305 to the first side 2300A, where the associated working electrode traces 2352 can continue to run proximally along the substrate 2305. At the second interconnect 2356, the electrical connect can pass back through the substrate 2305 to the second side 2300B, where the associated working electrode traces 2352 can continue to run proximally to allow for electrical connection of the second working electrode 2350 to a connection pad. This can help avoid crossing the associated working electrode traces 2352 with other traces and electrode on the substrate 2305.

Similarly, the working electrode traces 2362 associated with the third working electrode 2360 can be run through the first interconnect 2364 to the first side 2300A, and then back through the interconnects 2366 to the second side 2300B.

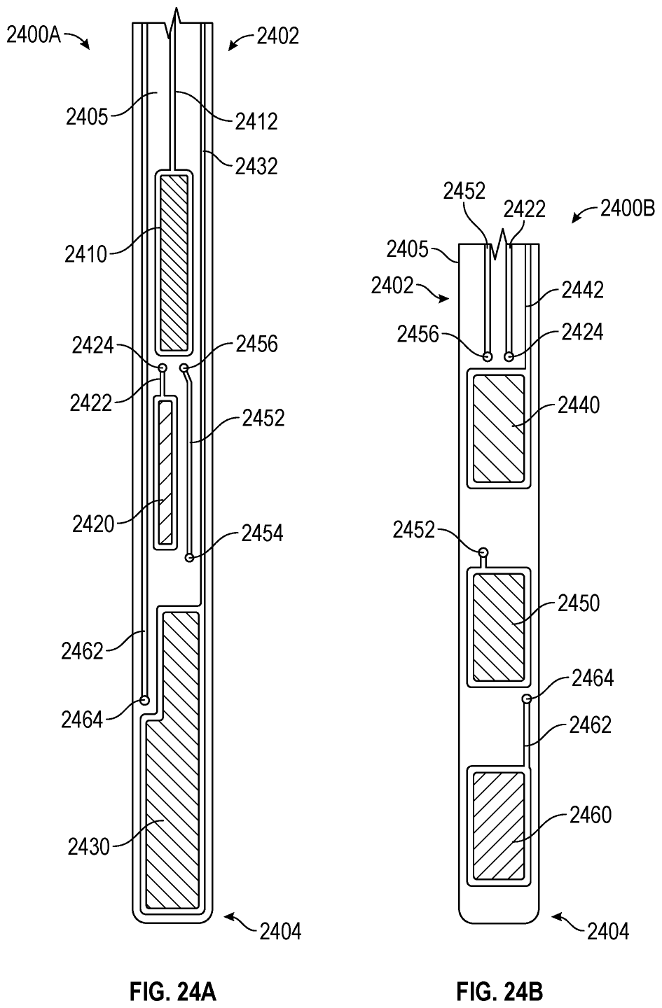

FIGS. 24A-24B illustrate a double-sided co-planar analyte sensor 2400, in accordance with an example. The double-sided co-planar analyte sensor 2400 can have a first side 2400A and a second side 2400B, opposite each other. The double-sided co-planar analyte sensor 2400 can include a substrate 2405 with a proximal portion 2402 and a distal portion 2404.

FIG. 24A depicts a top down view of the substrate 2405 on the first side 2400A. FIG. 24B depicts a top down view of the substrate 2405 on the second side 2400B.

Situated on the substrate 2405 on the first side 2400A can be a first reference electrode 2410 with associated reference electrode traces 2412, a second reference electrode 2420 with associated reference electrode traces 2422 and interconnect 2424, and a counter electrode 2430 with associated counter electrode traces 2432. The counter electrode 2430, the first reference electrode 2410, and the second reference electrode 2420 can be co-planar with each other. The first reference electrode 2410 and the second reference electrode 2420 can be of the same or different sizes. The counter electrode 2430 can be irregularly shaped and can be most distal compared to the reference electrode 2410 and 2420.

Situated on the substrate 2405 on the second side 2400B can be a first working electrode 2440 with associated working electrode traces 2442, a second working electrode 2450 with associated working electrode traces 2452 and interconnects 2454, 2456, and a third working electrode 2460 with associated working electrode traces 2462 and interconnect 2464.

Similar to earlier examples, the traces associated with various electrodes can be routed between the two sides of the substrate through the use of interconnects. This can aid on avoiding crossing traces over other, co-planar electrodes on either side of the substrate.

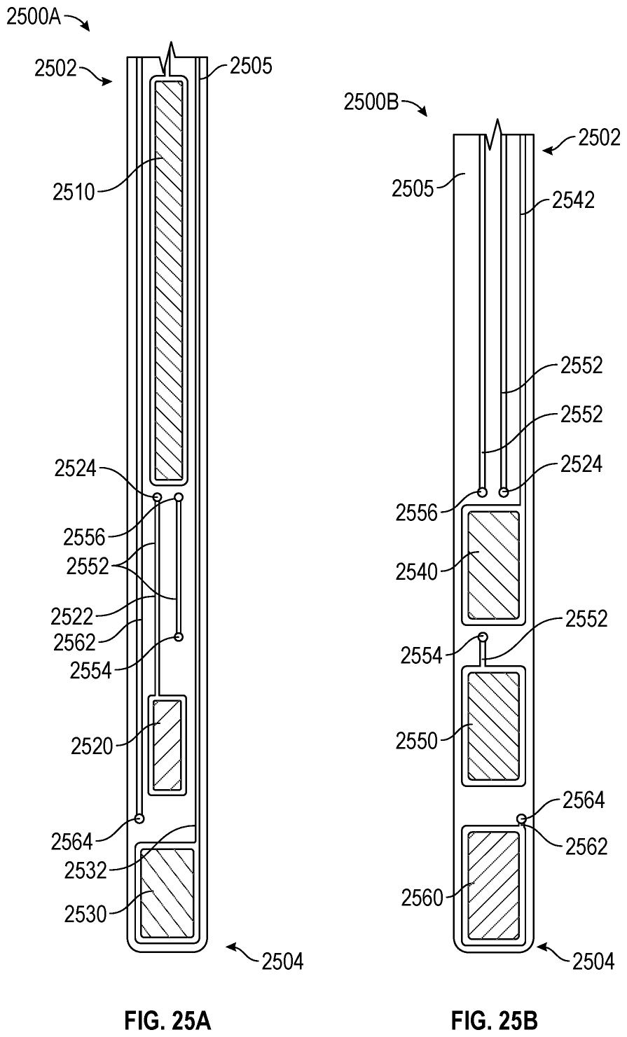

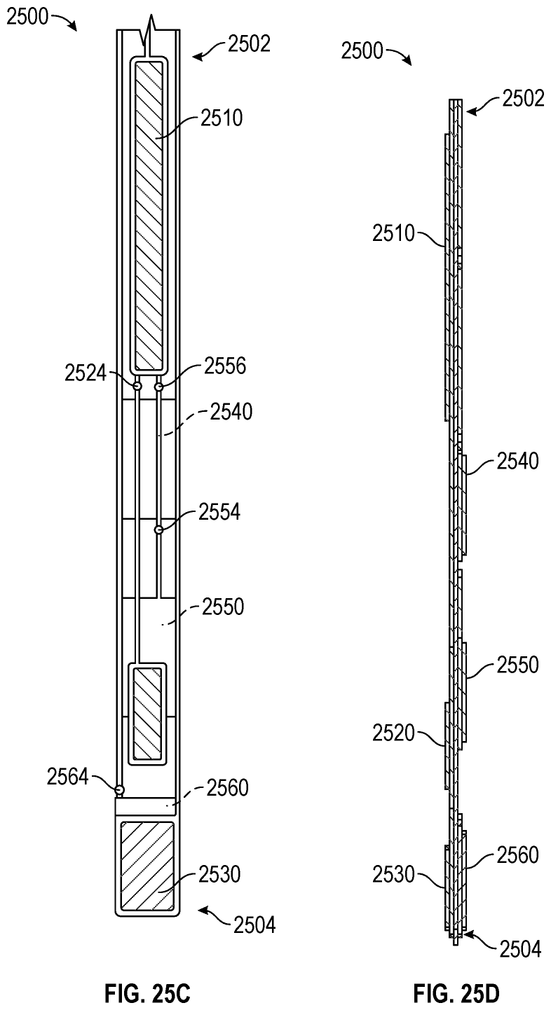

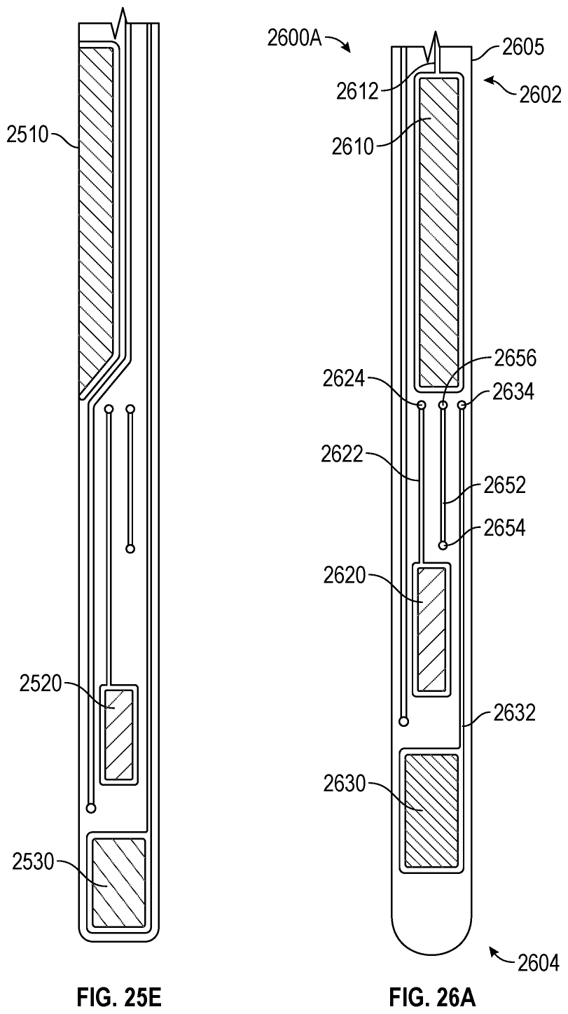

FIGS. 25A-25E illustrate a double-sided co-planar analyte sensor 2500, in accordance with an example. The double-sided co-planar analyte sensor 2500 can have a first side 2500A and a second side 2500B, opposite each other. The double-sided co-planar analyte sensor 2500 can include a substrate 2505 with a proximal portion 2502 and a distal portion 2504.

FIG. 25A depicts a top down view of the first side 2500A of the substrate 2505, while FIG. 25B depicts a top down view of the second side 2500B of the substrate 2505. FIG. 25C depicts a view of the double-sided co-planar analyte sensor 2500 without the substrate 2505, so as to see the various electrodes, traces, and interconnects. FIG. 25D depicts a side view of the double-sided co-planar analyte sensor 2500 including the substrate 2505.

Situated on the substrate 2505 on the first side 2500A can be a counter electrode 2510, a first reference electrode 2520 with associated reference electrode traces 2522 and interconnects 2524, and a second reference electrode 2530 with associated reference electrode traces 2532.

The counter electrode 2510, the first reference electrode 2520, and the second reference electrode 2530 can be co-planar with each other. The first reference electrode 2520 and the second reference electrode 2530 can be of the same or different sizes. The counter electrode 2510 can be relatively rectangular or can have a tapered edges or alternative shape as desired. FIG. 25E depicts a version of the double-sided co-planar analyte sensor 2500 with a counter electrode 2510 having a different shape.

Situated on the substrate 2505 on the second side 2500B can be a first working electrode 2540 with associated working electrode traces 2542, a second working electrode 2550 with associated working electrode traces 2552 and interconnects 2554, 2556, and a third working electrode 2560 with associated working electrode traces 2562 and interconnects 2564.

Similar to earlier examples, the traces associated with various electrodes can be routed between the two sides of the substrate through the use of interconnects. This can aid in avoiding crossing traces over other, co-planar electrodes on either side of the substrate.

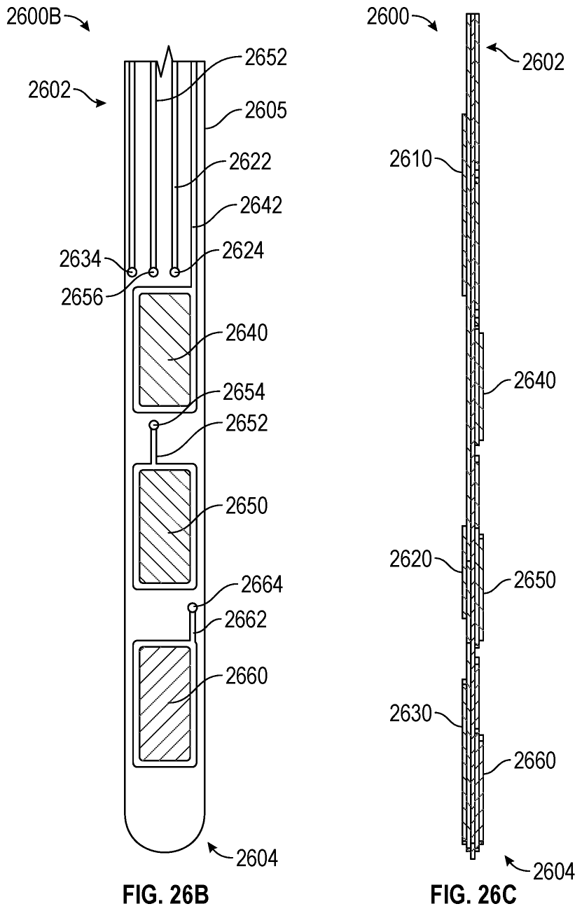

FIGS. 26A-26C illustrate a double-sided co-planar analyte sensor, in accordance with an example. The double-sided co-planar analyte sensor 2600 can have a first side 2600A and a second side 2600B, opposite each other. The double-sided co-planar analyte sensor 2600 can include a substrate 2605 with a proximal portion 2602 and a distal portion 2604.

FIG. 26A depicts a top down view of the double-sided co-planar analyte sensor 2600 on the first side 2600A, while FIG. 26B depicts a top down view of the double-sided co-planar analyte sensor 2600 on the second side 2600B. FIG. 26C depicts a side view of the double-sided co-planar analyte sensor 2600.

Situated on the substrate 2605 on the first side 2600A can be a counter electrode 2610 with associated counter electrode traces 2612, a first reference electrode 2620 with associated reference electrode traces 2622 and interconnects 2624, and a second reference electrode 2630 with associated reference electrode traces 2632 and interconnects 2634.

The counter electrode 2610, the first reference electrode 2620, and the second reference electrode 2630 can be co-planar with each other. The first reference electrode 2620 and the second reference electrode 2630 can be of the same or different sizes.

Situated on the substrate 2605 on the second side 2500B can be a first working electrode 2640 associated working electrode traces 2642 and interconnect 2644, a second working electrode 2650 with associated working electrode traces 2652 and interconnects 2654, 2656, and a third working electrode 2660 with associated working electrode traces 2662 and interconnect 2664. In the double-sided co-planar analyte sensor 2600 the working electrodes 2640, 2650, and 2660, can have equal but reduced spacing therebetween.

Similar to earlier examples, the traces associated with various electrodes can be routed between the two sides of the substrate through the use of interconnects. This can aid in avoiding crossing traces over other, co-planar electrodes on either side of the substrate.

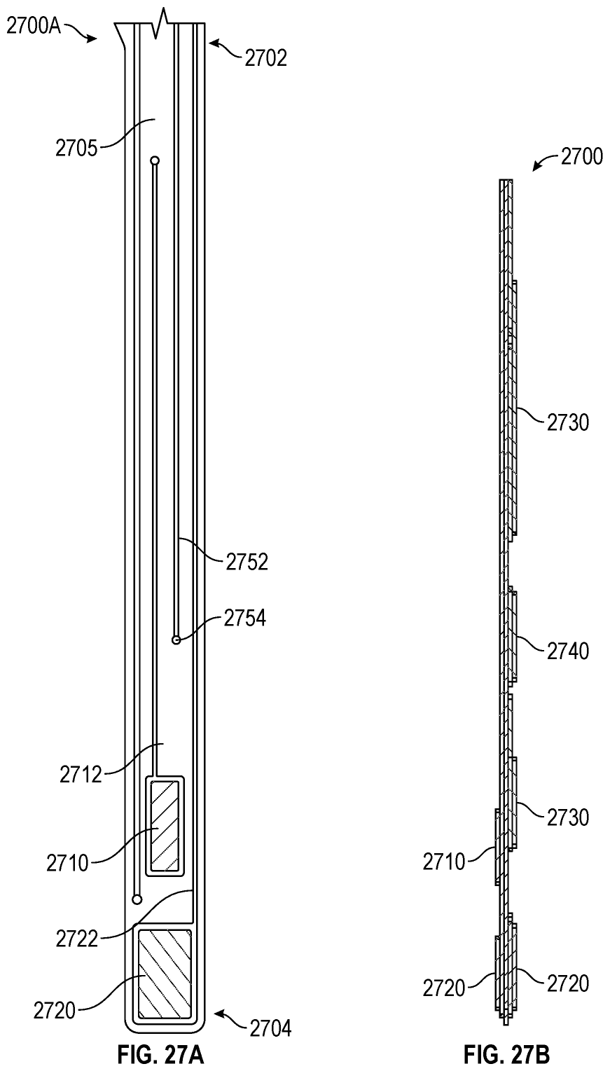

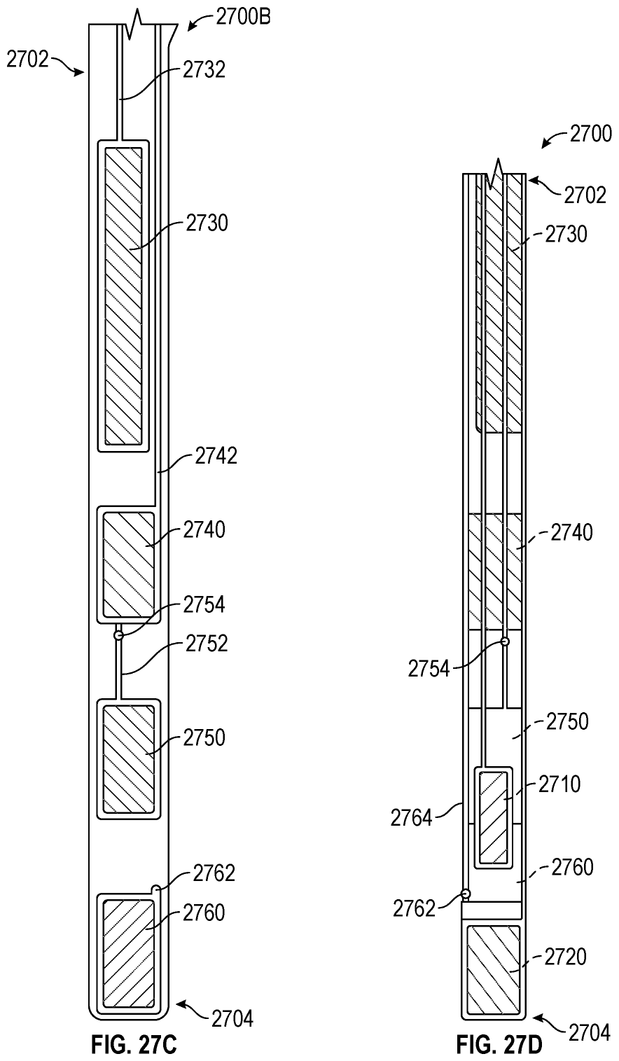

FIGS. 27A-27D illustrate a double-sided co-planar analyte sensor 2700, in accordance with an example. The double-sided co-planar analyte sensor 2700 can have a first side 2700A and a second side 2700B, opposite each other. The double-sided co-planar analyte sensor 2700 can include a substrate 2705 with a proximal portion 2702 and a distal portion 2704.

FIG. 27A depicts a top down view of the double-sided co-planar analyte sensor 2700 on the first side 2700A of the substrate 2705. FIG. 27C depicts a top down view of the double-sided co-planar analyte sensor 2700 on the second side 2700B of the substrate 2705. FIG. 27B depicts a side view of the double-sided co-planar analyte sensor 2700. FIG. 27D shows a view of the double-sided co-planar analyte sensor 2700 with the substrate 2705 so as to see the various electrodes, traces, and interconnects.

Situated on the substrate 2705 on the first side 2700A can be a first reference electrode 2710 with associated reference electrode traces 2712 and a second reference electrode 2720 with associated reference electrode traces 2722. The first reference electrode 2710 and the second reference electrode 2720 can be co-planar with each other. The first reference electrode 2710 and the second reference electrode 2720 can be of similar or differing sizes.

Situated on the substrate 2705 on the second side 2700B can be a counter electrode 2730 with associated counter electrode traces 2732, a first working electrode 2740 with associated working electrode traces 2742, a second working electrode 2750 with associated working electrode traces 2752 and interconnect 2754, and a third working electrode 2760 with associated working electrode traces 2762 and interconnect 2764.

Similar to earlier examples, the traces associated with various electrode can be routed between the two sides of the substrate through the use of interconnects. This can aid on avoiding crossing traces over other, co-planar electrodes on either side of the substrate.

In the double-sided co-planar analyte sensor 2700, having the counter electrode 2730 on the second side 2700B of the substrate 2705, with the working electrodes 2740, 2750, 2760, can allow for more room to increase the area of one or more of the reference electrodes 2710, 2720.

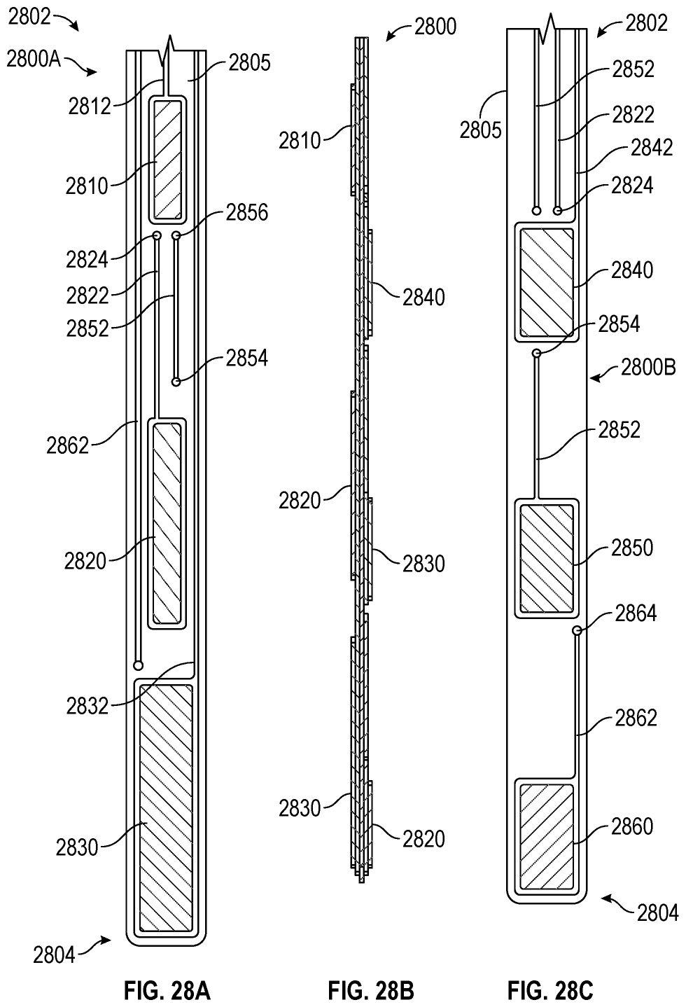

FIGS. 28A-28C illustrate a double-sided co-planar analyte sensor 2800, in accordance with an example. The double-sided co-planar analyte sensor 2800 can have a first side 2800A and a second side 2800B, opposite each other. The double-sided co-planar analyte sensor 2800 can include a substrate 2805 with a proximal portion 2802 and a distal portion 2804.

FIG. 28A depicts a top down view of the double-sided co-planar analyte sensor 2800 on the first side 2800A of the substrate 2805. FIG. 28C depicts a top down view of the double-sided co-planar analyte sensor 2800 on the second side 2800B of the substrate 2805. FIG. 28B depicts a side view of the double-sided co-planar analyte sensor 2700.

Situated on the substrate 2805 on the first side 2800A can be a first reference electrode 2810 with associated reference electrode traces 2812, a second reference electrode 2820 with associated reference electrode traces 2822 and interconnect 2824, and a counter electrode 2830 with associated counter electrode traces 2832. Here, the counter electrode 2830 can be distal of both first reference electrode 2810 and second reference electrode 2820.

Situated on the substrate 2805 on the second side 2800B can be a first working electrode 2840 with associated working electrode traces 2842, a second working electrode 2850 with associated working electrode traces 2852 and interconnects 2854, 2856, and a third working electrode 2860 with associated working electrode traces 2862 and interconnect 2864.

Similar to earlier examples, the traces associated with various electrode can be routed between the two sides of the substrate through the use of interconnects. This can aid on avoiding crossing traces over other, co-planar electrodes on either side of the substrate.

The various configurations of planar sensor electrode layouts depicted and discussed with reference to FIGS. 22A to 28C all include co-planar, double-sided configurations with interconnects through the substrate. These layouts allow for effective positioning of various working electrode(s), counter electrode(s), and reference electrode(s), and their associated traces, while maintaining a smaller overall space.

### Sensor Tip Configurations

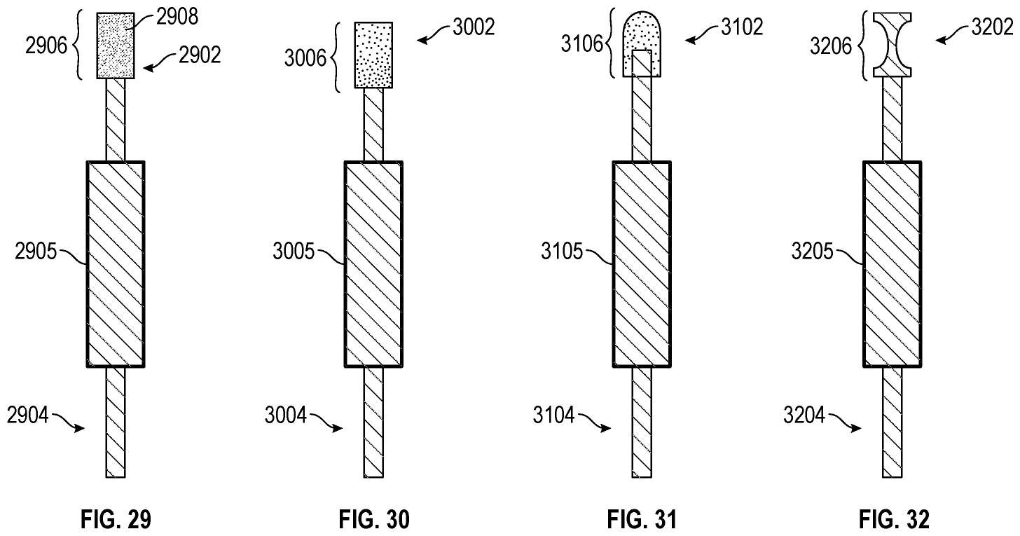

The tip portion of an analyte sensor, such as the planar analyte sensors discussed herein, is often used during insertion of such a transcutaneous sensor. In many cases, an inflammatory response can occur after such an insertion. Shown and discussed with reference to FIGS. 29 to 34 are examples of sensor tip designs to reduce inflammatory response after insertions. For example, many of these designs allow for a reduced amount of tissue injury during insertion.

FIG. 29 illustrates a sensor 2900 with a tip portion 2906, in accordance with an example. The sensor 2900 can have a substrate 2905 that extends between a distal portion 2902 and a proximal portion 2904. The sensor 2900 can include the tip portion 2906 with perforations 2908. The perforations 2908 can allow for the tip portion 2906 to be more flexible or pliable during insertion, such as to reduce tissue damage and inflammation response.

FIG. 30 illustrates a sensor 3000 with a tip portion 3006, in accordance with an example. The sensor 3000 can have a substrate 3005 that extends between a distal portion 3002 and a proximal portion 3004. The sensor 3000 can include the tip portion 3006 made of a soft material. The tip portion 3006 can be made of a material having a softer quality than the material from which the rest of the sensor 3000 is made. For example, the tip portion 3006 can be made of a hydrogel, or elastomer such as silicone, for example. In an example, the tip portion 3006 can be made of a material having a durometer measurement less than that of the substrate 3005 material.

FIG. 31 illustrates a sensor 3100 with a tip portion 3106, in accordance with an example. The sensor 3100 can have a substrate 3105 that extends between a distal portion 3102 and a proximal portion 3104. The sensor 3100 can include the tip portion 3106 that has a cap secured to the distal portion 3102 of the substrate 3105. The tip portion 3106 can, for example, be rounded, and at least partially cover a distal end of the substrate 3105. The cap can be made of a hydrogel, or elastomer such as silicone, for example. In an example, the tip portion 3106 can be a cap that is formed by dipping the distal portion 3102 into such a material.

FIG. 32 illustrates a sensor 3200 with a tip portion 3206, in accordance with an example. The sensor 3200 can have a substrate 3205 that extends between a distal portion 3202 and a proximal portion 3204. The sensor 3200 can include the tip portion 3206 that is bendable such as to provide dampening to piercing force during insertion. The tip portion 3206 can be bendable either by virtue of the material it is made of, or by virtual of its shape, such as by having tapered or indented portions that allow for a more flexible tip portion 3206 relative to the rest of the substrate 3205.

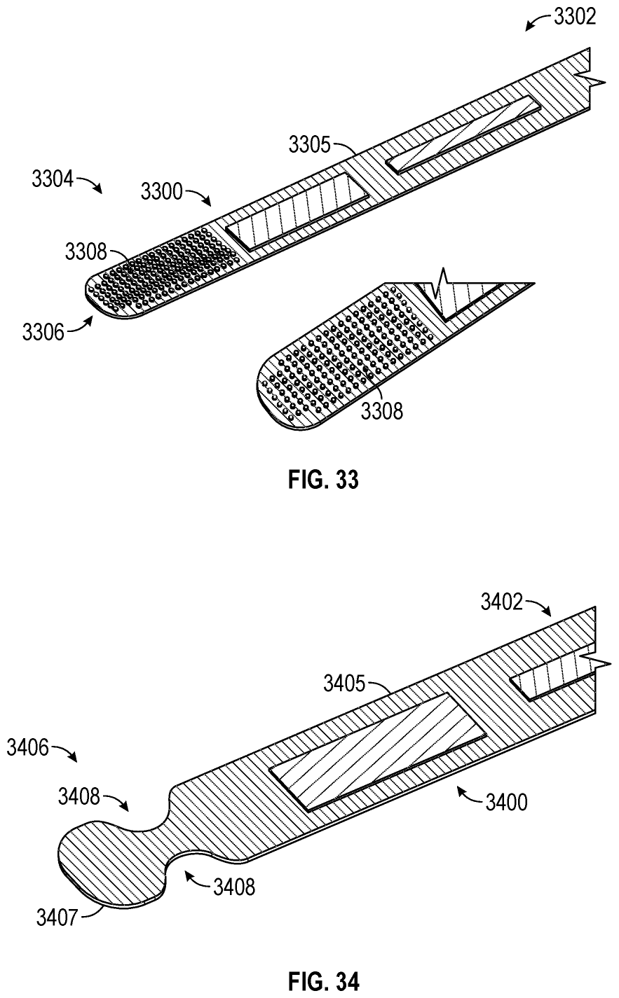

FIG. 33 illustrates a sensor 3300 with a tip portion 3306, in accordance with an example. The sensor 3300 can have a substrate 3305 that extends between a distal portion 3302 and a proximal portion 3304. The sensor 3300 can include the tip portion 3306, which is shown in a zoomed-in view. In this example, the tip portion 3306 can be a monolithic piece with the substrate 3305. The tip portion 3306 can be perforated 3308, such as in a pattern of circles, ovals, or squares. The perforations can be at a regular density along the tip portion 3306 to impart more flexibility thereon.

FIG. 34 illustrates a sensor 3400 with a tip portion 3406, in accordance with an example. The sensor 3400 can have a substrate 3405 that extends between a distal portion 3402 and a proximal portion 3404. The sensor 3400 can include the tip portion 3406. In this example, the tip portion 3406 can be a monolithic piece with the substrate 3405. The tip portion 3406 can include a rounded portion 3407 and two indents 3408. The two indents 3408 can allow for the rounded portion 3407 to move back and forth during insertion and allow for flexibility.

### Amperometric and Potentiometric Combination Sensors

In some cases, more than one sensor type can be included on a planar sensor. In other words, multiple types of sensors can be integrated into the same planar substrate. For example, a planar substrate can host a first sensor system and a second sensor system, both integrated onto a planar substrate. In some cases, the first sensor system and the second sensor system can be situated on the same side of the planar substrate. In some cases, the first sensor system and the second sensor system can be situated on opposing sides of the planar substrate. In some cases, one or both of the first sensor system and the second sensor system can extend across the substrate and have components on both sides of the substrate.

One of the first sensor system or the second sensor system can be, for example, a continuous analyte sensor such as those discussed with reference to FIGS. 22A to 28C above. Such a sensor system can, for example, include a working electrode, a reference electrode, and at least one analyte-sensing membrane.

The first sensor system can be a sensor system configured to take a first type of measurement. The second sensor system can be a sensor system configured to take a second type of measurement, different than the first type of measurement.

For example, one of the sensor systems can be an amperometric sensor, such as those discussed above. Amperometric sensors cause the oxidation or reduction of an electroactive species through the use of a potential applied between a reference and a working electrode. With an amperometric sensor, the resulting current can be measured. Such an amperometric sensor can estimate interstitial glucose values by measuring an electrical current generated by the reaction of glucose either with oxygen or with an immobilized redox mediator. Examples of amperometric sensors can be found in U.S. Application Ser. No. 63/403,568, filed Sep. 2, 2022, entitled “CONTINUOUS MULTI-ANALYTE SENSOR DEVICES AND METHODS,” which is hereby incorporated by reference in its entirety.

In an example, the other sensor can be a different type electrochemical of sensor, such as a potentiometric sensor or a conductometric sensor. For example, the other sensor system can be a potentiometric sensor. In such a sensor system, an electrode or membrane potential can then be measured. For example, sensor measurements can be obtained based on the potential difference between two electrodes. By comparison, with a conductometric sensor, a measurement of conductivity at a series of frequencies can be taken. Examples of potentiometric sensors can be found in U.S. Application Ser. No. 63/403,582, filed Sep. 2, 2022, entitled “DEVICES AND METHODS FOR MEASURING AN ELECTROPHYSIOLOGICAL SIGNAL AND/OR A CONCENTRATION OF A TARGET ANALYTE IN A BIOLOGICAL FLUID IN VIVO”, which is hereby incorporated by reference in its entirety.

In an example potentiometric sensor system, the sensor system can include a first electrode disposed on the substrate, an ionophore disposed on the substrate and configured to selectively transport a target ion to or within the first electrode, and a second electrode disposed on the substrate. In this case, the potentiometric sensor system can further include sensor electronics. Such sensor electronics can be configured to generate a first signal corresponding to a current response, the current response being at least partially based on a reaction at the working electrode corresponding to a concentration of a first analyte, and generate a second signal corresponding to an electromotive force, the electromotive force being at least partially based on a potential difference that is generated between the first electrode and the second electrode responsive to the ionophore transporting the target ion corresponding to a concentration of a second analyte to the first electrode.

In some cases, the second sensor system can be a non-electrochemical sensor. For example, a different type of sensor all together can be integrated onto the substrate, such as an accelerometer or a blood oxygen level sensor. In some cases, the first sensor system and the second sensor system can be directed to different analytes, in addition to different types of measurements.

In some cases, a third sensor system can be integrated into the planar substrate. In this case, the third sensor system may be a different type of sensor, or may be the same type of sensor as either the first or second sensor system.

Sensors with L-Shaped Architectures

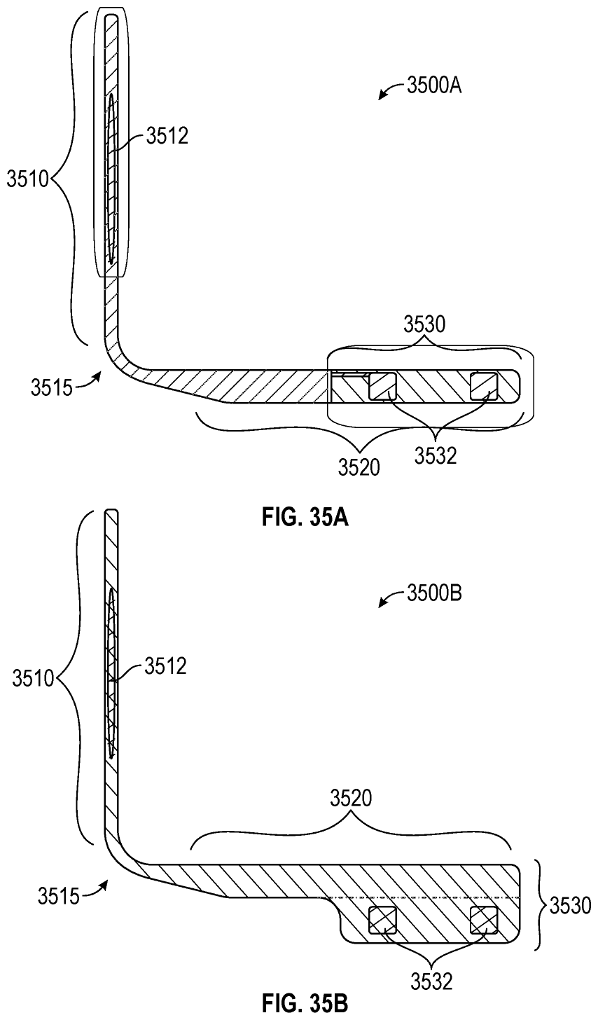

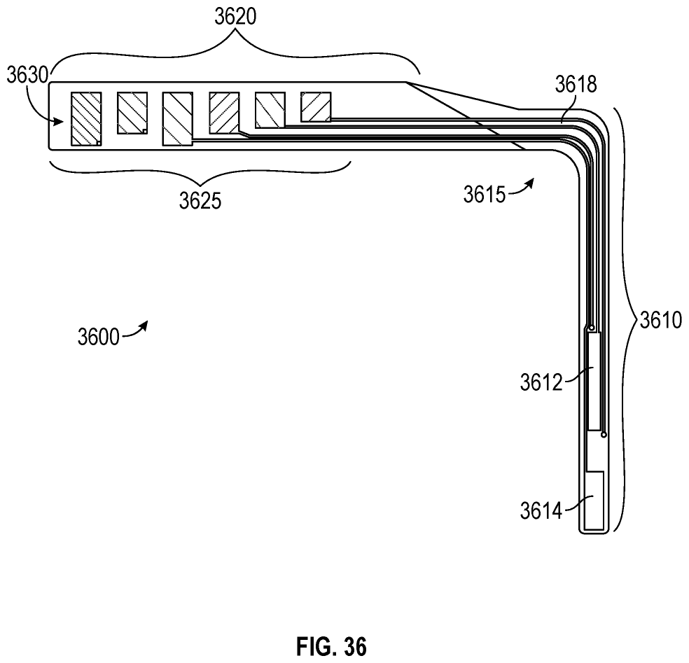

FIG. 35A to 36 depict an example sensor architecture having an overall L-shape, where the substrate itself contains a bend or angle connecting a distal portion and a proximal portion. The L-shaped architecture can be beneficial for insertion and for detectability. In this manner, the L-shaped architecture reduces or eliminates any bias force due to bending an otherwise straight sensor.

FIGS. 35A-35B illustrate an L-shaped sensor 3500, in accordance with an example. FIG. 35A depicts the L-shaped sensor 3500 in a first configuration 3500A, while FIG. 35B depicts the L-shaped sensor 3500 in a second configuration 3500B.

The L-shaped sensor 3500 can include a planar substrate with a distal portion 3510 and a proximal portion 3520. The distal portion 3510 and the proximal portion 3520 can be joined together by a junction portion 3515. The junction portion 3515 can connect the distal portion 3510 and the proximal portion 3520 at a roughly perpendicular angle. The distal portion 3510 and the proximal portion 3520 can be approximately the same length, or of differing lengths.

The distal portion 3510 can include electrodes 3512. A varying number of functional electrodes, such as working electrodes, reference electrode, and counter electrodes, can be on the distal portion 3510. Example electrode layouts are shown and discussed with reference to FIGS. 22A to 28C above. One or more analyte-sensing membranes can extend over the electrodes 3512 as desired.

The proximal portion 3520 can include a connection region that hosts connection pads 3532. The connection pads 3532 can be electrically connected to the electrodes 3512 through traces running through the junction portion 3515. The traces routed through the junction portion 3515 can do so without a need for bending of the flexible printed circuit. The proximal portion 3520 can have a greater width than the distal portion 3510.

The proximal portion 3520 in first configuration 3500A is shown in a folded configuration. The proximal portion 3520 in second configuration 3500B is shown in an unfolded configuration. Folding along the dotted line of the second configuration 3500B can allow for the hosts connection pads 3532 to be parallel to a transmitter printed circuit board assembly. In some cases, the folded portion 3530 can contain one or more interconnects.

FIG. 36 illustrates an L-shaped sensor 3600, in accordance with an example. The L-shaped sensor 3600 can include a distal portion 3610 and a proximal portion 3620 connected by a junction portion 3615.

The distal portion 3610 can include a first electrode 3612 and a second electrode 3614, which can be, for example, working electrodes, reference electrodes, counter electrodes, or combinations thereof. Additional electrodes may be on the opposing side of the substrate. The electrodes 3612, 3614, can be electrically connected through the traces 3618, which run through the junction portion 3615.

The proximal portion 3620 can include the connection pads 3625, which are electrically coupled to the electrodes 3612, 3614 (and any electrodes on the opposite side of the substrate), through the traces 3618. In some cases, the L-shaped sensor 3600 can include a stiffener 3630 on the proximal portion 3620. The stiffener 3630 can provide mechanical and structural support to the L-shaped sensor 3600. Additionally, one or more functional components can be on the proximal portion 3620, such as supported by the stiffener 3630.

Sensors with Cuboidal Architectures

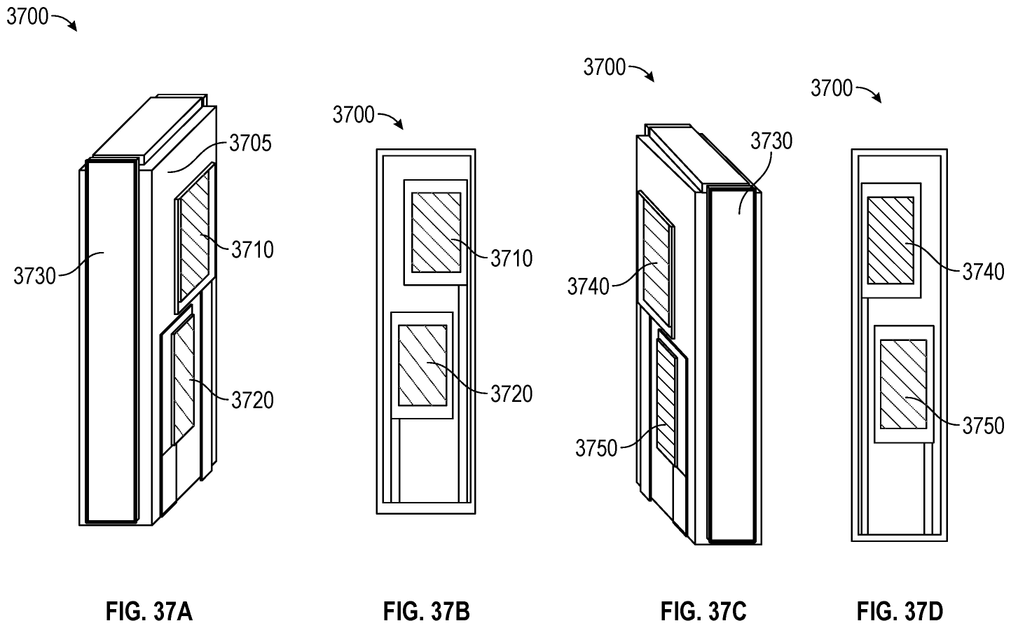

FIG. 37A to 39D depict an example sensor architecture which is cuboidal in nature. Such a sensor architecture may lend itself to multi-analyte sensing and miniaturization of electrodes. Additionally, amperometry sensors can be limited by the space desired for a reference electrode. In the case of a cuboidal sensor, there is additional room on various cuboidal surfaces for such reference electrodes.

FIGS. 37A-37D depict a geometry for multi analyte sensing, in which reference and counter electrodes are on the sides of the cuboidal form factor. This can be done due to small surface areas available for each electrode in planar geometry and the fact that the size of the reference and counter electrode can be small and limiting. In this concept, all or some of the sides of the cuboid can be coated with reference ink and the two larger surfaces can be coated with electrode inks with applied enzyme layers on top of them. This approach can maximize the reactive surfaces. Here, the electrodes can be placed adjacent to each other, either on one plane (front), two planes (front and back) or 3-5 planes (front and back and the sides, in a cuboidal design). The sensors can be PCB based material that have traces and electrodes.

FIGS. 37A-37D illustrate a cuboidal sensor 3700, in accordance with an example. FIGS. 37A and 37B depict a front view of the cuboidal sensor 3700. FIGS. 37C and 37D depict a back view of the cuboidal sensor 3700. The cuboidal sensor 3700 can include a substrate 3705, a first working electrode 3710, a second working electrode 3720, a reference electrode 3730, a third working electrode 3740, and a fourth working electrode 3750.

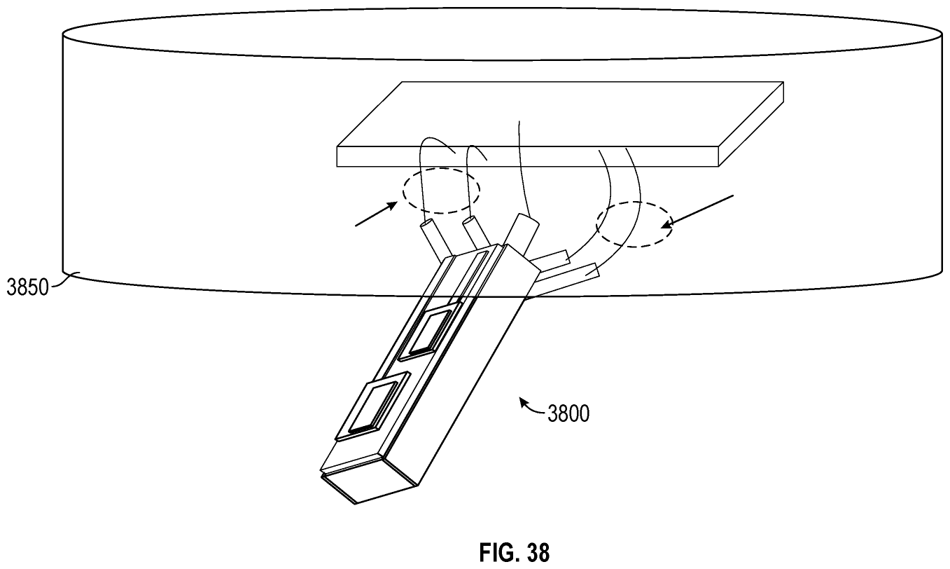

The substrate 3705 can be cuboidal in nature, with six sides. Each of the working electrodes 3710, 3720, 3740, 3750, can be on one of the main faces of the substrate 3705. The reference electrode 3730, by comparison, can be situated on one of the smaller sides of the cuboidal substrate 3705. FIG. 38 illustrates such a cuboidal sensor 3800 operably connected to a circuit board 3850, in accordance with an example.

Each of the working electrodes 3710, 3720, 3740, 3750, can include a membrane thereover. A different type of enzyme layer can be used on each of the working electrodes as desired, such as for detecting multiple types of analytes. In some cases, a string of cuboidal sensors can be used connected to each other.

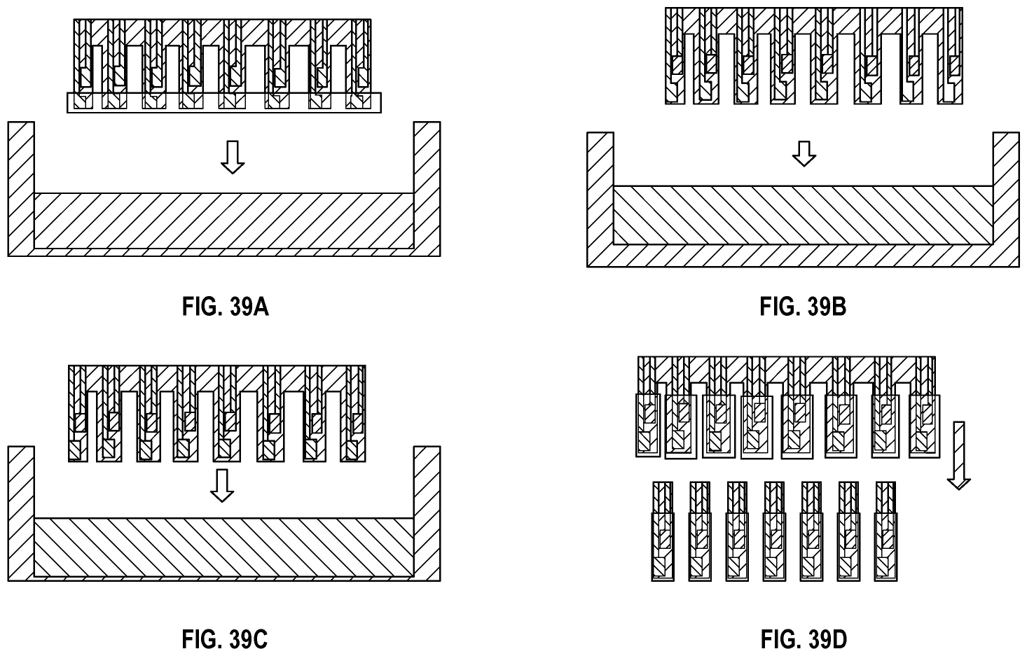

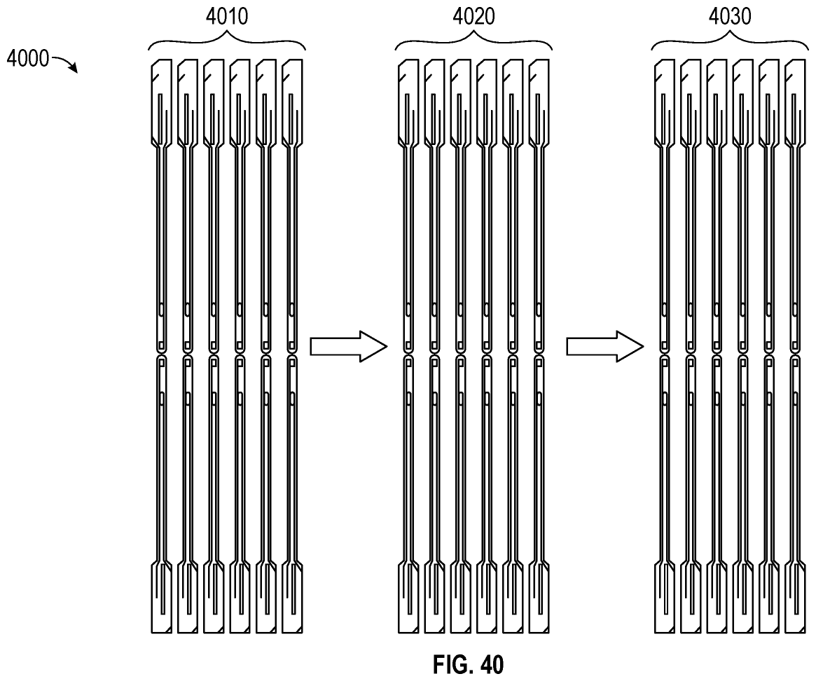

FIGS. 39A-39D depict a method of making a cuboidal sensor, in accordance with an example. When prepared, after application of such enzyme layers on the working electrodes, the cuboidal sensor 3700 can then be coated with varying resistant layers, such as through deposition or dipping. Shown here is an example process of dipping two enzyme layers and then a resistant layer. The process can leverage application of a masking sheet in first step (FIG. 39A) to coat the upper working electrode first, and then dip length parameter control to dip the second enzyme layer as well as the resistant layer (FIG. 39B).

In some cases, more layers of both can be added by changing the dip parameters midway. (FIG. 39C). In some cases, alternative methods of producing the enzyme layers and resistant layers for the working electrodes can be used. For example, they can be spray coated. To facilitate the enzyme layer and resistant layer addition, the sensors can come in an interconnected form factor, as depicted int the figure below.

A variety of approaches could be used to coat reference inks on the sides of the substrate to produce reference electrodes. For example, the larger surfaces can be covered with a masking layer of polymer while the entire sensor is sprayed or deposited with reference ink, the masking layers are then removed for adding membranes and resistant layers for the working electrodes. Reference electrode can also be spray coated to depleted on the sides and tip of the cuboid.

To apply electrical connections to the wearable and transmitter, pins can be connected to each extended electrode. After all the polymer membranes are applied, sensors are then cut to individual sensors, using laser cut or similar methods (FIG. 39D).

### Multi-Analyte Sensors and Methods of Manufacturing Membranes

The sensors discussed herein can include membranes, such as those discussed above, made in a variety of ways. In some cases, the membranes can be targeted to glucose as an analyte. In some cases, the membranes can be targeted to more than one analyte, such as glucose and an additional analyte, like lactate. Where a multi-analyte membrane is used, the membrane may contain more than one type of enzymatic layer to aid in detection of various analytes. In some cases, such a membrane may extend over more than one working electrode. In some cases, different membranes with different enzymatic layers can be produced over different working electrodes, such that individual working electrodes are targeted to specific analytes.

FIG. 40 depicts a flow diagram depicting a method of making planar analyte sensor membranes, in accordance with an example. As discussed above, a variety of methods can be leveraged to produce sensor substrates and electrodes. Likewise, a large variety of methods can be leveraged to produce the membranes used for amperometric (or other electrochemical) sensing of analytes such as glucose or lactate.

For example, a membrane can be deposited onto the electrodes, or formed contemporaneously with the electrodes. In some cases, jet valve dispensing can be used to produce such a membrane. In some cases, slot die coating can be used to produce such a membrane. In some cases, dipping can be used to produce such a membrane. In some cases, discrete dispensing can be used to produce such a membrane. In some cases, enzymatic layers (EZL) are deposited as layers. In some cases, the membrane may include more than on EZL, such as containing one or more analyte sensitive enzymes. In some cases, such a layer can detect multiple analytes, such as glucose, lactate, or other analytes. Such an EZL can often be layered on top of an intermediate layer (IL) that is deposited onto the electrode area. After the IL and the first EZL, additional EZL layers may be deposited. Finally, a resistant layer (RL) may be deposited.

The method 4000 depicted in FIG. 40 depicts a slot die membrane method of producing such membranes. Here, the membranes are produced on a large number of sensor substrate simultaneously, moving from left to right in the figure. First, at step 4010, the aligned sensor substrates are received. Here, the first common IL is deposited through slot-die coating. This common IL is common to all the electrodes being membraned.

Next, at step 4020, EZL layers are produced on top of the common IL. Here in the illustration of FIG. 40, a glucose-sensitive EZL is applied to the central electrodes, while a different EZL (e.g., directed to a different analyte) is deposited on the upper and lower electrodes. After this, at step 4030, the resistant layer is deposited on top of the EZL layers. Again, the middle electrodes are coated with an RL that is directed to glucose, while the top and bottom electrodes are coated with an RL that is directed to a different analyte. In this way, a number of planar sensors are prepared simultaneously, with electrodes targeting multiple analytes.

### Digital Sensors

In various examples described herein, an analyte sensor, such as any of the analyte sensors described herein, is electrically coupled to sensor electronics, such as sensor electronics 106. The electrochemical reaction at the electrodes of the analyte sensor, as described herein, causes the analyte sensor to generate an analog signal indicative of the analyte concentration at the respective electrodes. As described herein, the analog signal may be an electric current in the picoamp range. In some arrangements, the analog signal is provided from the analyte sensor to the sensor electronics via a connector, such as, a connector comprising one or more copper or other metallic contacts.

Passing an analog signal via a connector, however, may lead to certain challenges. For example, the analyte sensor and sensor electronics may be present at or near the skin of a host. In that environment, moisture as well as other contaminants may be present. Moisture, contaminants, and other environmental factors at the host may interfere with the analog signal transmitted across the connector. Also, in some analyte sensors, the analog signal generated by the analyte sensor may have a relatively low magnitude. This may make the analog signal generated by the analyte circuit susceptible to noise, interference, and other signal degradation that may occur at the connector between the analyte sensor the sensor electronics.

These concerns may add cost and/or complexity to the analyte sensor system. For example, it may be desirable to make the connector as robust as possible including, for example, sealing the connector or even sealing the sensor assembly. For example, the connector may be positioned within an enclosure with sufficient structural strength to avoid deformation, and may include a gasket or other suitable sealing mechanism to prevent moisture from reaching electrical contacts of the connector. In some examples, the connector may include contacts that are or are mechanically coupled to a spring tending to maintain electrical coupling between the contacts. Even these mitigation measures, however, may not completely address signal degradation at the connector. The noise arising from passing the analog signal via the connector may still degrade the performance of the analyte sensor system and/or lead to premature failure.

Various examples described herein address these and other challenges by utilizing an analyte sensor comprising a substrate having an analog front end circuit mechanically and electrically coupled to the substrate. The substrate may comprise one or more traces that are electrically coupled to one or more electrodes of the analyte sensor and also electrically coupled to the analog front end circuit. In some examples, the mechanical coupling of the analog front end circuit to the substrate shields connections between the analog front end circuit and the one or more traces, thereby protecting those connections from moisture, contaminants, and other environmental conditions.

The analog front end circuit may comprise various sub-circuits for conditioning the analog signal received from the analyte sensor. In some examples, the analog front end circuit may comprise an analog-to-digital converter to convert the analog signal to a digital signal. The digital signal may be provided to sensor electronics via a connector, such as a connector comprising one or more copper or other metallic contacts. Because the connector is after the analog front end circuit in the signal chain, the signal transmitted across the connector may be digital (generated by the analog-to-digital converter at the analog front end circuit). Because a digital signal may not be as susceptible to noise and other signal degradation as an analog signal, this arrangement may mitigate noise concerns associated with the connector.

In some examples, a calibration check is applied to an analyte sensor, for example, during manufacture. The calibration check may be applied, at least in part, to detect any manufacturing defects in the analyte sensor. If a detected manufacturing defect to the analyte sensor renders the analyte sensor unsuitable for use, the defective sensor may be discarded. In various examples, mechanically coupling the analog front end circuit to the substrate of the analyte sensor may reduce the risk of sensors being discarded during a calibration check. For example, some portion of calibration check failures may be attributable to the degradation of the analog sensor signal between the analyte sensor itself and one or more testing probes. In arrangements, such as those described herein, in which an analog front end circuit is mechanically and electrically coupled to the substrate of the analyte sensor, a calibration check may be performed using one or more digital signals generated by the analog front end circuit. Because a digital signal may not be as susceptible to degradation in the presence of moisture and/or other environmental factors, this may reduce the number of calibration check failures due to signal degradation, thus reducing total number of analyte sensors scrapped during manufacturing.

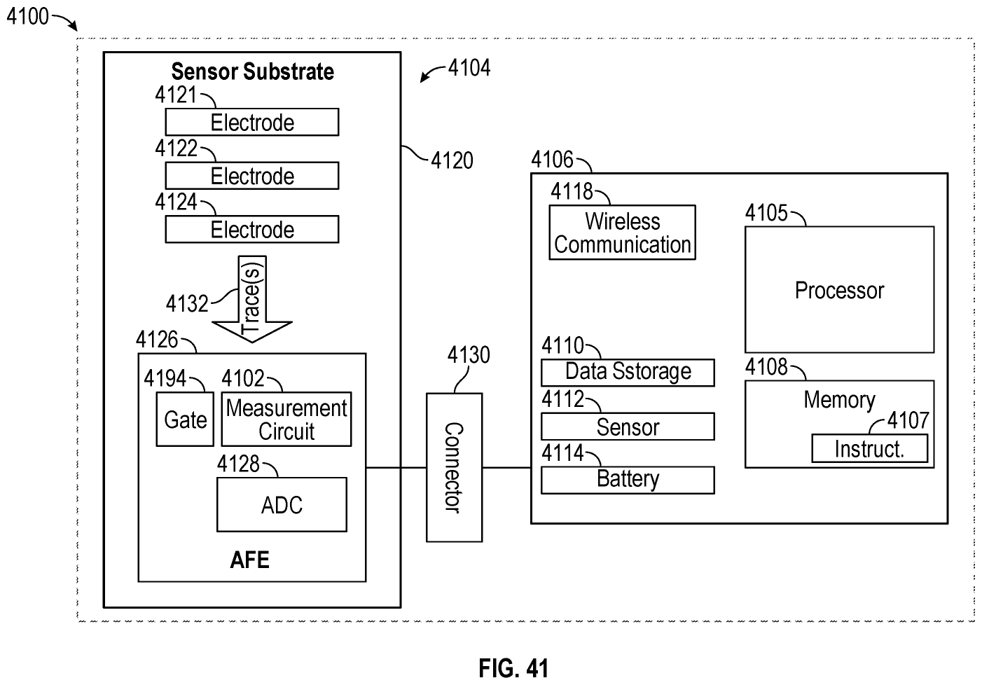

FIG. 41 is a diagram showing one example of an analyte sensor system 4100 including an analyte sensor 4104 electrically coupled to sensor electronics 4106 via a connector 4130. The analyte sensor 4104 may comprise an analog front end circuit 4126. Accordingly, the raw sensor signal passed to the sensor electronics 4106 via the connector 4130 may be a digital signal rather than an analog signal. As described herein, this may reduce signal degradation across the connector 4130 due to moisture and/or other environmental conditions. In some examples, the connector 4130 comprises one or more sensor-side contacts electrically coupled to the analog front end circuit 4126 and one or more electronics-side contacts electrically coupled to the sensor electronics 4106. The one or more sensor-side contacts and one or more electronics-side contacts may be in physical contact with one another to electrically couple the analog front end circuit 4126 to the sensor electronics 4106.

The analyte sensor 4104 may comprise electrodes 4121, 4122, 4124 that generate one or more electrical signals, as described herein. The electrodes 4121, 4122, 4124 may be arranged in any suitable manner. In some examples, the analyte sensor 4104 may be configured according to a three-electrode arrangement comprising a working electrode, a counter electrode, and a reference electrode. In other examples, the analyte sensor 4104 may be arranged according to a two-electrode configuration comprising a working electrode and a reference/counter electrode. Also, it will be appreciated that, in some examples, the analyte sensor 4104 may comprise more than one working electrode. As described herein, different working electrodes may be configured to generate electrical signals indicating the concentration of different analytes.

The analyte sensor 4104 may comprise a sensor substrate 4120. The sensor substrate 4120 may be a planar substrate, for example, as described herein. Any suitable material may be used for the planar substrate. In some examples, the planar substrate may be constructed from a glass-reinforced epoxy resin which may meet suitable standards such as, for example, FR4 standard promulgated by the National Electrical Manufacturers Association (NEMA). In some examples, all or part of the sensor substrate 4120 is a flexible substrate, such as, for example similar to the substrate 410 described herein. Electrodes 4121, 4122, 4124, in some examples, may be bonded to the sensor substrate 4120, for example, as described herein. Sensor substrate 4120 may also comprise conductive traces 4132 deposited thereon. The conductive traces 4132 may electrically couple the various electrodes 4121, 4122, 4124 to the analog front end circuit 4126.

The analog front end circuit 4126 may comprise various signal conditioning circuits such as, for example, a gate circuit 4194 similar to the gate circuit 294, a measurement circuit 4102 similar to the measurement circuit 202, and/or the like. For example, measurement circuit 4102 may be and/or include a potentiostat, similar to the measurement circuit 202 described herein. The analog front end circuit 4126 may also comprise an analog-to-digital converter 4128. The analog-to-digital converter 4128 may receive an analog signal and convert the analog signal to a digital signal. The digital signal generated by the analog-to-digital converter 4128 may be provided to the sensor electronics 4106 via the connector 4130.

Sensor electronics 4106 may include various components, for example, similar to the components of the sensor electronics 106 described in FIG. 2. For example, the sensor electronics 4106 may include a processor 4105 similar to the processor 204; a memory 4108 similar to the memory 208; instructions 4107 similar to the instructions 206; data storage 4110 may be similar to data storage memory 210; one or more sensors 4112 similar to the sensor 212; a battery 4114 similar to the battery 214; a wireless communication circuit 4118 similar to the wireless communication circuit 218; and/or the like. It will be appreciated that, in some examples, the sensor electronics 4106 may include various other components not shown in FIG. 41 and/or may omit some components shown in FIG. 41. In some examples, the sensor electronics 4106 may be in communication with one or more peripheral devices, such as the peripheral device 250, and/or one or more medical devices, such as the medical device 270.

The analog front end circuit 4126 may be arranged and packaged in any suitable manner. In some examples, the analog front end circuit 4126 may comprise an analog front end chip that includes a substrate having various components positioned thereon. For example, the substrate may be or include a silicon wafer with some or all of the components of the analog front end circuit 4126 fabricated on the silicon wafer. The silicon wafer may be packaged in any suitable enclosure. Also, in some examples, the analog front end circuit 4126 may comprise an FR4 or similar substrate with the various components of the analog front end circuit 4126 connected thereon. In some examples, the analog front end circuit 4126 may be or include a commercially-available analog front end circuit chip.

The various inputs and outputs of the analog front end circuit 4126 may comprise conductive contacts arranged as pins, pads, and/or the like. The analog front end circuit 4126 may be mechanically and electrically coupled to the sensor substrate 4120 in any suitable manner. For example, the analog front end circuit 4126 may be surface mounted to the sensor substrate 4120. The surface mount may include making an electrical connection between contacts of the analog front end circuit 4126 and corresponding contacts formed on the sensor substrate 4120. The electrical connections may be made using any suitable technique including, for example, aluminum wedge bonding, wire bonding, underfill, and/or the like. In some examples, the technique for generating an electrical connection may also mechanically couple the analog front end circuit 4126 to the sensor substrate 4120. Also, in some examples, the analog front end circuit 4126 may be mechanically coupled to the sensor substrate 4120 using a mechanism that is in addition to or instead of mechanical coupling provided by the electrical connection. For example, a bonding agent, such as a non-conductive epoxy, may be used to mechanically couple the analog front end circuit 4126 to the sensor substrate 4120.

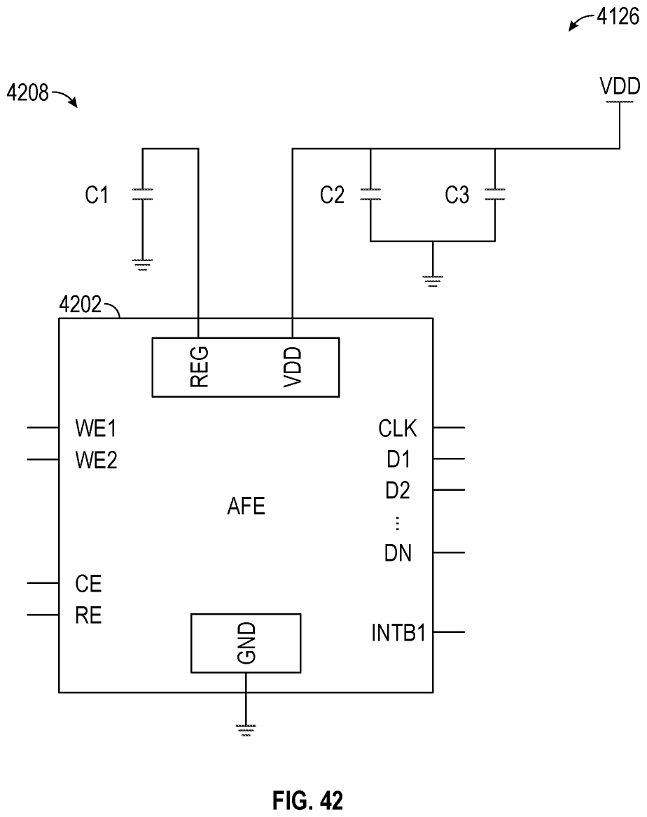

FIG. 42 is a diagram showing one example arrangement of the analog front end circuit 4126. In the example of FIG. 42, the analog front end circuit 4126 comprises an analog front end chip 4202 and additional analog front end components 4208. The analog front end chip 4202 comprises various inputs and outputs. For example, the inputs and outputs of the analog front end chip 4202 may be implemented as pins, surface mount pads, and/or any other suitable connector arrangement. Example inputs to the analog front end chip 4202 include an input voltage (VDD), a regulator input (REG), and ground (GND).

In the example of FIG. 42, the analog front end chip 4202 comprises various inputs and outputs for interfacing with the sensor electronics 4106. In some examples, inputs and outputs for interfacing with the sensor electronics 4106 are digital inputs and outputs. These may include, for example, a clock input (CLK), an interrupt line (INTB1), and various data input/outputs (e.g., D1, D2 . . . , DN). The clock input may receive a clock signal generated by the sensor electronics 4106 (e.g., by and/or for the processor 4105). The interrupt line may be used by the analog front end chip 4202 to trigger one or more interrupts at one or more processors of the sensor electronics 4106, such as the processor 4105. The data input/outputs may be arranged to provide data to the sensor electronics 4106 and/or to receive data from the sensor electronics 4106. In some examples, the data input/outputs may be arranged for bidirectional data transfer and may include, for example, a Master Out Slave In (MOSI) input/output and a Master In Slave Out (MISO) input/output. In some examples, data being transmitted from the analog front end chip 4202 to the sensor electronics 4106 may be provided at the MISO input/output and data being transmitted from the sensor electronics 4106 to the analog front end chip 4202 may be transmitted via the MOSI input/output.

Inputs and outputs of the analog front end chip 4202 that are four communicating with the sensor electronics 4106 may be electrically coupled to the connector 4130 and may pass to and/or from the sensor electronics 4106 via the connector 4130. As described herein, raw sensor signal provided to the sensor electronics 4106 via the connector 4130 by the analog front end circuit 4126 may be a digital signal, thus mitigating signal degradation at the connector 4130.

FIG. 42 also shows inputs and outputs of the analog front end chip 4202 that are for interfacing with the electrodes 4121, 4122, 4124. Inputs and outputs for interfacing with the electrodes 4121, 4122, 4124 may be analog inputs and outputs. That is, for example, signals passed at the inputs and outputs that are for interfacing with the electrodes 4121, 4122, 4124 may be analog signals. In the example of FIG. 42, these include a reference electrode input/output (RE), a counter electrode input/output (CE), a first working electrode input/output (WE1), and a second working electrode input/output (WE2). It will be appreciated that the inputs and outputs of the analog front end circuit 4126 for interfacing with the electrodes 4121, 4122, 4124 may vary, for example, depending on the electrodes that are included in the analyte sensor 4104. Also, in various examples, the analog front end chip 4202 may receive inputs from the various electrodes via the described inputs/outputs. For example, as described herein, electrical currents provided by the working electrode or electrodes may indicate analyte concentration at the analyte sensor 4104. Also, in some examples, the analog front end circuit 4126 may provide bias signals to one or more of the electrodes.

FIG. 42 also shows analog front end components 4208 that are outside of the analog front end chip 4202. These include, for example, a capacitor C1 electrically coupled between ground and the regulator pin of the analog front end chip 4202. Also, capacitors C2 and C3 are electrically coupled as shown between the input voltage VDD and the input voltage pin VDD of the analog front end chip 4202. The capacitors C1, C2, C3 may provide power conditioning on the voltage and ground lines of the analog front end chip 4202. It will be appreciated that, in various examples, there may be various different configurations of power conditioning capacitors and/or other components outside of an analog front end chip such as the analog front end chip 4202. In some examples, power conditioning capacitors may be fabricated on the sensor substrate 4120 utilizing printing or any other suitable technique. Connections between various capacitors and corresponding input/outputs of the analog front end chip 4202 and input voltage VDD may be provided by traces fabricated on the sensor substrate 4120 as described herein.

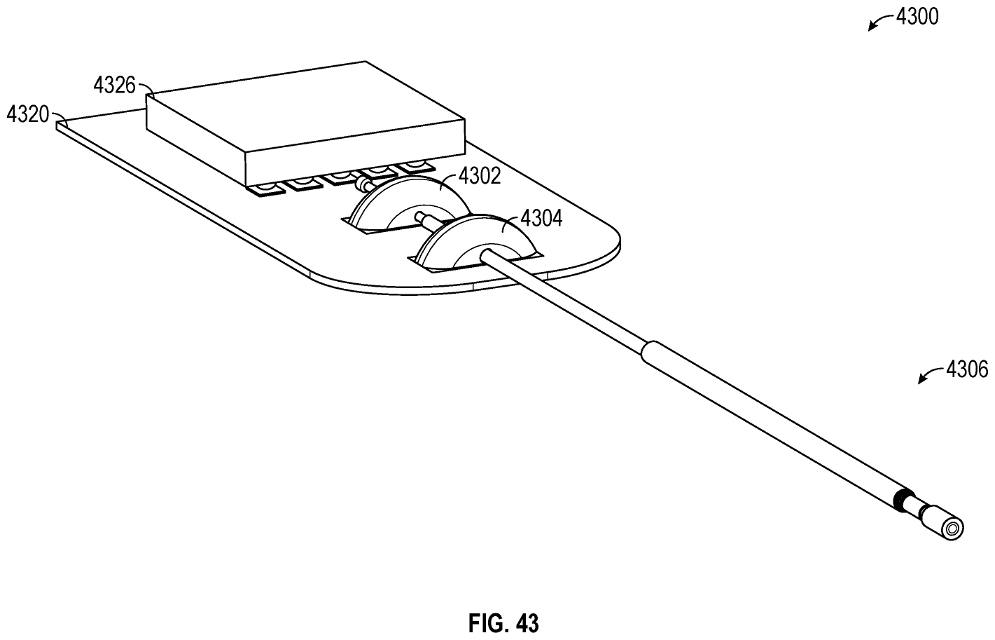

FIG. 43 is a diagram showing one example arrangement of an analyte sensor system 4300 comprising an analog front end circuit 4326. In the example of FIG. 43, the analyte sensor system 4300 comprises an electrode subassembly 4306 arranged in a coaxial manner. For example, the electrodes of the electrode subassembly 4306 may be in any suitable arrangement such as, for example, the arrangements described herein including with respect to FIGS. 3B, 3C, 15A-D, 16A-D, 17A-D, 18, 19A, 19B, 20A, and 20B. Electrodes of the electrode subassembly 4306 may be electrically coupled to a sensor substrate 4320 connectors 4302 and 4304. Connectors 4302 and 4304 may be any suitable connectors including, for example, a stamped metal type connector. Traces (not shown in FIG. 43) may electrically couple the electrodes of the electrode subassembly 4306 to the analog front end circuit 4326, as described herein.

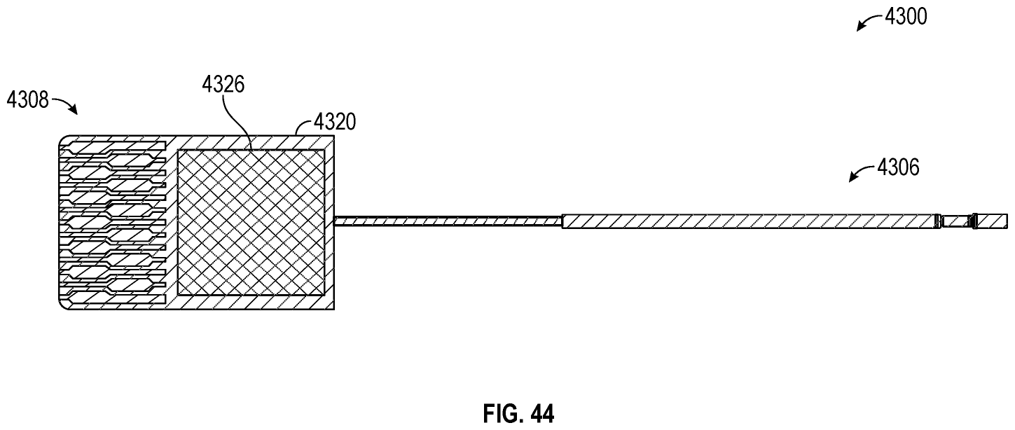

FIG. 44 is a diagram showing another example arrangement of the analyte sensor system 4300. The example of FIG. 44 includes a connector portion 4308. In this example, the connector portion 4308 may represent a portion of a zero insertion force (ZIF) connector. The connector portion 4308 may be coupled with a connector such as the connector 4130 to couple the analog front end circuit 4326 to the sensor electronics 4106. For example, the sensor substrate 4320 may comprise traces electrically coupled to input/outputs of the analog front end circuit 4326 that are to interface with the sensor electronics 4106.

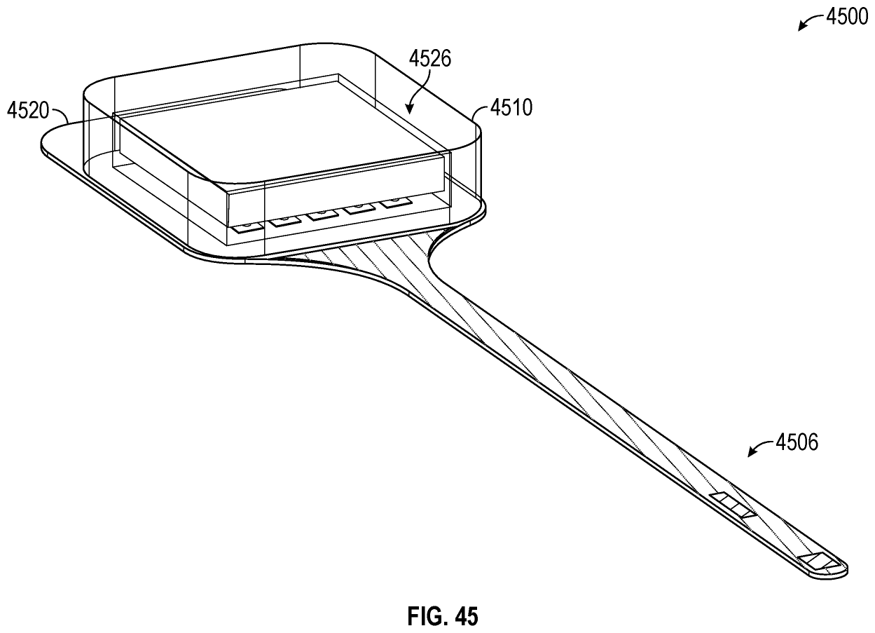

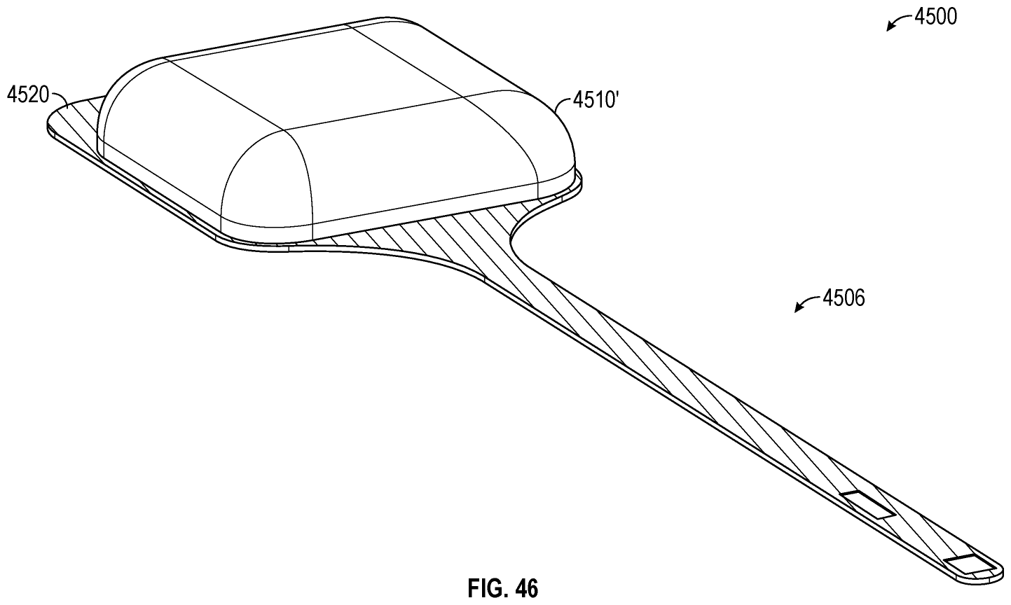

FIG. 45 is a diagram showing another example arrangement of an analyte sensor system 4500 comprising an analog front end circuit 4526. In the example of FIG. 45, the analyte sensor system 4500 comprises an electrode subassembly 4506 having electrodes fabricated on the sensor substrate 4520. For example, the electrodes of the electrode subassembly 4506 may be arranged in any suitable manner including, for example, those described herein with respect to FIGS. 4A-G, 5A-K, 6A-E, 7A-1, 8A-F, 9, 10A-B, 11A-C, 12, 13, 14A-D, 21A, 21B, 22A-B, 23A-D, 24A-B, 25A-E, 26A-C, 27A-D, 28A-C, 29-34, 35A-B, 36, 37A-D, and 38-40. Traces (not shown in FIG. 45) may electrically couple the electrodes of the electrode subassembly 4506 to the analog front end circuit 4526, as described herein.

In the example of FIG. 45, the analog front end circuit 4526 is positioned within an enclosure 4510. The enclosure 4510 is mechanically coupled to the analog front end circuit 4526 and/or the sensor substrate 4520. Any suitable mechanical coupling may be used. In some examples, the enclosure 4510 is snap-fit to the sensor substrate 4420. Also, in some examples, the enclosure 4510 is bonded to the sensor substrate 4520 using a bonding agent, such as, a suitable epoxy. Also, in some examples, the enclosure 4510 may be molded onto the sensor substrate. FIG. 46 is a diagram showing an example of the analyte sensor system 4500 comprising an enclosure 4510′ that is molded onto the sensor substrate 4520. In various examples, as described herein, the enclosure 4510, 4510′ may shield the analog connections between the various electrodes and the analog front end circuit 4426 from moisture and other environmental conditions that may lead to signal degradation.

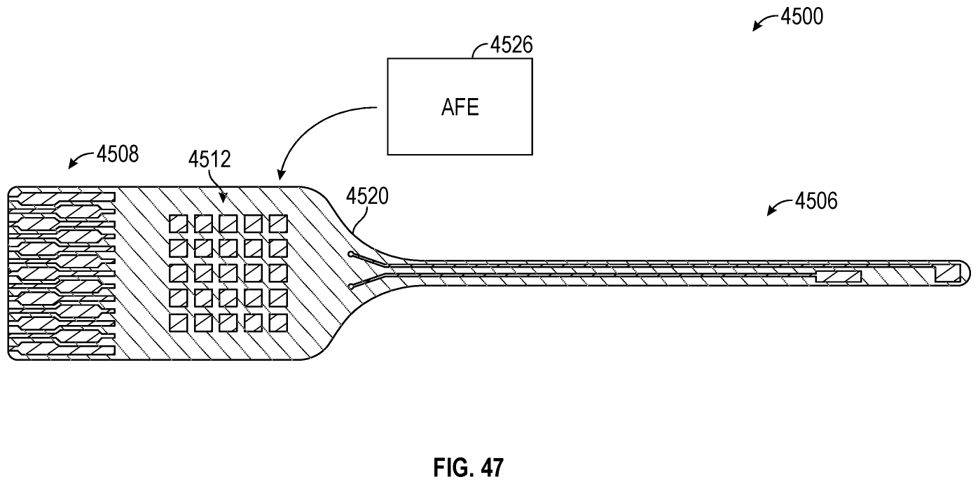

FIG. 47 is a diagram showing another example of the analyte sensor system 4500. In the example of FIG. 47, the analog front end circuit 4526 is shown removed from the sensor substrate 4520. The sensor substrate 4520 comprises a connector portion 4508 to interface with the connector 4130 to the sensor electronics 4106, as described herein. The sensor substrate 4520 also comprises pads 4512. The pads 4512 are arranged to interface with input/outputs of the analog front end circuit 4526. For example, the analog front end circuit 4526 may be electrically coupled to respective electrodes of the electrode subassembly 4506 via the pads 4512. Also, for example, some of the pads may be electrically coupled to traces running from respective electrodes. In some examples, the analog front end circuit 4526 may also be electrically coupled to the connector portion 4508 via the pads 4512. For example, the sensor substrate 4520 may comprise one or more traces connecting one or more of the pads 4512 to respective pads of the connector portion 4508.

In arrangements including an analog front end circuit coupled to a sensor substrate, as described herein, the sensor substrate, including the analog front end circuit, may be electrically coupled to the sensor electronics in any suitable manner. For example, a connector between the analyte sensor and the sensor electronics (e.g., the connector 4130), may be implemented with a connector portion that is electrically coupled to the analyte sensor and a connector portion that is electrically coupled to the sensor electronics. For example, FIGS. 44 and 47 show an arrangement in which a ZIF connector is used to electrically couple the analyte sensor (e.g. the sensor substrate) to the sensor electronics. Connector portions 4308 and 4508 of the analyte sensor may be configured to electrically and mechanically couple to a corresponding connector portion at the sensor electronics.

In arrangements such as the one described with respect to FIG. 2 in which analog front end circuitry is part of the sensor electronics 106 and arrangements similar to that shown in FIG. 41 in which the analog front end circuit 4126 is positioned on the analyte sensor 4104, passing a signal from the analyte sensor 104, 4104 to the sensor electronics 106, 4106 can be a challenge. For example, the connection between the analyte sensor 104, 4104 and the sensor electronics 106, 4106 may be sensitive to leakage, moisture, and other factors that affect noise. When the connection between the sensor electronics 106, 4106 and the analyte sensor 104, 4104 includes physical contacts, it may be desirable for such a connection (e.g., the connector 4130) to be hermetic. A contact-based hermetic connector may be bulky and costly to produce. This challenge may be addressed by placing the analog front end circuit 4126 at the analyte sensor for 104, as described herein, as this may result in a digital signal being passed across the connector 4130 instead of an analog signal. In some examples, however, the performance of the analyte sensor system may be further improved by utilizing a contactless connection. For example, the analyte sensor 4104 may be operatively coupled (e.g., electrically coupled) to the sensor electronics 4106 utilizing a contactless connection (e.g., wireless connection).

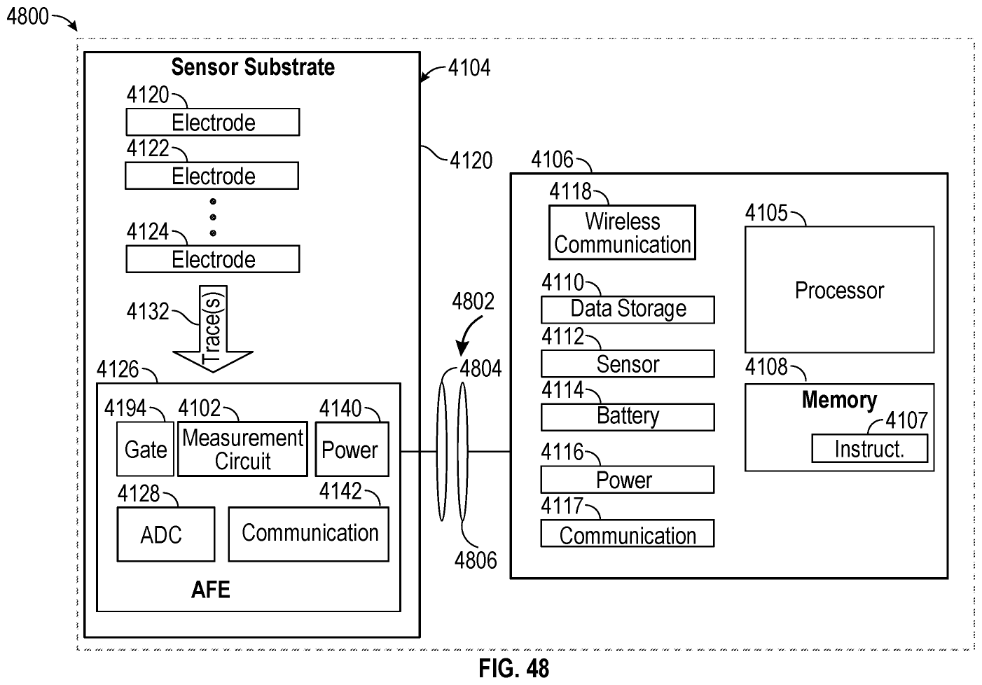

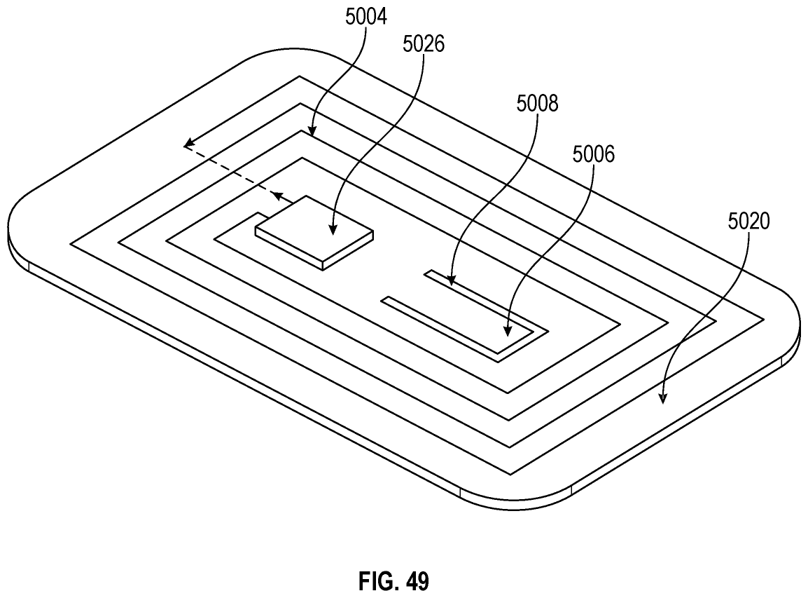

FIG. 48 is a diagram showing one example of an analyte sensor system 4800 including an analyte sensor 4104 electrically coupled to sensor electronics 4106 via a contactless connector 4802. In the example of FIG. 48, the analyte sensor 4104 and sensor electronics 4106 are arranged in a manner similar to that shown in FIG. 41. For example, as shown in FIGS. 41 and 48, the analog front end circuit 4126 is positioned on the sensor substrate 4120.

The contactless connector 4802 may be any suitable type of contactless connector. For example, the contactless connector 4802 may comprise a sensor-side element 4804 and an electronics-side element 4806. The sensor-side element 4804 may be electrically coupled to the analyte sensor 4104, such as, for example via the analog front end circuit 4126. The electronics-side element 4806 may be electrically coupled to the sensor electronics 4106. The sensor-side element 4804 and the electronics-side element 4806 may be positioned substantially close to each other to facilitate wireless communication between the sensor-side element 4804 and the electronics-side element 4806. In some examples, the sensor-side element 4804 and the electronics-side element 4806 may be in close proximity to each other, and may comprise one or more coils, antennas, or other similar elements that may be positioned in the analyte sensor system 4800 so as to cause the sensor-side element 4804 and the electronics-side element 4806 to be inductively coupled to one another without being in physical contact. It is contemplated that other antenna designs could be implemented to cause communications between the analyte sensor system 4800 and the sensor-side element 4804.

Positioning the sensor-side element 4804 and the electronics-side element 4806 substantially close to one another may include bringing the sensor-side element 4804 and the electronics-side element 4806 into proximity with one another and aligning the two elements 4804, 4806. For example, the sensor-side element 4804 and the electronics-side element 4806 may be positioned to generate a wireless connection when the elements 4804, 4806 are substantially overlapping one another and placed sufficiently close to one another that power and/or data signals may be transmitted wirelessly from one element 4804, 4806 and received by the other element 4804, 4806. Upon alignment of the sensor-side element 4804 and the transmitter-side element 4806, power and/or data may be transmitted across the contactless connector 4802. In some embodiments, communication circuit 4117 can send, receive, output, transmit, or the like data signals via electronics-side element 4806 to and from communication circuit 4142 via sensor-side element 4804 thereby enabling communication over contactless connector 4802. Power circuit 4116 can output power via electronics-side element 4806 to power circuit 4140 via sensor-side element 4804, thereby providing power to analyte sensor 4104 via contactless connector 4802.

The contactless connector 4802 may facilitate the transmission of data signals (for example, one way or bi-directional, e.g., between communication circuits 4117 and 4142) and power between the analog front end circuit 4126 and the sensor electronics 4106 (e.g., from power circuit 4116 to power circuit 4140). Data signals may include processed or unprocessed data. In one example, a digital output of the analog front end circuit 4126 such as, for example, as generated by the analog-to-digital converter 4128. Data signals may also include, for example, a clock signal, an interrupt signal, and/or the like. Power may be transmitted via the contactless connector 4802, for example, from the battery 4114. In some examples, the power requirements of the analyte sensor 4104 may be relatively small. In some cases, the distance between the sensor-side element 4804 and the electronics-side element 4806 of the contactless connector 4802 may be small, such as less than 10 mm, more preferably less than 5 mm, even more preferably less than 1 mm. Power may be provided constantly or in some cases on need basis. It is contemplated that, in some examples, the contactless connector 4802 may be configured to operate for short-range communication such as Radio Frequency ID (RFID) communication, or Near-Field Communication (NFC). In such examples, a suitable operating frequency (e.g., 13.56 MHZ, 433 MHz, 860-960 MHZ) may be selected for such operations. In the example of FIG. 48, the analog front end circuit 4126 is positioned at the analyte sensor 4104 and the signal across the contactless connector 4802 is digital. It will be appreciated that, in various examples, a contactless connector such as the contactless connector 4802 may be used in an arrangement in which the components of the analog front end circuit 4126 are positioned at the sensor electronics 4106 and the signal across the contactless connector 4802 is (in whole or in part) analog, similar to the arrangement shown in FIG. 2. For example, power may be provided to analyte sensor 4104 via NFC wireless power transfer.

Contactless connector 4802 advantageously allows for hermetically sealing of analyte sensor 4104 (e.g., and sensor substrate 4120) from the environment and from sensor electronics 4106. Contactless connector 4802 further advantageously allows for analyte sensor 4104 to be separate from a battery by allowing power to be transferred or provide from sensor electronic 4106. It is appreciated that the separation of a battery or other power source from analyte sensor 4104 can prevent or reduce the likelihood of an unintended or undesirable electrical path between a battery or other power source and one or more components of analyte sensor 4104. Sensor electronics 4106 thereby can be reused with one or more of analyte sensor 4104. In some embodiments, analyte sensor 4104 may be designed in a way such that it is relatively cheap cost wise and disposable.

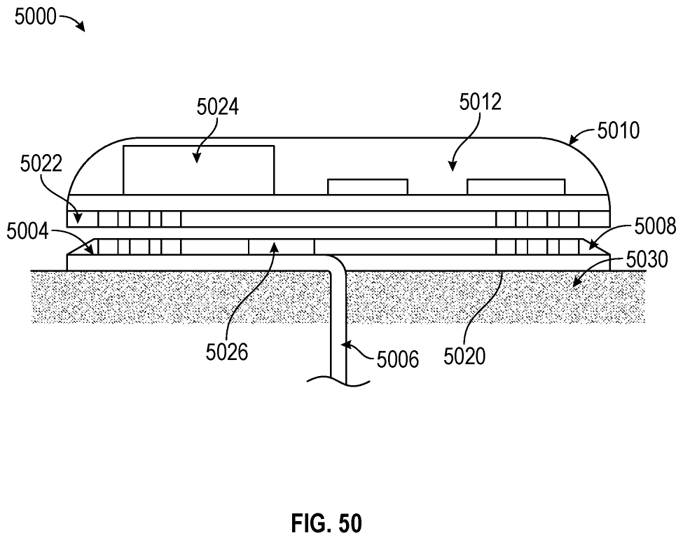

FIG. 49 is a diagram showing one arrangement of an analyte sensor system 5000 comprising a sensor substrate 5020 with a coil 5004 positioned thereon. The coil 5004, in some examples, comprises a trace positioned on the sensor substrate 5020 in the shape of a coil, or other suitable shape for inductively coupling to an electronics-side element. In the example of FIG. 50, the analog front end circuit 5026 is also positioned on the sensor substrate 5020 as described herein. In the arrangement of FIG. 49, the analyte sensor 5006 is positioned on the sensor substrate 5020. In this example, the analyte sensor 5006 is separated from a remainder of the sensor substrate 5020 via a cut 5008. The analyte sensor 5006 may be configured to bend low the remainder of the sensor substrate 5020 to facilitate insertion into a host as described herein.

FIG. 50 is a diagram showing another arrangement of the analyte sensor system 5000 including sensor electronics 5010. As shown, the sensor electronics 5010 include an electronics-side coil 5022 that is positioned to be inductively coupled to the sensor-side coil 5004. In the example of FIG. 50, sensor electronics 5010 comprise a battery 5024 and other components 5012, which may include, for example, any of the sensor electronics components described herein. The analog front end circuit 5026 is also shown. Further, FIG. 50 illustrates the analyte sensor 5006 bent distally relative to a remainder of the sensor substrate 5020 inserted into tissue 5030 of a host.

In various examples, the contactless connector arrangements shown in FIGS. 48-50 may remove the need for hermeticity between the sensor electronics and the analyte sensor, thereby improving noise performance reducing size and, potentially, cost. In some examples, a contactless connector arrangement, such as the arrangement shown in FIGS. 48-50 can be implemented with a reusable transmitter. In a reusable transmitter arrangement, all or part of the sensor electronics is positioned within an enclosure or housing, such as the electronics unit 318 shown in FIG. 3A. The transmitter enclosure or housing may be reused with multiple examples of the analyte sensor. For example, upon the expiration of a sensor session with one analyte sensor, the transmitter enclosure or housing (including the sensor electronics) may be removed from the analyte sensor and subsequently mechanically and electrically coupled to a new analyte sensor for a new sensor session. The old sensor, in some examples, is disposable.

In some examples, the analyte sensor for 104 and sensor electronics 4106 may be configured to begin operation upon the creation of the contactless connector 4802. For example, when the sensor-side element 4804 and electronics-side element 4806 are positioned to create the contactless connector 4802, the sensor electronics 4106 may begin transmitting power to the analyte sensor for 104. Similarly, upon creation of the contactless connector 4802, the analyte sensor 4104 may begin sending a sensor signal to the sensor electronics 4106. In some examples, the creation of the contactless connector 4802 occurs when a reusable transmitter enclosure is mechanically coupled to the analyte sensor for 104, thus positioning the sensor-side element 4804 and the electronics-side element 4806 to create the contactless connector 4802.

The use of a contactless connector, as described with respect to FIGS. 48-50, in an arrangement with a reusable transmitter and a disposable analyte sensor can, in some examples, lead to simpler and less expensive designs for both parts. For example, instead of separate electrical and mechanical coupling mechanisms, such an arrangement may use a mechanical coupling arrangement that also aligns the sensor-side element 4804 and the electronics-side element 4806.

In some examples, the sensor electronics may be fabricated on a sensor electronic substrate. The sensor electronics substrate may comprise a side load connector portion. The side load connector portion may receive an analyte sensor connector portion, such as for example, one of the connector portions 4308, 4508 described herein.

In some examples, the connector portion at the analyte sensor may comprise a plurality of pins and the connector portion at the sensor electronics may comprise a plurality of receptacles. The analyte sensor may be positioned such that the pins are mechanically received within the receptacles to generate an electrical and mechanical coupling of the analyte sensor and the sensor electronics. In some arrangements, pins may comprise a sharp end. The receptacles may comprise a conductive elastomer. The connector portion from the analyte sensor may be coupled to the connector portion from the sensor electronics by pressing the sharp end of the pins into the conductive elastomer.

In some examples, the connector portion of the analyte sensor and/or the connector portion of the sensor electronics comprises a flexible cable. The flexible cable may be made from wire and/or may be printed onto a flexible substrate. When the two connector portions are joined, the flexible cable may facilitate positioning of the analyte sensor relative to the sensor electronics.

In some examples, the connector portion of the analyte sensor may comprise one or more conductive tabs. The connector portion of the sensor electronics may have corresponding conductive tabs. The respective conductive tabs may be arranged to be mechanically resilient. The analyte sensor and/or the sensor electronics may additionally comprise a snap mechanism. When the conductive tabs of the analyte sensor are brought into contact with the conductive tabs of the sensor electronics, the respective tabs may provide resistance until the snap mechanism engages to hold the analyte sensor and the sensor electronics in a mechanically coupled position. Electrical coupling between the analyte sensor and the sensor electronics may be provided by the contact between the conductive tabs.

In some examples, the connector portion of the analyte sensor and/or the connector portion of the sensor electronics comprises a gasket or other similar sealing mechanism. When the two connector portions are brought into contact, the gasket or other similar sealing mechanism may engage to protect the connections from moisture and/or other environmental factors.

In some examples, the connector portion of the analyte sensor and/or the connector portion sensor electronics may comprise a conductive adhesive film. When the two connector portions are brought into mechanical contact with one another, the conductive adhesive films may mechanically and electrically couple the connector portions.

### Various Notes & Examples

Example 1 is a continuous analyte sensor comprising: a substrate having a first side and a second side opposite the first side, wherein the substrate is planar; a first working electrode on the substrate; a second working electrode on the substrate; a reference electrode on the substrate, wherein the first working electrode, the second working electrode, and the reference electrode are each planar electrodes, wherein at least two of the first working electrode, the second working electrode, or the reference electrode are on the first side of the substrate, and any remaining planar electrodes are on the second side of the substrate, wherein the at least two of first working electrode, the second working electrode, and the reference electrode are co-planar with each other; and an interconnect extending through the substrate between the first side and the second side, the interconnect in electrical communication with one of the reference electrode, the first working electrode, or the second working electrode.

In Example 2, the subject matter of Example 1 optionally includes wherein the first working electrode and the second working electrode are on the first side of the substrate and the reference electrode is on the second side of the substrate.

In Example 3, the subject matter of Example 2 optionally includes wherein the first working electrode and the second working electrode are co-planar with each other.

In Example 4, the subject matter of any one or more of Examples 1-3 optionally include wherein the first working electrode and the reference are on the first side of the substrate and the second reference electrode is on the second side of the substrate.

In Example 5, the subject matter of Example 4 optionally includes wherein the first working electrode and the reference electrode are co-planar with each other.

In Example 6, the subject matter of any one or more of Examples 1-5 optionally include wherein the first working electrode spaced distally from the second working electrode on the substrate.

In Example 7, the subject matter of any one or more of Examples 1-6 optionally include wherein the first working electrode and the second working electrode each have an approximately equal surface area.

In Example 8, the subject matter of any one or more of Examples 1-7 optionally include wherein the reference electrode has a smaller surface area relative to a surface area of either of the first working electrode and the second working electrode.

In Example 9, the subject matter of any one or more of Examples 1-8 optionally include a second reference electrode.

In Example 10, the subject matter of Example 9 optionally includes wherein the first working electrode and the second working electrode are on the first side of the substrate, and the first reference electrode and the second reference electrode are on the second side of the substrate.

In Example 11, the subject matter of any one or more of Examples 1-10 optionally include wherein the first reference electrode has a smaller surface area relative to a surface area of the second reference electrode.

In Example 12, the subject matter of any one or more of Examples 1-11 optionally include a third working electrode, wherein the third working electrode is co-planar with at least one of the first working electrode, the second working electrode, or the reference electrode.

In Example 13, the subject matter of Example 12 optionally includes wherein the first working electrode, the second working electrode, and the third working electrode are on the first side of the substrate.

In Example 14, the subject matter of Example 13 optionally includes wherein the first working electrode is spaced apart from the second working electrode by a first distance on the substrate, and the second working electrode is spaced apart from the third working electrode by a second distance on the substrate.

In Example 15, the subject matter of Example 14 optionally includes wherein the first distance and the second distance are approximately equal.

In Example 16, the subject matter of any one or more of Examples 12-15 optionally include wherein the first working electrode and the second working electrode are on the first side of the substrate, and the third working electrode is on the second side of the substrate.

In Example 17, the subject matter of any one or more of Examples 12-16 optionally include wherein the first working electrode, the second working electrode, and the third working electrode each have an approximately equal surface area.

In Example 18, the subject matter of any one or more of Examples 1-17 optionally include a first counter electrode, wherein the first counter electrode is a planar electrode.

In Example 19, the subject matter of Example 18 optionally includes wherein the first counter electrode is co-planar with at least one of the first working electrode, the second working electrode, or the reference electrode.

In Example 20, the subject matter of any one or more of Examples 18-19 optionally include wherein the first working electrode and the second working electrode are on the first side of the substrate, and wherein the reference electrode and the counter electrode are on the second side of the substrate.

In Example 21, the subject matter of Example 20 optionally includes wherein the counter electrode is spaced distally from the reference electrode on the substrate.

In Example 22, the subject matter of any one or more of Examples 18-21 optionally include wherein the counter electrode has a larger surface area than the reference electrode.

In Example 23, the subject matter of any one or more of Examples 18-22 optionally include a second counter electrode, the first counter electrode and the second counter electrode each being planar electrodes.

In Example 24, the subject matter of any one or more of Examples 1-23 optionally include wherein the first working electrode, the second working electrode, and the reference electrode are aligned longitudinally along the substrate.

In Example 25, the subject matter of any one or more of Examples 1-24 optionally include at least one analyte-sensing membrane extending over one or more of the first working electrode and the second working electrode.

In Example 26, the subject matter of Example 25 optionally includes at least one analyte-sensing membrane comprises an enzymatic layer configured to sense glucose.

In Example 27, the subject matter of any one or more of Examples 25-26 optionally include wherein the at least one analyte-sensing membrane comprises an enzymatic layer configured to sense lactate.

In Example 28, the subject matter of any one or more of Examples 25-27 optionally include wherein the at least one analyte-sensing membrane comprises is configured to sense more than one analyte.

In Example 29, the subject matter of any one or more of Examples 1-28 optionally include wherein each of the first working electrode, the second working electrode, and the reference electrode has a border on the substrate.

In Example 30, the subject matter of any one or more of Examples 1-29 optionally include wherein the substrate comprises a tip portion extending distally from the first working electrode, the second working electrode, and the reference electrode.

In Example 31, the subject matter of Example 30 optionally includes wherein the substrate further comprises a tip portion of the substrate not including the first working electrode, the second working electrode, and the reference electrode.

In Example 32, the subject matter of any one or more of Examples 30-31 optionally include wherein the tip portion is perforated.

In Example 33, the subject matter of any one or more of Examples 30-32 optionally include wherein the tip portion is rounded.

In Example 34, the subject matter of any one or more of Examples 30-33 optionally include wherein the tip portion comprises a soft material.

In Example 35, the subject matter of any one or more of Examples 30-34 optionally include wherein the tip portion comprises a cap secured to the substrate at a distal end.

In Example 36, the subject matter of any one or more of Examples 30-35 optionally include wherein the tip portion is bendable.

In Example 37, the subject matter of Example 36 optionally includes wherein the tip portion comprises an indent and a rounded portion, the rounded portion spaced distally from the indent.

Example 38 is a sensor comprising: a substrate having a first side and a second side opposite the first side, wherein the substrate is planar; a first sensor system on the substrate, the first sensor system being a continuous analyte sensor configured to collect a first type of measurement, wherein the first sensor system comprises: a working electrode; a reference electrode on the substrate, wherein the working electrode and the reference electrode are each planar electrodes; and at least one analyte-sensing membrane extending over the at least one working electrode; and a second sensor system on the substrate, wherein the second sensor system is configured to collect a second type of measurement different than the first type of measurement.

In Example 39, the subject matter of Example 38 optionally includes wherein the first sensor system comprises an amperometric sensor.

In Example 40, the subject matter of Example 39 optionally includes wherein the second sensor system comprises a potentiometric sensor.

In Example 41, the subject matter of Example 40 optionally includes wherein the second sensor system comprises: a first electrode disposed on the substrate; an ionophore disposed on the substrate and configured to selectively transport a target ion to or within the first electrode; and a second electrode disposed on the substrate.

In Example 42, the subject matter of Example 41 optionally includes sensor electronics configured to: generate a first signal corresponding to a current response, the current response being at least partially based on a reaction at the working electrode corresponding to a concentration of a first analyte, and generate a second signal corresponding to an electromotive force, the electromotive force being at least partially based on a potential difference that is generated between the first electrode and the second electrode responsive to the ionophore transporting the target ion corresponding to a concentration of a second analyte to the first electrode.

In Example 43, the subject matter of any one or more of Examples 41-42 optionally include wherein the first electrode of the second sensor system is on the first side of the substrate, and the second electrode of the second sensor system is on the second side of the substrate.

In Example 44, the subject matter of any one or more of Examples 41-43 optionally include wherein the first sensor system is on the first side of the substrate, and the second sensor system is on the second side of the substrate.

In Example 45, the subject matter of any one or more of Examples 41-44 optionally include wherein the first sensor system and the second sensor system are both on the first side of the substrate.

In Example 46, the subject matter of any one or more of Examples 38-45 optionally include a third sensor system.

In Example 47, the subject matter of Example 46 optionally includes wherein the third sensor system is configured to collect a third type of measurement different than the first and second types of measurements.

In Example 48, the subject matter of any one or more of Examples 46-47 optionally include wherein the first sensor system and the third sensor system are amperometric sensors.

In Example 49, the subject matter of any one or more of Examples 46-48 optionally include wherein the second sensor system and the third sensor system are potentiometric sensors.

In Example 50, the subject matter of any one or more of Examples 38-49 optionally include wherein the first sensor system comprises at least two working electrodes.

In Example 51, the subject matter of any one or more of Examples 38-50 optionally include wherein the first sensor system comprises at least three working electrodes.

In Example 52, the subject matter of any one or more of Examples 38-51 optionally include wherein the first sensor system further comprises a counter electrode.

In Example 53, the subject matter of any one or more of Examples 38-52 optionally include wherein the first sensor system further comprises two counter electrodes.

In Example 54, the subject matter of any one or more of Examples 38-53 optionally include wherein the first sensor system further comprises a second reference electrode.

In Example 55, the subject matter of any one or more of Examples 38-54 optionally include at least one interconnect extending between the first side of the substrate and the second side of the substrate, the at least one interconnect in electrical communication with one of the reference electrode or the working electrode.

In Example 56, the subject matter of any one or more of Examples 38-55 optionally include wherein the working electrode and the reference electrode are co-planar with each other.

In Example 57, the subject matter of any one or more of Examples 38-56 optionally include wherein the working electrode is on the first side of the substrate and the reference electrode is on the second side of the substrate.

In Example 58, the subject matter of any one or more of Examples 38-57 optionally include wherein the working electrode and the reference electrode are on the first side of the substrate.

Example 59 is a continuous analyte sensor comprising: a planar substrate having a distal portion and a proximal portion connected by a junction portion, wherein the distal portion and the proximal portion are connected through the junction portion at an angle between 70 and 110 degrees; a first electrode on the distal portion of the substrate and a first connection pad on the proximal portion, the first electrode and the first connection pad electrically coupled through a first trace routed through the junction portion; a second electrode on the distal portion of the substrate and a second connection pad on the proximal portion, the second electrode and the second connection pad electrically coupled through a second trace routed through the junction portion; and at least one analyte-sensing membrane extending over one or more of the first electrode and the second electrode.

In Example 60, the subject matter of Example 59 optionally includes wherein the proximal portion has a width larger than a width of the distal portion.

In Example 61, the subject matter of any one or more of Examples 59-60 optionally include wherein the proximal portion and the distal portion are of approximately the same length.

In Example 62, the subject matter of any one or more of Examples 59-61 optionally include wherein the proximal portion comprises a folded portion having a first planar portion and a second planar portion folded on top of the first planar portion.

In Example 63, the subject matter of Example 62 optionally includes wherein the first connection pad and the second pad are on the folded portion.

In Example 64, the subject matter of any one or more of Examples 62-63 optionally include wherein the folded portion comprises an interconnect extending between each side of the folded portion, the interconnect in electrical communication with one of the first connection pad or second connection pad.

In Example 65, the subject matter of any one or more of Examples 59-64 optionally include wherein the proximal portion further comprises a stiffener.

In Example 66, the subject matter of any one or more of Examples 59-65 optionally include one or more additional electrical elements on the distal portion.

In Example 67, the subject matter of any one or more of Examples 59-66 optionally include wherein the first trace and the second trace are routed through the junction portion.

In Example 68, the subject matter of any one or more of Examples 59-67 optionally include wherein the first electrode and the second electrode are planar electrodes.

In Example 69, the subject matter of any one or more of Examples 59-68 optionally include wherein one of the first electrode or the second electrode is a working electrode.

In Example 70, the subject matter of any one or more of Examples 59-69 optionally include wherein one of the first electrode or the second electrode is a reference electrode.

In Example 71, the subject matter of any one or more of Examples 59-70 optionally include a third electrode on the distal portion.

In Example 72, the subject matter of Example 71 optionally includes wherein the third electrode comprises a working electrode, a reference electrode, or a counter electrode.

In Example 73, the subject matter of any one or more of Examples 59-72 optionally include wherein the first electrode is on a first side of the distal portion, and the second electrode is on a second side of the distal portion opposite the first side.

In Example 74, the subject matter of any one or more of Examples 59-73 optionally include wherein the first electrode and the second electrode are co-planar with each other.

In Example 75, the subject matter of any one or more of Examples 59-74 optionally include wherein the at least one analyte-sensing membrane comprises an enzymatic layer configured to sense glucose.

In Example 76, the subject matter of any one or more of Examples 59-75 optionally include wherein the at least one analyte-sensing membrane comprises an enzymatic layer configured to sense lactate.

In Example 77, the subject matter of any one or more of Examples 59-76 optionally include a third electrode, wherein the first electrode and the third electrode are working electrodes, and wherein the at least one analyte-sensing membrane comprises one or more enzymatic layers configured to sense more than one analyte.

In Example 78, the subject matter of any one or more of Examples 59-77 optionally include wherein the planar substrate comprises a tip portion distal of the first electrode and the second electrode.

Example 79 is a method of making a planar analyte sensor, the method comprising: layering a first insulating material, a first conductive material, and a second conductive material on a first side of a substrate; exposing a portion of first conductive material to form a first electrode by selectively removing portions of the first insulating material; exposing a portion of the second conductive material to form a second electrode by selectively removing portions of the first insulating material; and layering a second insulating material and a third conductive material on a second side of the substrate opposite the first side; exposing a portion of the third conductive material to form a third electrode by selectively removing portions of the third insulating material; and depositing an analyte-sensing membrane onto two of the first electrode, the second electrode, or the third electrode.

In Example 80, the subject matter of Example 79 optionally includes wherein depositing the analyte-sensing membrane comprises jet valve dispensing the analyte-sensing membrane.

In Example 81, the subject matter of any one or more of Examples 79-80 optionally include wherein depositing the analyte-sensing membrane comprises slot die coating.

In Example 82, the subject matter of any one or more of Examples 79-81 optionally include wherein depositing the analyte-sensing membrane comprises screen printing.

In Example 83, the subject matter of any one or more of Examples 79-82 optionally include wherein depositing an analyte-sensing membrane comprises depositing a multi-layer membrane with an enzyme layer containing one or more analyte-sensitive enzymes.

In Example 84, the subject matter of Example 83 optionally includes an additional enzyme layer containing one or more analyte-sensitive enzymes.

In Example 85, the subject matter of any one or more of Examples 83-84 optionally include wherein the enzyme layer is configured to detect glucose.

In Example 86, the subject matter of any one or more of Examples 83-85 optionally include wherein the intermediate layer is configured to detect lactate.

In Example 87, the subject matter of any one or more of Examples 79-86 optionally include making an interconnect through the substrate from the first side to the second side, the interconnect in electrical communication with one of the first electrode, the second electrode, or the third electrode.

Example 88 is a method of making a plurality of analyte sensors, the method comprising: producing a plurality of sensor substrates from a substrate material sheet, wherein each of the plurality of sensor substrates is aligned on the substrate material sheet; forming a working electrode and a reference electrode on each of the plurality of sensor substrates; applying an analyte-sensitive membrane on each of the working electrodes on each of the plurality of sensor substrates.

In Example 89, the subject matter of Example 88 optionally includes wherein applying the analyte-sensitive membrane comprises jet valve dispensing.

In Example 90, the subject matter of any one or more of Examples 88-89 optionally include wherein applying the analyte-sensitive membrane comprises slot die coating.

In Example 91, the subject matter of any one or more of Examples 88-90 optionally include wherein applying the analyte-sensitive membrane comprises screen printing.

In Example 92, the subject matter of any one or more of Examples 88-91 optionally include singulating analyte sensors from substrate material sheet.

Example 93 is a method of making an analyte sensor, the method comprising: aligning a plurality of insulating layers and a plurality of conducting layers in an alternating fashion; laminating the plurality of insulating layers and the plurality of conductive layers together; and exposing at least two electrodes by selectively removing portions of the plurality of insulating layers.

In Example 94, the subject matter of Example 93 optionally includes electroplating the at least two electrodes.

In Example 95, the subject matter of any one or more of Examples 93-94 optionally include rotogravure one of the at least two electrodes to form a reference electrode.

In Example 96, the subject matter of any one or more of Examples 93-95 optionally include wherein exposing at least two electrodes comprises laser skiving.

In Example 97, the subject matter of any one or more of Examples 93-96 optionally include singulating the analyte sensor from the plurality of insulating layers and the plurality of conductive layers.

In Example 98, the subject matter of any one or more of Examples 93-97 optionally include wherein the method is a roll-to-roll method.

Example 99 is an analyte sensor comprising: a sensor substrate; a first electrode mechanically coupled to the sensor substrate; a first electrode trace mechanically coupled to the sensor substrate and electrically coupled to the first electrode; a second electrode mechanically coupled to the sensor substrate; a second electrode trace mechanically coupled to the sensor substrate and electrically coupled to the first electrode; and an analog front end (AFE) circuit mechanically coupled to the sensor substrate, electrically coupled to the first electrode trace, and electrically coupled to the second electrode trace.

In Example 100, the subject matter of Example 99 optionally includes the AFE circuit comprising an AFE substrate, the AFE substrate being mechanically coupled to the sensor substrate.

In Example 101, the subject matter of any one or more of Examples 99-100 optionally include the AFE circuit comprising an analog-to-digital converter electrically coupled to convert an analog electrical signal generated by the first electrode and the second electrode to a digital signal.

In Example 102, the subject matter of Example 101 optionally includes an output connector to couple the analyte sensor to a sensor electronics assembly, the AFE circuit comprising: a first analog input electrically coupled to the first electrode trace; a second analog input electrically coupled to the second electrode trace; and at least one digital output electrically coupled to output connector.

In Example 103, the subject matter of Example 102 optionally includes the first analog input and the second analog input being positioned on a first side of the AFE circuit, the first side of the AFE circuit being bonded to the sensor substrate.

In Example 104, the subject matter of any one or more of Examples 99-103 optionally include the AFE circuit comprising a power input, the analyte sensor further comprising a first power conditioning capacitor coupled to the sensor substrate and electrically connected to the power input.

In Example 105, the subject matter of any one or more of Examples 99-104 optionally include an enclosure mechanically coupled to the sensor substrate, the AFE circuit being positioned within the enclosure.

In Example 106, the subject matter of Example 105 optionally includes the enclosure being bonded to the sensor substrate using a bonding agent.

In Example 107, the subject matter of any one or more of Examples 105-106 optionally include the enclosure being molded on the sensor substrate.

Example 108 is an analyte sensor system comprising: an analyte sensor, the analyte sensor comprising: a sensor substrate; a first electrode mechanically coupled to the sensor substrate; a first electrode trace mechanically coupled to the sensor substrate and electrically coupled to the first electrode; a second electrode mechanically coupled to the sensor substrate; a second electrode trace mechanically coupled to the sensor substrate and electrically coupled to the first electrode; and an analog front end (AFE) circuit mechanically coupled to the sensor substrate, electrically coupled to the first electrode trace, and electrically coupled to the second electrode trace; and a sensor electronics assembly; and a connector electrically coupling the analog front end circuit to the sensor electronics assembly.

In Example 109, the subject matter of Example 108 optionally includes the connector being a zero input force (ZIF) connector.

In Example 110, the subject matter of any one or more of Examples 108-109 optionally include the sensor electronics assembly receive at least one digital signal from the AFE across the connector.

In Example 111, the subject matter of any one or more of Examples 108-110 optionally include the AFE circuit comprising an AFE substrate, the AFE substrate being mechanically coupled to the sensor substrate.

In Example 112, the subject matter of any one or more of Examples 108-111 optionally include the AFE circuit comprising an analog-to-digital converter electrically coupled to convert an analog electrical signal generated by the first electrode and the second electrode to a digital signal.

In Example 113, the subject matter of Example 112 optionally includes an output connector to couple the analyte sensor to a sensor electronics assembly, the AFE circuit comprising: a first analog input electrically coupled to the first electrode trace; a second analog input electrically coupled to the second electrode trace; and at least one digital output electrically coupled to output connector.

In Example 114, the subject matter of Example 113 optionally includes the first analog input and the second analog input being positioned on a first side of the AFE circuit, the first side of the AFE circuit being bonded to the sensor substrate.

In Example 115, the subject matter of any one or more of Examples 108-114 optionally include the AFE circuit comprising a power input, the analyte sensor further comprising a first power conditioning capacitor coupled to the sensor substrate and electrically connected to the power input.

In Example 116, the subject matter of any one or more of Examples 108-115 optionally include an enclosure mechanically coupled to the sensor substrate, the AFE circuit being positioned within the enclosure.

In Example 117, the subject matter of Example 116 optionally includes the enclosure being bonded to the sensor substrate using a bonding agent.

In Example 118, the subject matter of any one or more of Examples 116-117 optionally include the enclosure being molded on the sensor substrate.

In Example 119, the subject matter of any one or more of Examples 108-118 optionally include the connector comprising a sensor-side contact and an electronics-side contact, the sensor-side contact and the electronics-side contact being in physical contact with one another to electrically couple the analog front end circuit to the sensor electronics assembly.

In Example 120, the subject matter of any one or more of Examples 108-119 optionally include the connector being a contactless connector.

In Example 121, the subject matter of Example 120 optionally includes the connector comprising a sensor-side element and an electronics-side element, the sensor-side element and the electronics-side element being positioned to inductively couple the sensor-side element and the electronics-side element.

In Example 122, the subject matter of any one or more of Examples 120-121 optionally include the analyte sensor being configured to receive power from the sensor electronics via the contactless connector.

Example 123 is a method of providing data from an analyte sensor to sensor electronics, comprising: positioning a sensor-side element of a contactless connector and an electronics-side element of the contactless connector to generate a wireless connection between the sensor-side element and the electronics-side element; transmitting power from the sensor electronics to the analyte sensor via the wireless connection; and transmitting a data signal from the analyte sensor to the sensor electronics via the wireless connection.

In Example 124, the subject matter of Example 123 optionally includes the sensor electronics being positioned in an electronics unit enclosure, the method further comprising: mechanically coupling the electronics unit enclosure to the analyte sensor, the transmitting of power from the sensor electronics to the analyte sensor being responsive to the mechanical coupling.

In Example 125, the subject matter of any one or more of Examples 123-124 optionally include the sensor electronics being positioned in an electronics unit enclosure, the method further comprising: mechanically coupling the electronics unit enclosure to the analyte sensor, the transmitting of the data signal from the sensor electronics to the analyte sensor being responsive to the mechanical coupling.

In Example 126, the subject matter of any one or more of Examples 123-125 optionally include the data signal being a digital signal.

In Example 127, the subject matter of any one or more of Examples 123-126 optionally include the data signal being an analog signal.

The above detailed description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present subject matter also contemplates examples in which only those elements shown or described are provided. Moreover, the present subject matter also contemplates examples using any combination or permutation of those elements shown or described (or one or more aspects thereof), cither with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An analyte sensor system comprising:
an analyte sensor, the analyte sensor comprising:
a sensor substrate;
a first electrode mechanically coupled to the sensor substrate;
a first electrode trace mechanically coupled to the sensor substrate and electrically coupled to the first electrode;
a second electrode mechanically coupled to the sensor substrate;
a second electrode trace mechanically coupled to the sensor substrate and electrically coupled to the first electrode; and
an analog front end (AFE) circuit mechanically coupled to the sensor substrate, electrically coupled to the first electrode trace, and electrically coupled to the second electrode trace; and
a sensor electronics assembly; and
a connector electrically coupling the analog front end circuit to the sensor electronics assembly.

2. The analyte sensor system of claim 1, wherein the connector is a zero input force (ZIF) connector.

3. The analyte sensor system of claim 1, wherein the sensor electronics assembly receives at least one digital signal from the AFE across the connector.

4. The analyte sensor system of claim 1, wherein the AFE circuit comprises an AFE substrate that is mechanically coupled to the sensor substrate.

5. The analyte sensor system of claim 1, wherein the AFE circuit comprises an analog-to-digital converter electrically coupled to convert an analog electrical signal generated by the first electrode and the second electrode to a digital signal.

6. The analyte sensor system of claim 5, further comprising an output connector to couple the analyte sensor to a sensor electronics assembly, wherein the AFE circuit comprising:
a first analog input electrically coupled to the first electrode trace;
a second analog input electrically coupled to the second electrode trace; and
at least one digital output electrically coupled to output connector.

7. The analyte sensor system of claim 6, wherein:
the first analog input and the second analog input are positioned on a first side of the AFE circuit, and
the first side of the AFE circuit is bonded to the sensor substrate.

8. The analyte sensor system of claim 1, wherein:
the AFE circuit comprises a power input, and
the analyte sensor further comprises a first power conditioning capacitor coupled to the sensor substrate and electrically connected to the power input.

9. The analyte sensor system of claim 1, further comprising an enclosure mechanically coupled to the sensor substrate, wherein the AFE circuit is positioned within the enclosure.

10. The analyte sensor system of claim 9, wherein the enclosure is bonded to the sensor substrate using a bonding agent.

11. The analyte sensor system of claim 9, wherein the enclosure is molded on the sensor substrate.

12. The analyte sensor system of claim 1, wherein:
the connector comprises a sensor-side contact and an electronics-side contact, and
the sensor-side contact and the electronics-side contact are in physical contact with one another to electrically couple the analog front end circuit to the sensor electronics assembly.

13. The analyte sensor system of claim 1, the connector being a contactless connector.

14. The analyte sensor system of claim 13, wherein:
the connector comprises a sensor-side element and an electronics-side element, and
the sensor-side element and the electronics-side element are positioned to inductively couple the sensor-side element and the electronics-side element.

15. The analyte sensor system of claim 13, wherein the analyte sensor is configured to receive power from the sensor electronics via the contactless connector.

16. The analyte sensor system of claim 1, wherein the AFE circuit comprises an AFE power circuit, coupled to the connector, for receiving power from the sensor electronics assembly.

17. The analyte sensor system of claim 1, wherein the AFE circuit comprises an AFE communication circuit, coupled to the connector, for communicating with the sensor electronics assembly.

18. The analyte sensor system of claim 1, wherein the sensor electronics assembly comprises a sensor electronics power circuit, coupled to the connector, for providing power to the AFE circuit.

19. The analyte sensor system of claim 1, wherein the sensor electronics assembly comprises a sensor electronics communication circuit, coupled to the connector, for communicating with the AFE.

20. A method of providing data from an analyte sensor to sensor electronics, comprising:
positioning a sensor-side element of a contactless connector and an electronics-side element of the contactless connector to generate a wireless connection between the sensor-side element and the electronics-side element;
transmitting power from the sensor electronics to the analyte sensor via the wireless connection; and
transmitting a data signal from the analyte sensor to the sensor electronics via the wireless connection.

21. The method of claim 20, wherein:
the sensor electronics is positioned in an electronics unit enclosure,
the method further comprises mechanically coupling the electronics unit enclosure to the analyte sensor, and
transmitting power from the sensor electronics to the analyte sensor is responsive to the mechanical coupling.

22. The method of claim 20, wherein:
the sensor electronics is positioned in an electronics unit enclosure,
the method further comprises mechanically coupling the electronics unit enclosure to the analyte sensor, and
transmitting the data signal from the sensor electronics to the analyte sensor is responsive to the mechanical coupling.

23. The method of claim 20, wherein the data signal is a digital signal.

24. The method of claim 20, wherein the data signal is an analog signal.
